SEQUENCE LISTING (6 sequences)

SEQ ID NO: 1 - protein, 20 aa, Artificial Sequence
  Synthetic construct
VTIFVAGVLTASLTIWKKMG

SEQ ID NO: 2 - protein, 10 aa, Artificial Sequence
  Synthetic construct
ASLTIWKKMG

SEQ ID NO: 3 - protein, 10 aa, Artificial Sequence
  Synthetic construct
VTIFVAGVLT

SEQ ID NO: 4 - protein, 7 aa, Artificial Sequence
  Synthetic construct
VTIFVAG

SEQ ID NO: 5 - protein, 5 aa, Artificial Sequence
  Synthetic construct
IFVAG

SEQ ID NO: 6 - protein, 6 aa, Artificial Sequence
  Synthetic construct
IWKKMG

(19) United States
(12) Patent Application Publication
(10) Pub. No.: US 2024/0122867 A1
(43) Pub. Date: Apr. 18, 2024

(54) CORONAL PROTEIN-COATED NANOPARTICLES AND USES THEREOF

(71) Applicant: UNIVERSITY OF CENTRAL FLORIDA RESEARCH FOUNDATION, INC., Orlando, FL (US)

(72) Inventors: Annette KHALED, Orlando, FL (US); Lei ZHAI, Orlando, FL (US); Kai MCKINSTRY, Orlando, FL (US); Daniel NIERENBERG, Orlando, FL (US)

(21) Appl. No.: 18/276,929

(22) PCT Filed: Feb. 14, 2022
(86) PCT No.: PCT/US2022/016301

§ 371 Date: Aug. 11, 2023

Related U.S. Application Data
(60) Provisional application No. 63/149,094, filed on Feb. 12, 2021.
(60) Provisional application No. 63/284,786, filed on Dec. 1, 2021.

(51) Int. Cl. A61K 9/51; A61K 31/337; A61P 35/00

(52) U.S. Cl. CPC A61K 9/5169; A61K 31/337; A61P 35/00

(57) ABSTRACT

Disclosed are nanoparticle compositions and methods for treating cancer. Further, hyperbranched polyester (HBPE) nanoparticle that comprise one or more coronal proteins are disclosed herein.

### CROSS REFERENCE TO RELATED APPLICATIONS

This application claims the benefit of priority to U.S. Provisional Application No. 63/149,094, filed Feb. 12, 2021, and U.S. Provisional Application No. 63/284,786, filed Dec. 1, 2021, which are both incorporated by reference herein in their entireties.

### FIELD

The subject matter disclosed herein is in the field of nanoparticles, including compositions comprising coronal protein-coated nanoparticles and uses thereof.

### BACKGROUND

Nanoparticles (NPs) are nanometer (nm)-sized molecules that can comprise a variety of materials, sizes and shapes. NPs have been used for environmental, manufacturing, electronics, optics, and medical applications, including, for example, cancer therapy. However, it is estimated that less than 1% of NPs that are initially injected in animal models are capable of reaching tumors. As a result, high delivery efficiency is required for drugs to be effective. Thus, there is a need for compositions and methods for the delivery of therapeutic agents to tumors with nanoparticles. These needs and other needs are satisfied by the present invention.

### SUMMARY

In accordance with the purposes of the disclosed materials, compounds, compositions, articles, devices, and methods, as embodied and broadly described herein, the disclosed subject matter relates to compositions and methods of making and using the compositions.

Disclosed herein is a coronal protein-coated nanoparticle comprising one or more proteins, wherein the nanoparticle is a hyperbranched polyester (HBPE) nanoparticle (NP). In some examples, the one or more proteins are selected from the group consisting of complement C3, alpha-2-HS-glycoprotein, complement factor B, vitronectin, clusterin, inhibitor of carbonic anhydrase, H-2 class I histocompatibility antigen, Q10 alpha chain, complement C5, carboxypeptidase N subunit 2, plasma protease C1 inhibitor, alpha-1-acid glycoprotein 1, alpha-2-antiplasmin, complement component C8 alpha chain, complement component C9, serum amyloid A-1 protein, complement factor D, serum amyloid A-2 protein, Ig-like domain-containing protein, complement C1s-A subcomponent, N-acetylmuramoyl-L-alanine amidase, carboxypeptidase N catalytic chain, complement C2, complement component 7, mannan-binding lectin serine protease 2, ficolin-1, complement C1r-A subcomponent, vitamin K-dependent protein S, mannan-binding lectin serine protease 1, glyceraldehyde-3-phosphate dehydrogenase, vitamin K-dependent protein C, interleukin-1 receptor accessory protein, fibronectin, apolipoprotein B-100, complement factor H, haptoglobin, immunoglobulin heavy constant mu, complement component C8 beta chain, Ig gamma-2B chain C region, protein AMBP, Ig gamma-1 chain C region, membrane-bound form, complement component C8 gamma chain, alpha-1-acid glycoprotein 2, immunoglobulin kappa constant, mannose-binding protein C, beta-2-microglobulin, serum amyloid P-component, complement C1s-B subcomponent, transthyretin, inter alpha-trypsin inhibitor, heavy chain 4, Inter-alpha-trypsin inhibitor heavy chain H2, histidine-rich glycoprotein, afamin, apolipoprotein A-II, corticosteroid-binding globulin, flavin reductase (NADPH), pregnancy zone protein, beta-2-glycoprotein 1, ceruloplasmin, serum paraoxonase/arylesterase 1, glutathione peroxidase 3, insulin-like growth factor-binding protein complex acid labile subunit, apolipoprotein C-Ill, albumin, apolipoprotein A-I, apolipoprotein A-IV, apolipoprotein E, complement factor I, hemopexin, plasminogen, and thrombospondin-1, or a fragment thereof.

The proteins can be directly attached to the HBPE nanoparticle or through a sequence that is directly attached to the HBPE nanoparticle. The one or more proteins can form a homogenous shell around the HBPE nanoparticle.

In some examples, the coronal protein-coated nanoparticle disclosed herein further comprises an anti-cancer therapeutic agent (for example, paclitaxel, docetaxel, or cabazitaxel) and/or an imaging compound.

Also disclosed herein is a cancer therapeutic composition comprising the coronal protein-coated nanoparticle disclosed herein.

Also disclosed herein is a method of treating cancer in a subject in need thereof, comprising administering to the subject a therapeutically effective amount of the coronal protein-coated nanoparticle disclosed herein.

Also disclosed herein is a method of generating one or more coronal protein-coated nanoparticles, comprising contacting one or more nanoparticles with a serum sample obtained from a subject, wherein the serum sample is obtained from a subject infected by influenza A virus, wherein the nanoparticle is a hyperbranched polyester (HBPE) nanoparticle.

### BRIEF DESCRIPTION OF THE DRAWINGS

The accompanying figures, which are incorporated in and constitute a part of this specification, illustrate several aspects described below.

FIG. 1 shows PEGylated HBPE-NPs accumulate in liver, spleen, and tumor, respectively. Shown are bar graphs for total organ fluorescence quantification (upper panel) and organ imaging (lower panel) 7 hours post-treatment of tumor-bearing mice intravenously injected with DiR dye-loaded PEGylated HBPE-PEG-NPs. Nu/Nu nude mouse was orthotopically injected with 8×10⁵ MDA-MB-231 TNBC cells in the mammary fat pad. Images were taken with an IVIS Lumina S5 and quantified with Living Image software. Images represent HBPE-PEG-NP uptake (DiR fluorescence) in the spleen, kidneys, lungs, heart, and tumor and liver.

FIGS. 2A-2D show biophysical characterization of HBPE polymer and NPs. FIGS. 2A-2B show HBPE polymer forms with correct branching a shown in inset. Hydrogen nuclear magnetic resonance (1H NMR) spectra of (FIG. 2A) monomer (compound 2) and (FIG. 2B) HBPE polymer. (FIG. 2C) Schematic of solvent diffusion method for production of HBPE-NPs and encapsulation of cargo. (FIG. 2D) TEM images of COOH-HBPE-NPs showing monodispered NPs and morphology. Images were acquired with a JEOL TEM-011 microscope.

FIGS. 3A-3B show HBPE-NPs that form sera-derived protein corona are non-toxic. (FIG. 3A) TEM images of NS-treated COOH-HBPE-NPs (left panel) and NS alone (right panel). NPs were incubated with sera in a 20:1 volumetric ratio for 15 minutes. Images were acquired with a JEOL TEM-011 microscope. (FIG. 3B) TNBC MDA-MB-231 cells (left panel), endothelial HUVECs (mid panel), and monocytic THP-1 cells (right panel) were treated with vehicle (water), COOH-HBPE-NPs and NS-coated COOH-HBPE-NPs for 24 hours of treatment. Cell viability was assessed using an MTT assay. Data in graph displays mean±standard deviation (n=3).

FIGS. 4A-4D show pre-treatment with NS improves cancer cell uptake of HBPE-NPs. (FIG. 4A) Representative confocal microscopy nanoparticle uptake images (single cell plane) of MDA-MB-231 cells (left column), HUVECs (middle column), and THP-1 cells (right column) treated with DiI dye-encapsulated COOH-HBPE-NPs (top row), PEG-HBPE-NPs (middle row), and NS-treated COOH-HBPE-NP (bottom row). Scale bar represents 50, 200, and 50 μm for MDA-MB-231, HUVEC, and THP-1 cells, respectively. Images were taken with a Zeiss LSM 710 microscope at 40× (MDA-MB-231 and THP-1 cells) and 20× (HUVECs) magnification. (FIGS. 4B-4D) Bar graphs represent average DiI fluorescence per cell. MDA-MB-231 cells (FIG. 4B), HUVECs (FIG. 4C) and THP-1 cells (FIG. 4D) were treated with COOH-HBPE-NP, PEG-HBPE-NPs, or NS-treated COOH-HBPE-NP (HS) for 24 hours. Imaging was performed with a Cytation 5 Cell Imaging Multi-Mode Reader (see FIG. 10 for representative images). MDA-MB-231 and HUVEC fluorescence data was acquired from 100 cells. THP-1 fluorescence data was acquired from a total of 50 cells. Fluorescence was quantified using ZEN blue software. Data represents mean±standard deviation. * p-value <0.0001 relative to PEG-HBPE-NPs. Representative data from three replicates is shown.

FIGS. 5A-5D show that NS-treated HBPE-NPs do not promote endothelial cell migration. (FIG. 5A) Schematic for CTEM protocol using the IncuCyte Live-Cell Analysis System. (FIG. 5B) HUVECs (Cyto-Light Green) were plated at 80% confluency and treated with DiI-encapsulated HBPE-NPs. Imaging was performed at 0.5-hour intervals for 24 hours detecting the green fluorescent (cells) and red fluorescent (nanoparticles) channels. Time course videos were made using the IncuCyte's chemotaxis software and select videographs analyzed for movement of red fluorescent particles. Red circles show the initial nanoparticle target site at time 0 and yellow circles show the location of the nanoparticle target after 20 hours. (FIGS. 5C-5D) Total green fluorescence graphs of HUVEC cells (Cyto-Light Green) treated with DiI-encapsulated COOH-HBPE-NPs (COOH-NPs), PEG-HBPE-NPs (PEG-NPs), and NS-treated HBPE-NPs are shown. Graphs track the migration of HUVEC cells above (FIG. 5C) or below (FIG. 5D) the insert through 48 hours (1-hour increments). (FIG. 5E) Representative top and bottom views of insert for fluorescent HUVEC cells at 18 hours post-treatment with PEG-NPs, HBPE-NPs or HBPE-NPs (NS).

FIGS. 6A-6E show that NS-treated HBPE-NPs are taken up by cancer cells after passage through endothelial layer. (FIG. 6A) Schematic for a transwell plate system consisting of a HUVEC-seeded insert and an MDA-MB-231-seeded bottom chamber. (FIG. 6B) Bar graph depicts optimization of HUVEC density. HUVECs were seeded at 60% confluency and proliferated through 8 days. At each timepoint, COOH-HBPE-NPs loaded with DiI were added to the top chamber and media from the bottom chamber collected for assessment of fluorescence using the Cytation 5 plate reader. (FIG. 6C) Bar graph depicts optimization of COOH-HBPE-NP treatment dose. HUVECs were seeded as in (FIG. 6B) and DiI-loaded COOH-HBPE-NPs added at increasing concentrations. After 24 hours, fluid from the bottom chamber was collected and total fluorescence determined as above. (FIG. 6D) Representative confocal microscopy images (single cell plane) of bottom chamber containing MDA-MB-231 cells. HUVECs were treated with DiI-encapsulated COOH-HBPE-NPs, PEG-HBPE-NPs, and NS-treated COOH-HBPE-NPs. Images of nanoparticle uptake by MDA-MB-231 cells were acquired 24 hours post-treatment. Scale bar represents 50 μm. Images were taken with a Zeiss LSM 710 microscope at 40× magnification. (FIG. 6E) Bar graph of digital images acquired from (FIG. 6D). Average DiI fluorescence per cell is shown (n=100 cells). Images were taken by a Cytation 5 Cell Imaging Multi-Mode Reader (see FIG. 11 for representative images). Data represents mean±standard deviation. *p-value <0.0001 relative to PEG-HBPE-NPs. Representative data from three replicates is shown.

FIGS. 7A-7B show increased killing of TNBC cells by taxol-loaded NS-HBPE-NPs. (FIG. 7A) MDA-MB-231 cells were treated with free taxol (50 nM or 43 μg), and COOH-HBPE-NPs, PEG-HBPE-NPs and NS-treated COOH-HBPE-NPs with or without taxol (0.01 mg polymer; 0.5-0.6 μg taxol) for 24 hours. Cell viability was assessed using an MTT assay. Data in graph displays mean±standard deviation (n=3). *p=0.0283, **p=0.0020. (FIG. 7B) Representative images of MDA-MB-231 cells treated as in (FIG. 7A) using the Cytation 5 Cell Imaging Multi-Mode Reader.

FIGS. 8A-8B show that COOH-HBPE-NPs and PEG-HBPE-NPs are equivalently loaded with cargo. NPs were treated with HCl-acidified 50 μl PBS solution (pH 4) to release cargo upon polymer degradation after hydrolysis. (FIG. 8A) Calibration curve was generated for serial dilutions of DiI as described in methods and read in a Cytation 5 multimodal plate reader at at 531 nm excitation and 593 nm emission wavelengths. Table summaries average fluorescence of DiI from 10 ug of hydrolyzed NPs in 50 ul volume and estimates DiI concentration in a typical 10 ul dose of HBPE-NPs. (FIG. 8B) Representative example of absorbance spectrum for NPs loaded with taxol and treated as above to release cargo. Absorbance was read at a 250 nm wavelength (UV/Vis) using a Beckman Coulter DU 800 Spectrophotometer. Tables show average absorbance for taxol released from 100 ug of NPs in 1 ml volume and the concentration of taxol estimated to be loaded in 0.01 mg polymer (in 1 ul) dose of HBPE-NPs based on the calibration curve generated from taxol serial dilutions.

FIG. 9 shows protein profiles associated with coronae formed on HBPE-NPs treated with normal mouse sera (NS). Proteins absorbed by COOHHBPE-NPs treated with NS was assessed by SDS-PAGE (sodium dodecyl sulfate polyacrylamide gel electrophoresis) gels and visualized by Coomassie staining. COOHHBPE-NPs were treated with sera at a volume:volume ratio of 5:1 or 20:1. A Precision-Plus Protein Dual Color protein reference ladder was used for molecular weight (MW) comparison.

FIG. 10 shows that HBPE-NPs pre-treated with NS show increased uptake by cancer cells. Representative Cytation 5 microscopic images taken 24 h post-treatment of MDA-MB-231 (left column), HUVEC (middle column), and THP-1 (right column) cells treated with DiI dye-encapsulated COOH-HBPE-NPs (top row), PEG-HBPE-NPs (middle row), and COOH-HBPE-NPs (normal sera, NS) (bottom row). Red fluorescence portrays nanoparticle uptake in cells (DiI dye presence). Scale bar represents 200 μm. Magnification was at 10×.

FIG. 11 shows HBPE-NPs pre-treated with NS show stable protein corona formation and uptake by breast cancer cells. Representative Cytation 5 microscopic images from the transwell (two-chamber) experiment. HUVECSs (upper chamber) were treated with DiI dye-encapsulated COOH-HBPE-NPs, HBPE-PEG-NPs, and NS-treated COOH-HBPE-NPs. The bottom chamber was seeded with MDA-MB-231 cells from which images of nanoparticle uptake were acquired. Red fluorescence portrays nanoparticle uptake by cells (DiI dye presence). Scale bar represents 200 μm. Magnification was at 10×.

FIGS. 12A-12D show that HBPE-NPs form IAV sera-derived protein corona and are non-toxic. (FIG. 12A) Scheme showing the process of IAV infection in C57BL/6 mice, correlating the phase of the immune response with weight loss. (FIGS. 12B-12D) MTT viability assay was performed to assess toxicity of sera coated NPs. MDA-MB-231 cells (FIG. 12B), HUVECs (FIG. 12C), and THP-1 cells (FIG. 12D) were treated with vehicle (water), COOH-HBPE-NPs (NPs) precoated with sera collected from day 3 post-IAV infection [NPs (VS3)], day 4 post-IAV infection [NPs (VS4)], day 5 post-IAV infection [NPs (VS5)], or day 6 post-IAV infection [NPs (VS6)]. Data represents mean±standard deviation (n=3).

FIGS. 13A-13D show that increased cancer cell uptake and decreased monocyte uptake is observed with HBPE-NPs pre-coated with sera collected from IAV-infected mice. (FIG. 13A) COOH-HBPE-NPs (NPs) pre-coated with VS3-6 was compared to PEG-HBPE-NPs (PEG-NPs) Representative confocal microscopic images (mid-plane) are shown of MDA-MB-231 cells (leftcolumn), HUVECs (middle column), and THP-1 cells (right column) cells treated with DiI dye-encapsulated PEG-HBPE-NPs (first row), NPs (VS3) (second row), NPs (VS4) (third row), NPs (VS5) (fourth row), and NPs (VS6) (fifth row). Red fluorescence is indicative of nanoparticle uptake in cells (DiI dye presence). Scale bar represents 50, 200, and 50 μm for MDA-MB-231 cells, HUVECs, and THP-1 cells, respectively. Images were taken with a Zeiss LSM 710 microscope at 40× (MDA-MB-231 and THP-1 cells) and 20× (HUVECs) magnification. (FIGS. 13B-13D) Red fluorescence (DiI nanoparticle presence) quantification bar graphs of (FIG. 13B) MDA-MB-231, (FIG. 13C) HUVEC and (FIG. 13D) THP-1 cells treated with PEG-NPs, NPs (VS3), NPs (VS4), NPs (VS5) or NPs (VS6) after 24 hours of treatment. Bar graphs represent average DiI fluorescence per cell. Quantification data originate from images taken by a Cytation 5 Cell Imaging Multi-Mode Reader (representative images in FIG. 21), in order to capture total nanoparticle fluorescence. MDA-MB-231 and HUVEC fluorescence data was acquired from a total of 100 cells. THP-1 fluorescence was acquired from a total of 50 cells. Fluorescence was quantified using Zen blue software. Data represents mean±standard deviation. * p-value <0.001, ** p value=0.0034 and *** p-value=0.0199 relative to PEG-NPs. Representative data from three replicates is shown.

FIG. 14 shows increased taxol-mediated toxicity of HBPE-NPs coated with IAV-infected mice sera toward triple-negative breast cancer cells. MTT viability assay was carried out to evaluate toxicity of taxol-loaded NPs (0.01 mg). MDA-MB-231 cells were treated with PBS vehicle, free taxol (50 nM/43 μg), PEG-HBPE-NPs, COOH-HBPE-NPs (V3), COOH-HBPE-NPs (V4), COOH-HBPE-NPs (V5), or COOH-HBPE-NPs (V6). Data represents mean±standard deviation (n=3). *p-value <0.05 relative to PEG-HBPE-NPs.

FIGS. 15A-15B show that IAV-treated HBPE-NPs are taken up by cancer cells after passing through the endothelial layer. (FIG. 15A) Transwell experiment in which HUVECs (top chamber) were treated with DiI-loaded HBPE-NPs and uptake was assessed by MDA-MB-231 cells (bottom chamber). Representative confocal microscopy images of MDA-MB-231 cells are shown after 24-hour treatment of HUVECs with PEG-HBPE-NPs (PEG-NPs), COOH-HBPE-NPs (NPs (VS3)), NPs (VS4), NPs (VS5), and NPs (VS6). Scale bar represents 50 μm. Images were acquired at 40× magnification. (FIG. 15B) Red fluorescence (DiI nanoparticle presence) quantification bar graphs of bottom chamber MDA-MB-231 cells 24-hour post-treatment of top chamber HUVECs with PEG-NPs, NPs (VS3), NPs (VS4), NPs (VS5), or NPs (VS6). Average DiI fluorescence per cell was used for quantification from images of 100 cells taken by a Cytation 5 Cell Imaging Multi-Mode Reader. Fluorescence was quantified using Zen blue software. Data represents mean±standard deviation. *p-value <0.0001, **p-value=0.0002, and ***p-value=0.0028 relative to PEG-NPs. Representative experiments from three replicates are shown.

FIGS. 16A-16B show biodistribution of HBPE-NPs in mice is modulated by formation of coronae. Nu/Nu nude mice were orthotopically injected with 8×10⁵ MDA-MB-231 triple-negative breast cancer cells in the mammary fat pad. Upon tumors reaching 1000 mm³, mice were intravenously injected with DiR dye-encapsulated PEG-HBPE-NPs (PEG-NPs), PEG-NPs (VS5), COOH-HBPE-NPs (NPs), and NPs (VS5) and imaged 7 hours post-treatment. Images were acquired with an IVIS Lumina S5 and quantified with Living Image software. (FIG. 16A) Total organ fluorescence (nanoparticle presence) quantification bar graphs (upper panels) and (FIG. 16B) organ images (lower panels). Images represent nanoparticle uptake (DiR fluorescence) in the tumor, heart, lungs, spleen, kidneys, and liver. Blue color constitutes low nanoparticle accumulation and red/yellow color constitutes high nanoparticle accumulation. Data represents mean±standard deviation (n=3). *p-value <0.0001, **p-value=0.0043, ***p-value=0.0156, and ****p-value=0.0185 relative to NPs.

FIGS. 17A-17D show that unique protein profiles are associated with coronae formed on HBPE-NPs treated with sera from collected from IAV-infected mice. Proteins absorbed by (PEG) or COOH-HBPE-NPs (NP) treated with VS3-6 was assessed by SDS-PAGE (sodium dodecyl sulfate polyacrylamide gel electrophoresis) gels and visualized by Coomassie staining. COOH-HBPE-NPs were treated with sera at a volume:volume ratio of (FIG. 17A) 5:1 or (FIG. 17B) 20:1. A Precision-Plus Protein Dual Color protein reference ladder was used for molecular weight (MW) comparison. Histograms of individual lane patterns are shown for the whole lane, upper third of lane, or lower third of lane for 5:1 (FIG. 17C) and 20:1 (FIG. 17D) ratio gels. Two peaks (1, 2) were selected for quantitation and ratios of peak 1 to 2 is shown. * Indicated unique regions of interest in each lane. Histograms were created and quantified through ImageJ gel analysis software.

FIGS. 18A-18B show that TSP-1 is associated with coronae formed on HBPE-NPs. Proteins absorbed by COOH-HBPE-NPs (NP) treated with VS3 and VS5 was assessed by SDS-PAGE (sodium dodecyl sulfate polyacrylamide gel electrophoresis) gels. (FIG. 18A) TSP-1 presence in sera alone or on NPs were determined using a TSP-1 antibody (upper panel) and antibody signal normalized to total protein was quantified using Licor Image Studio Lite software (lower panel). (FIG. 18B) Total protein per lane was evaluated using REVERT total protein staining. COOH-HBPE-NPs were treated with sera at a volume:volume ratio of 20:1. A Precision-Plus Protein Dual Color protein reference ladder was used for molecular weight (MW) comparison. Abbreviations: TSP-1 (thrombospondin-1.

FIG. 19 shows immune-related sheddome. Known or predicted shed proteins related to the immune system, organized by category. Frequency of proteins (n=127) within each category are represented as a percentage. Proteins were identified through SheddomeDB or DeepSMP (A Deep Learning Model for Predicting the Shedding Events of Membrane Proteins) databases.

FIG. 20 shows that HBPE-NPs pre-coated with sera collected from mice infected with IAV display improved cancer cell uptake and reduce monocyte uptake over HBPE-PEG-NPs. Uptake of COOH-HBPE-NPs (NPs) were coated with influenza A virus (IAV)-infected mouse sera (days 3 to 6 of infection) was compared to PEG-HBPE-NPs (PEG-NPs) uptake. Representative Cytation 5 microscopy nanoparticle uptake images 24 h post-treatment of MDA-MB-231 (left column), HUVEC (middle column), and THP-1 (right column) cells treated with DiI dye-encapsulated PEG-NPs (topmost row), NPs (VS3) (second row), NPs (VS4) (third row), NPs (VS5) (fourth row), and NPs (VS6) (fifth row). Red fluorescence depicts nanoparticle uptake in cells (DiI dye presence). Scale bar represents 200 μm. Magnification was at 10×.

FIGS. 21A-21D show that HBPE-NPs pre-coated with sera collected from IAV-infected mice do not promote the migration of endothelial cells. Employing the Incucyte Live-Cell analysis system, HUVEC cells (Cytolight Green) total green fluorescence graphs are shown (A) above or (B) below pore filters for 48 hours (1 hour increments) of treatment with DiI-encapsulated PEG-HBPE-NP (PEG-NPs), COOH-HBPE-NP (VS3) (HBPE-NP (VS3), HBPE-NP (VS4), HBPE-NP (VS5), and HBPE-NP (VS6). (C) Representative HUVEC fluorescent images at 18 hours post-treatment with PEG-NPs, HBPE-NPs (VS3), HBPE-NPs (VS4), HBPE-NPs (VS5), and HBPE-NPs (VS6). Magnification was at 10×. (D) Incucyte Live-Cell analysis system was used to quantify total red fluorescence count signal of DiI-loaded PEG-NPs, HBPE-NPs (VS3), HBPE-NPs (VS4), HBPE-NPs (VS5), HBPE-NPs (VS6) after 24 hours (30 min increments) of treatment with GFP-HUVEC cells. Data represents mean total RFP (HBPE-NPs) or GFP (HUVECs) count per well.

FIG. 22 shows that HBPE-NPs pre-coated with sera collected from IAV-infected mice reveal improved migration and cancer cell uptake. Uptake of COOH-HBPE-NPs (NPs) that were coated with sera from influenza A virus (IAV)-infected mouse sera (days 3 to 6 of infection) was compared to HBPE-PEG-NPs (PEG-NPs). Representative Cytation 5 microscopy images were taken 24 hour post-treatment of HUVECs (top chamber) with DiI dye-encapsulated NPs and uptake visualized by imaging MDA-MB-231 cells (bottom chamber). Panels are PEG-NPs (topmost panel), NPs (VS3) (second panel), NPs (VS4) (third panel), NPs (VS5) (fourth panel), and NPs (VS6) (fifth panel). Red fluorescence depicts nanoparticle uptake in cells (DiI dye presence). Scale bar represents 200 μm. Magnification was at 10×.

FIGS. 23A-23B show synthesis schematic for HBPE polymer. FIG. 23A shows that BBA, DEM, and K2CO3 reactants were dissolved in acetonitrile in molar ratios of 1, 1.1, and 1, respectively. After 36 hours under reflux, 2-(4-Acetoxybutyl) malonic acid diethyl ether product (compound 1) was purified by separatory funnel extraction, rotary evaporation, and vacuum distillation. Oxygen-bound end groups were deprotected with NaOH and protonated with HCl. The monomer product (compound 2) was purified through vacuum distillation and rotary evaporation. The monomer was then polymerized with a (PTSA) acid catalyst in a DMSO solvent under inert nitrogen atmosphere. For polymerization, the monomer was dispensed in solution with a syringe pump at a 0.1 mL per hour rate. FIG. 23B shows that, for seed-based polymerization, all synthesis steps were identical to non-seed HBPE polymerization apart from the following. After compound 2 purification, the monomer was then polymerized with PTSA catalyst and a terephthalic acid seed in DMSO solvent under inert nitrogen atmosphere.

FIGS. 24A-24B show effect of pre-coating HBPE-NPs with 6 fold diluted sera collected from IAV-infected mice, which in reduced cancer cell uptake compared to HBPE-NPs pre-coated with undiluted IAV-infected mice sera as described in FIG. 3A-B. Uptake of HBPE-NPs (NPs) coated with undiluted (u) sera from influenza A virus (IAV)-infected mouse sera (day 5 of infection)(V5) were compared to NPs coated with diluted 6 fold (d) IAV-infected mouse V5 sera. NPs were incubated in sera for 0.5 h or 4 h. Representative Cytation 5 microscopy NP uptake images were taken 24 hour post-treatment of MDA-MB-231s with DiI dye-encapsulated NPs (FIG. 24A). Panels include NPs incubated in undiluted sera for 0.5 hr (upper panel), NPs incubated in diluted sera for 0.5 hr (middle panel), and NPs incubated in diluted sera for 4 hr (lower panel). Red fluorescence depicts nanoparticle uptake in cells (DiI dye presence). Scale bar represents 200 μm. Magnification was at 10×. Bar graphs show quantification of uptake of NPs by MDA-MB-231s, after 24 hours of treatment FIG. 24B). NPs were incubated with undiluted or diluted V5 sera for 0.5 h or 4 h. Bar graphs represent average DiI fluorescence per cell. Quantification data originated from images (A) taken by a Cytation 5 Cell Imaging Multi-Mode Reader, representing total nanoparticle fluorescence. Quantification data was acquired from a total of 100 cells. Fluorescence was quantified using Zen blue software. Data represents mean±standard deviation. * p-value <0.001 relative to NP (V5)(u) 0.5 h.

### DETAILED DESCRIPTION

The disclosed subject matter can be understood more readily by reference to the following detailed description, the Figures, and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is understood that the disclosed methods and systems are not limited to the particular methodology, protocols, and systems described as these may vary. It is also to be understood that the terminology used herein is for the purpose of describing particular embodiments only, and is not intended to limit the scope of the present invention which will be limited only by the appended claims.

Terms used throughout this application are to be construed with ordinary and typical meaning to those of ordinary skill in the art. However, Applicant desires that the following terms be given the particular definition as defined below.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

The term “about” as used herein when referring to a measurable value such as an amount, a percentage, and the like, is meant to encompass variations of ±20%, ±10%, ±5%, or ±1% from the measurable value.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the amino acid abbreviations are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “biocompatible” generally refers to a material and any metabolites or degradation products thereof that are generally non-toxic to the recipient and do not cause significant adverse effects to the subject.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc.

Contacting: Placement in direct physical association, for example solid, liquid or gaseous forms. Contacting includes, for example, direct physical association of fully- and partially-solvated molecules.

As used herein, the term “cancer” refers to a proliferative disorder or disease caused or characterized by the proliferation of cells which have lost susceptibility to normal growth control. The term “cancer” includes tumors and any other proliferative disorders. Cancers of the same tissue type originate in the same tissue, and can be divided into different subtypes based on their biological characteristics. Cancer includes, but is not limited to, melanoma, leukemia, astrocytoma, glioblastoma, lymphoma, glioma, Hodgkin's lymphoma, and chronic lymphocyte leukemia. Cancer also includes, but is not limited to, cancer of the brain, bone, pancreas, lung, liver, breast, thyroid, ovary, uterus, testis, pituitary, kidney, stomach, esophagus, anus, and rectum.

The term “cancer cells” and “tumor cells” are used interchangeably to refer to cells derived from a cancer or a tumor, or from a tumor cell line or a tumor cell culture.

The term “primary tumor” refers to a tumor growing at the site of the cancer origin.

The term “metastatic tumor” refers to a secondary tumor growing at the site different from the site of the cancer origin.

A “control” is an alternative subject or sample used in an experiment for comparison purposes. A control can be “positive” or “negative.”

As used herein, the term “diagnosed” means having been subjected to a physical examination by a person of skill, for example, a physician or a researcher, and found to have a condition that can be diagnosed or treated by compositions or methods disclosed herein. For example, “diagnosed with cancer” means having been subjected to a physical examination by a person of skill, for example, a physician or a researcher, and found to have a condition that can be diagnosed or treated by a compound or composition that alleviates or ameliorates cancer and/or aberrant cell growth.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property.

The term “increased” or “increase” as used herein generally means an increase by a statically significant amount; for the avoidance of any doubt, “increased” means an increase of at least 5% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level so long as the increase is statistically significant.

As used herein, the term “ligand” refers to a biomolecule or a chemical entity having a capacity or affinity for binding to a target. A ligand can include many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof (e.g., Fab, scFv), a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others. A ligand can come from many sources, including libraries, such as small molecule libraries, phage display libraries, aptamer libraries, or any other library as would be apparent to one of ordinary skill in the art after review of the disclosure of the present disclosure. In some embodiments, the ligand can be a small molecule or a polypeptide that specifically binds to a receptor on an intestinal endothelial cell.

As used herein, the term “metastasis” is meant to refer to the process in which cancer cells originating in one organ or part of the body, with or without transit by a body fluid, and relocate to another part of the body and continue to replicate. Metastasized cells can subsequently form tumors which may further metastasize. Metastasis thus refers to the spread of cancer, from the part of the body where it originally occurred, to other parts of the body.

As used herein, “noncancerous cells” and “noncancerous tissue” can refer to cells or tissue, respectively, that are normal or cells or tissue that do not exhibit any metabolic or physiological characteristics associated with cancer. For example, noncancerous cells and noncancerous tissues are healthy and normal cells and tissues, respectively.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, P A, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.10% and 99% by weight of the total of one or more of the subject compounds based on the weight of the total composition including carrier or diluent.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e. is biologically inert and physiologically acceptable, non-toxic, and is biodegradable in the environment of use, i.e. can be resorbed by the body. The term “polymer” encompasses all forms of polymers including, but not limited to, natural polymers, synthetic polymers, homopolymers, heteropolymers or copolymers, addition polymers, etc.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level so long as the decrease is statistically significant.

In the present invention, “specific for” and “specificity” mean selective binding. The term “binding,” and “specific binding” are used interchangeably to refer to the ability of a reagent to selectively bind its target. In some examples, specificity is characterized by a dissociation constant of 10⁴ M⁻¹ to 10¹² M⁻¹. Empirical methods using appropriate controls may be employed to distinguish specific and non-specific binding in a particular case.

The term “subject” refers to a human in need of treatment for any purpose, and more preferably a human in need of treatment (e.g., to treat cancer). The term “subject” can also refer to non-human animals, such as non-human primates.

As used herein, a “target”, “target molecule”, or “target cell” refers to a biomolecule or a cell that can be the focus of a therapeutic drug strategy, diagnostic assay, or a combination thereof, sometimes referred to as a theranostic. Therefore, a target can include, without limitation, many organic molecules that can be produced by a living organism or synthesized, for example, a protein or portion thereof, a peptide, a polysaccharide, an oligosaccharide, a sugar, a glycoprotein, a lipid, a phospholipid, a polynucleotide or portion thereof, an oligonucleotide, an aptamer, a nucleotide, a nucleoside, DNA, RNA, a DNA/RNA chimera, an antibody or fragment thereof, a receptor or a fragment thereof, a receptor ligand, a nucleic acid-protein fusion, a hapten, a nucleic acid, a virus or a portion thereof, an enzyme, a co-factor, a cytokine, a chemokine, as well as small molecules (e.g., a chemical compound), for example, primary metabolites, secondary metabolites, and other biological or chemical molecules that are capable of activating, inhibiting, or modulating a biochemical pathway or process, and/or any other affinity agent, among others.

As used herein, the terms “treating” or “treatment” of a subject includes the administration of a drug to a subject with the purpose of curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, and improvement or remediation of damage.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of cancer, or a symptom of cancer. In some embodiments, a desired therapeutic result is the control of metastasis. In some embodiments, a desired therapeutic result is the prevention or control of relapse. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “variant” as used herein refers to a polypeptide or polynucleotide that differs from a reference polypeptide or polynucleotide, but retains essential properties. A typical variant of a polypeptide differs in amino acid sequence from another, reference, polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions).

Disclosed are the components to be used to prepare a composition disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds can not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions disclosed herein. Thus, if there are a variety of additional steps that can be performed it is understood that each of these additional steps can be performed with any specific embodiment or combination of embodiments of the methods disclosed herein.

### Nanoparticles

Disclosed herein are nanoparticles. Nanoparticles (NPs) have been used for drug delivery or vaccines. However, optimizing a nanoparticle's biological identity for therapeutic benefit remains challenging. Macromolecules absorption from biofluids by nanoparticles forms a layer called the “protein corona”. The macromolecule of the protein corona is also termed herein as “coronal protein”. Accordingly, in some aspects, disclosed herein are coronal protein-coated nanoparticles and the uses thereof.

In some aspects, disclosed herein are coronal protein-coated nanoparticle comprising one or more proteins. The nanoparticle used herein can be any nanoparticle useful for the delivery of polypeptides. The term “nanoparticle” as used herein refers to a particle or structure which is biocompatible with and sufficiently resistant to chemical and/or physical destruction by the environment of such use so that a sufficient number of the nanoparticles remain substantially intact after delivery to the site of application or treatment and whose size is in the nanometer range. In some embodiments, the nanoparticles are those described in International Publication Nos. WO2013056132, WO2016187531A1, WO2017176974, and WO2019027999; U.S. Pat. No. 10,143,660; and U.S. Application Publication No. 2013/0216807; which are incorporated herein by reference in their entireties.

In some examples, the nanoparticles are hyberbranched polyester polymeric nanoparticles (HBPE-NPs or just HBPE). In an aspect, the nanoparticles are polymeric nanoparticles. In some examples, the nanoparticle comprises a hydrophobic core and a hydrophilic shell. In some examples, the hydrophilic shell comprises a carboxylic acid group. In some examples, the HBPE nanoparticles are those described in U.S. Pat. No. 10,973,925, which is incorporated herein by reference in its entirety. The coronal protein-coated nanoparticle disclosed herein comprises one or more proteins, wherein the one or more proteins are selected from the group consisting of Albumin, Complement C3, Pregnancy zone protein, Apolipoprotein A-I, Cluster of GLOBIN domain-containing protein, GLOBIN domain-containing protein, Serotransferrin, Ceruloplasmin, Cluster of Murinoglobulin-1, Murinoglobulin-1, Hemopexin, Cluster of Serine protease inhibitor A3K, Serine protease inhibitor A3K, Kininogen-1, Hemoglobin subunit beta-2, Plasminogen, Haptoglobin, Cluster of Alpha-1-antitrypsin 1-4, Alpha-2-HS-glycoprotein, Thrombospondin-1, Hemoglobin subunit alpha, Alpha-1-antitrypsin 1-4, Murinoglobulin-2, Alpha-1-antitrypsin 1-1, Alpha-1-antitrypsin 1-2, Inter alpha-trypsin inhibitor, heavy chain 4, Fibronectin, Vitamin D-binding protein, Prothrombin, Spectrin beta chain, erythrocytic, Serine protease inhibitor A3M, Cluster of Complement factor H, Complement factor H, Apolipoprotein E, Apolipoprotein A-IV, Cluster of Carboxylesterase 1C, Complement factor B, Beta-2-glycoprotein 1, Cluster of Ankyrin-1, Ankyrin-1, Spectrin alpha chain, erythrocytic 1, Carboxylesterase 1C, Apolipoprotein B-100, Serine protease inhibitor A3N, Inhibitor of carbonic anhydrase, Complement factor I, Complement C4-B, Histidine-rich glycoprotein, Cluster of Actin, cytoplasmic 1, Actin, cytoplasmic 1, Afamin, Inter-alpha-trypsin inhibitor heavy chain H2, Protein AMBP, Actin, alpha cardiac muscle 1, Inter-alpha-trypsin inhibitor heavy chain H3, LRRCT domain-containing protein, Vitronectin, Clusterin, Phosphatidylinositol-glycan-specific phospholipase D, Plasma kallikrein, Complement C5, Cluster of Keratin, type II cytoskeletal 2 epidermal, Cluster of H-2 class I histocompatibility antigen, Q10 alpha chain, H-2 class I histocompatibility antigen, Q10 alpha chain, Band 3 anion transport protein, Carboxylic ester hydrolase, Immunoglobulin heavy constant mu, Inter-alpha-trypsin inhibitor heavy chain H1, Serum amyloid P-component, Apolipoprotein M, Beta-actin-like protein 2, Gelsolin, Antithrombin-III, Alpha-1B-glycoprotein, CD5 antigen-like, Sulfhydryl oxidase 1, HMW kininogen-II, Ig-like domain-containing protein, Fetuin-B, Properdin, Ig gamma-2B chain C region, Complement component C8 alpha chain, Cluster of Heat shock cognate 71 kDa protein, Serum amyloid A-1 protein, Immunoglobulin kappa constant, Glutathione peroxidase 3, Heat shock cognate 71 kDa protein, Alpha-1-antitrypsin 1-5, Apolipoprotein A-II, Talin-1, Coagulation factor V, Carboxypeptidase N subunit 2, Fibrinogen alpha chain, Ig-like domain-containing protein, Coagulation factor X, Immunoglobulin heavy constant gamma 3, Carbonic anhydrase 1, Keratin, type II cytoskeletal 5, Apolipoprotein D, Ig gamma-1 chain C region, membrane-bound form, Apolipoprotein C-IV, Alpha-2-antiplasmin, Beta-2-microglobulin, Immunoglobulin heavy constant gamma 2C, Alpha-1-acid glycoprotein 1, Complement component C8 beta chain, N-acetylmuramoyl-L-alanine amidase, Vitamin K-dependent protein Z, Cluster of Filamin-A, Filamin-A, BPI fold-containing family A member 2, Serum paraoxonase/arylesterase 1, Serum amyloid A-4 protein, Epidermal growth factor receptor, Zinc-alpha-2-glycoprotein, Phosphatidylcholine-sterol acyltransferase, Mannose-binding protein C, Carboxypeptidase N catalytic chain, SCY domain-containing protein, Interleukin-1 receptor accessory protein, Immunoglobulin J chain, Complement C1q subcomponent subunit A, Keratin, type II cytoskeletal 2 epidermal, Keratin, type II cytoskeletal 1, IF rod domain-containing protein, Keratin, type II cytoskeletal 1b, Keratin, type II cytoskeletal 75, Carboxylic ester hydrolase, Fibrinogen gamma chain, Cluster of Keratin, type I cytoskeletal 10, Keratin, type I cytoskeletal 10, Serum amyloid A-2 protein, Transthyretin, Carbonic anhydrase 2, Immunoglobulin heavy constant alpha, Plasma protease C1 inhibitor, Transitional endoplasmic reticulum ATPase, Coagulation factor XII, Glyceraldehyde-3-phosphate dehydrogenase, Mannan-binding lectin serine protease 2, Alpha-1-acid glycoprotein 2, sp|Q8BXA7|PHLP2_DECOY, Keratin, type II cytoskeletal 73, IF rod domain-containing protein, Keratin, type II cytoskeletal 2 oral, Apolipoprotein C-III, Predicted gene 4788, Endoplasmic reticulum chaperone BiP, IF rod domain-containing protein, Selenoprotein P, Platelet-activating factor acetylhydrolase, Complement component C9, sp|P46656|ADX_DECOY, Corticosteroid-binding globulin, Platelet factor 4, Cluster of Alpha-amylase 1, Alpha-amylase 1, Heparin cofactor 2, Ig-like domain-containing protein, Cluster of Tubulin alpha-4A chain, Tubulin alpha-4A chain, Complement factor D, Retinol-binding protein 4, 14-3-3 protein zeta/delta, Secreted phosphoprotein 24, Macrophage colony-stimulating factor 1 receptor, Lumican, 55 kDa erythrocyte membrane protein, sp|Q9WTU0|PHF2 DECOY, sp|P59242|CING_DECOY, C-reactive protein, Coagulation factor IX, Peroxiredoxin-2, Vitamin K-dependent protein S, sp|Q571H0|NPA1P_DECOY, Ficolin-1, Ig lambda-2 chain C region, Profilin-1, Extracellular superoxide dismutase [Cu—Zn], Complement C1q subcomponent subunit C, Biotinidase, Keratin, type II cytoskeletal 79, Keratin, type II cytoskeletal 8, Keratin, type II cytoskeletal 74, Ankyrin-3, Ankyrin-2, C4b-binding protein, Apolipoprotein C-I, Keratin, type I cytoskeletal 16, Pigment epithelium-derived factor, Endogenous retroviral sequence 3, Predicted gene 382, Cluster of Myosin-9, Myosin-9, Myosin-11, sp|P98083|SHC1_DECOY, Hepatocyte growth factor activator, Pancreatic alpha-amylase, Aamy domain-containing protein, Ig-like domain-containing protein, Keratin, type I cytoskeletal 28, Spectrin alpha chain, non-erythrocytic 1, Extracellular matrix protein 1, Fibrinogen beta chain, Mannose-binding protein A, Carboxypeptidase B2, Tubulin alpha-1B chain, Complement component C8 gamma chain, Keratin, type I cytoskeletal 42, Protein PRRC2B, Lysosomal alpha-mann, OSidase, sp|Q6RUT8|CC154_DECOY, sp|Q8BMD6|TM260_DECOY, Ras-related protein Rap-1b, Alpha-actinin-1, Proteoglycan 4, Ig-like domain-containing protein, von Willebrand factor, Serglycin, sp|A2AAY5|SPD2B_DECOY, sp|Q9JLV2|TP4AP_DECOY, Hyaluronan-binding protein 2, Disks large homolog 5, sp|Q7TSJ2|MAP6_DECOY, Amyloid-beta A4 precursor protein-binding family B member 2, sp|Q9Z1P8|ANGL4_DECOY, tr|J3QNP2|J3QNP2_DECOY, Insulin-like growth factor-binding protein complex acid labile subunit, Fermitin family homolog 3, Ig-like domain-containing protein, Ig kappa chain V-II region 26-10, Ig kappa chain V-V region MOPC 41, L-selectin, Ig kappa chain V-VI region XRPC 44, Leukemia inhibitory factor receptor, Platelet glycoprotein Ib alpha chain, Hepatocyte growth factor-like protein, Nuclear receptor subfamily 1 group D member 2, sp|B2RQE8|RHG42_DECOY, Lysozyme C-2, Tubulin alpha-8 chain, Vitamin K-dependent protein C, RIKEN cDNA 9530053A07 gene, Ig-like domain-containing protein, Ig-like domain-containing protein, Superoxide dismutase [Cu—Zn], Triosephosphate isomerase, Anaphylatoxin-like domain-containing protein, Hepcidin, Peptidyl-prolyl cis-trans isomerase A, Metalloproteinase inhibitor 3, Ig-like domain-containing protein, Immunoglobulin heavy variable 1-15, Progranulin, Testisin, tr|Q91X36|Q91X36_DECOY, sp|Q8VEE4|RFA1_DECOY, Complement C1r-A subcomponent, Carboxypeptidase Q, Dematin, Ig heavy chain V region MOPC 47A, T-lymphoma invasion and metastasis-inducing protein 1, Ras-related protein Rab-19, Protein disulfide-isomerase A3, Proto-oncogene tyrosine-protein kinase receptor Ret, Ig-like domain-containing protein, Monocarboxylate transporter 1, sp|P67984|RL22_DECOY, Phosphatidylinositide phosphatase SAC2, Complement factor H-related 2, Serine protease inhibitor A3G, Neurofilament heavy polypeptide, H-2 class I histocompatibility antigen, alpha chain, H-2 class I histocompatibility antigen, Q8 alpha chain, Ig-like domain-containing protein, Filamin-B, Filamin-C, sp|A2ASS6|TITIN_DECOY, phospholipid transfer protein, sp|Q6ZWQ0|SYNE2_DECOY, Desmoplakin, Pericentrin, sp|Q80SU7|GVIN1 DECOY, Complement component 6, Transmembrane protein KIAA1109, sp|Q3UHF7|ZEP2_DECOY, sp|A2BH40|ARI1A_DECOY, E3 ubiquitin-protein ligase MIB1, tr|B1AR51|B1AR51_DECOY, Cluster of Tubulin beta-4B chain, Tubulin beta-4B chain, Tubulin beta-3 chain, Tubulin beta-2A chain, sp|Q8R3B7|BRD8_DECOY, sp|Q6A0781CE290_DECOY, sp|Q66JQ7|KNL1_DECOY, sp|A2ARZ3|FSIP2_DECOY, Myosin light polypeptide 6, Cathelicidin antimicrobial peptide, Tubulin beta-1 chain, Keratin, type I cytoskeletal 13, sp|Q9R269|PEPL_DECOY, PDZ domain-containing protein, sp|Q61285|ABCD2 DECOY, RIKEN cDNA 4930407110 gene, Cluster of sp|A2A432|CUL4B_DECOY, sp|A2A432|CUL4B_DECOY, sp|Q3TCH7|CUL4A_DECOY, Cystatin domain-containing protein, VWFA domain-containing protein, sp|Q60988|STIL_DECOY, sp|Q8WTY4|CPIN1_DECOY, Klotho, sp|Q9R1L5|MAST1_DECOY, Vasculin-like protein 1, Cluster of Ig kappa chain V-V region HP R16.7, Ig kappa chain V-V region HP R16.7, Ig kappa chain V-V region HP 123E6, Pyruvate kinase PKM, Bridging integrator 2, Angiotensinogen, Peptidase Si domain-containing protein, Apolipoprotein N, tr|B2RPU8|B2RPU8_DECOY, Histone-lysine N-methyltransferase 2D, DNA topoisomerase 3-alpha, Cysteine and glycine-rich protein 1, Vascular cell adhesion protein 1, Apolipoprotein A-V, Flavin reductase (NADPH), Dual specificity protein phosphatase 3, Peptidase Si domain-containing protein, Ras-related protein Rab-8B, Nucleolar and coiled-body phosphoprotein 1, sp|B2RPV6|MMRN1_DECOY, Proprotein convertase subtilisin/kexin type 9, sp|P97350|PKP1_DECOY, Isoleucine-tRNA ligase, mitochondrial, sp|Q7TQC5|APTX_DECOY, sp|O88329|MYO1A_DECOY, sp|Q9D920|BORC5_DECOY, Cilia- and flagella-associated protein 221, Ig-like domain-containing protein, Ig kappa chain V-III region PC 3741/TEPC 111, Ig-like domain-containing protein, Ig-like domain-containing protein, Ig-like domain-containing protein, Ig-like domain-containing protein, Ig-like domain-containing protein, Angiopoietin-1, Cofilin-1, EH domain-containing protein 4, Ras-related C3 botulinum toxin substrate 1, Basigin, SPARC-like protein 1, sp|Q8K370|ACD10_DECOY, sp|Q99N80|SYTL1_DECOY, Mannan-binding lectin serine protease 1, BPI fold-containing family A member 1, Cardiotrophin-like cytokine factor 1, A disintegrin and metallopeptidase domain 6B, Polypeptide N-acetylgalactosaminyltransferase 13, Coagulation factor XIII B chain, Antileukoproteinase, tr|E9Q6R1|E9Q6R1 DECOY, Proteasome subunit alpha type-2, sp|Q8CFS6|KCNV2_DECOY, sp|O08550|KMT2B_DECOY, and Discoidin domain-containing receptor 2, a fragment thereof, or a variant thereof. In some examples, the one or more proteins are selected from those in Table 7. The one or more proteins can form a homogenous shell around the HBPE nanoparticle.

In some examples, the one or more proteins are selected from the group consisting of complement C3, alpha-2-HS-glycoprotein, complement factor B, vitronectin, clusterin, inhibitor of carbonic anhydrase, H-2 class I histocompatibility antigen, Q10 alpha chain, complement C5, carboxypeptidase N subunit 2, plasma protease C1 inhibitor, alpha-1-acid glycoprotein 1, alpha-2-antiplasmin, complement component C8 alpha chain, complement component C9, serum amyloid A-1 protein, complement factor D, serum amyloid A-2 protein, Ig-like domain-containing protein, complement C1s-A subcomponent, N-acetylmuramoyl-L-alanine amidase, carboxypeptidase N catalytic chain, complement C2, complement component 7, mannan-binding lectin serine protease 2, ficolin-1, complement C1r-A subcomponent, vitamin K-dependent protein S, mannan-binding lectin serine protease 1, glyceraldehyde-3-phosphate dehydrogenase, vitamin K-dependent protein C, interleukin-1 receptor accessory protein, fibronectin, apolipoprotein B-100, complement factor H, haptoglobin, immunoglobulin heavy constant mu, complement component C8 beta chain, Ig gamma-2B chain C region, protein AMBP, Ig gamma-1 chain C region, membrane-bound form, complement component C8 gamma chain, alpha-1-acid glycoprotein 2, immunoglobulin kappa constant, mannose-binding protein C, beta-2-microglobulin, serum amyloid P-component, complement C1s-B subcomponent, transthyretin, inter alpha-trypsin inhibitor, heavy chain 4, Inter-alpha-trypsin inhibitor heavy chain H2, histidine-rich glycoprotein, afamin, apolipoprotein A-II, corticosteroid-binding globulin, flavin reductase (NADPH), pregnancy zone protein, beta-2-glycoprotein 1, ceruloplasmin, serum paraoxonase/arylesterase 1, glutathione peroxidase 3, insulin-like growth factor-binding protein complex acid labile subunit, apolipoprotein C-III, albumin, apolipoprotein A-I, apolipoprotein A-IV, apolipoprotein E, complement factor I, hemopexin, plasminogen, and thrombospondin-1, or a fragment thereof.

In some examples, the one or more proteins are selected from the group consisting of inter alpha-trypsin inhibitor, heavy chain 4, alpha-2-HS-glycoprotein, inter-alpha-trypsin inhibitor heavy chain H2, clusterin, histidine-rich glycoprotein, afamin, carboxypeptidase N subunit 2, apolipoprotein A-II, corticosteroid-binding globulin, and flavin reductase (NADPH), or a fragment thereof.

In some examples, the one or more proteins are selected from the group consisting of pregnancy zone protein, apolipoprotein B-100, beta-2-glycoprotein 1, ceruloplasmin, serum paraoxonase/arylesterase 1, glutathione peroxidase 3, insulin-like growth factor-binding protein complex acid labile subunit, apolipoprotein C-III, beta-2-microglobulin, and mannose-binding protein C, or a fragment thereof.

In some examples, the one or more proteins are selected from the group consisting of albumin, alpha-2-HS-glyco protein, apolipoprotein A-I, apolipoprotein A-IV, apolipoprotein B-100, apolipoprotein E, beta-2-glycoprotein 1, ceruloplasmin, clusterin, complement factor I, hemopexin, histidine-rich glycoprotein, inter alpha-trypsin inhibitor, heavy chain 4, plasminogen, pregnancy zone protein, and thrombospondin-1, or a fragment thereof.

In some examples, the coronal protein-coated nanoparticle disclosed herein comprises thrombospondin-1 or a fragment thereof.

In some examples, the coronal proteins include, for example, Complement C3 (NCBI accession number: NP_000055), Alpha-2-HS-glycoprotein (NCBI accession number: AAA51683), Complement factor B (NCBI accession number: CAA51389), Vitronectin (NCBI accession number: NP_000629), Clusterin (NCBI accession number: NP_001822), Complement C5 (NCBI accession number: NP_001726), Carboxypeptidase N subunit 2 (NCBI accession number: NP_001278917), Plasma protease C1 inhibitor (NCBI accession number: NP_001027466), Alpha-1-acid glycoprotein 1 (NCBI accession number: NP_000598), Alpha-2-antiplasmin (NCBI accession number: P08697), Complement component C8 alpha chain (NCBI accession number: NP_000553), Complement component C9 (NCBI accession number: NP_001728), Serum amyloid A-1 protein (NCBI accession number: NP_954630), Complement factor D (NCBI accession number: NP_001304264), Serum amyloid A-2 protein (NCBI accession number: NP_110381), Ig-like domain-containing protein (NCBI accession number: Q8N6C5), Complement C1s-A subcomponent (NCBI accession number: P09871), Complement C1s-A subcomponent (NCBI accession number: P09871), N-acetylmuramoyl-L-alanine amidase (NCBI accession number: NP_443122), Carboxypeptidase N catalytic chain (NCBI accession number: NP_001299), Complement C2 (NCBI accession number: P06681), Complement component 7 (NCBI accession number: NP_000578), Mannan-binding lectin serine protease 2 (NCBI accession number: NP_006601), Ficolin-1 (NCBI accession number: NP_001994), Vitamin K-dependent protein S (NCBI accession number: NP_001301006), Mannan-binding lectin serine protease 1 (NCBI accession number: NP 001870), Glyceraldehyde-3-phosphate dehydrogenase (NCBI accession number: P04406), Vitamin K-dependent protein C (NCBI accession number: NP_001362536), Interleukin-1 receptor accessory protein (NCBI accession number: Q9NPH3), Fibronectin (NCBI accession number: P02751), Apolipoprotein B-100 (NCBI accession number: NP 000375), Complement factor H (NCBI accession number: AAI42700), Haptoglobin (NCBI accession number: AAA88080), Immunoglobulin heavy constant mu (NCBI accession number: P01871), Complement component C8 beta chain (NCBI accession number: NP_000057), Protein AMBP (NCBI accession number: NP_001624), Complement component C8 gamma chain (NCBI accession number: NP_000597), Alpha-1-acid glycoprotein 2 (NCBI accession number: NP_000599), Immunoglobulin kappa constant (NCBI accession number: P01834), Mannose-binding protein C (NCBI accession number: NP_001365302), Beta-2-microglobulin (NCBI accession number: NP_004039), Serum amyloid P-component (NCBI accession number: NP_001630), Complement C1s-B (NCBI accession number: P09871), Transthyretin (NCBI accession number: NP_000362), Inter alpha-trypsin inhibitor, heavy chain 4 (NCBI accession number: P19823), Inter-alpha-trypsin inhibitor heavy chain H2 (NCBI accession number: NP_002207), Histidine-rich glycoprotein (NCBI accession number: P04196), Afamin (NCBI accession number: NP_001124), Apolipoprotein A-II (NCBI accession number: NP_001634), Corticosteroid-binding globulin (NCBI accession number: NP_001747), Flavin reductase (NADPH) (NCBI accession number: NP_000704), Pregnancy zone protein (NCBI accession number: NP_002855), Beta-2-glycoprotein 1 (NCBI accession number: NP_000033), Ceruloplasmin (NCBI accession number: NP_000087), Serum paraoxonase/arylesterase 1 (NCBI accession number: NP_000437), Glutathione peroxidase 3 (NCBI accession number: NP 002075), Insulin-like growth factor-binding protein complex acid labile subunit (NCBI accession number: NP_001139478), Apolipoprotein C-III (NCBI accession number: NP_000031), Albumin (NCBI accession number: AAA98797), Apolipoprotein A-I (NCBI accession number: NP_001304947), Apolipoprotein A-IV (NCBI accession number: NP_000473), Apolipoprotein E (NCBI accession number: AAB59397), Complement factor I (NCBI accession number: NP 000195), Hemopexin (NCBI accession number: NP_000604), Plasminogen (NCBI accession number: AAA60113), or Thrombospondin-1 (NCBI accession number: AA136471), a fragment there of, or a variant thereof.

In some examples, the coronal protein-coated nanoparticle disclosed herein comprises proteins specific for target proteins on cancer cells. In some examples, the level of the target protein increase on a cancer cell in comparison to a noncancerous cell. In some examples, the target protein presents on a cancer cell but not on a noncancerous cell. The target proteins on cancer cells can be, for example, hyaluronan, TGF-beta, hyaluronan LDL receptor, fatty acids, vitamin E receptors, kinin receptors, glucocorticoid receptors, riboflavin receptors, LRP1, IL-1, GRP78, lipids, lipid receptors, phospholipids, albumin, Ctrl, ferritin, ferroportin, HDL, selenium (ApoER2+LRP2+LRP1), IGF-1 (IGF-1 receptors), HFE (TfR1), Mannose receptors, and/or fucose receptors. In some examples, the coronal protein-coated nanoparticle disclosed herein comprises proteins specific for those target proteins on cancer cells as shown in Table 5. In some examples, the coronal protein-coated nanoparticle disclosed herein comprises proteins specific for those target proteins on cancer cells as shown in Table 6. In some examples, the coronal protein-coated nanoparticle disclosed herein comprises proteins specific for solid tumor-specific cell proteins.

It should be understood and herein contemplated that the one or more proteins can be directly attached to the HBPE nanoparticle (e.g., through an ionic or covalent bond) or indirectly attached to the HBPE nanoparticle through a sequence that is attached directly to the HBPE nanoparticle. Accordingly, nanoparticle that comprises one or more sequences (e.g., polypeptide sequences) that are specific for the proteins disclosed herein (e.g., the proteins in Tables 4, 5, and/or 6). In some embodiments, the polypeptide sequences can be an antigen-binding fragment. The terms “antigen binding site”, “binding site” and “binding domain” refer to the specific elements, parts or amino acid residues of a polypeptide that bind the proteins disclosed herein.

In some examples, the nanoparticles can further comprise a functionalizing group that can be used to attach targeting ligands, therapeutics, or imaging agents. Examples of suitable functionalizing groups that can be present on the disclosed nanoparticles are azides, amines, alcohols, esters, and the like. In some examples, the nanoparticles disclosed herein are conjugated with one or more targeting ligands. In some examples, the targeting ligand is a folate compound. In some examples, the targeting ligand is a glutamate compound. In some examples, the targeting ligand is a polyglutamated folate compound. In some examples, the targeting ligand is glutamate azido urea. In some examples, the targeting ligand is folate azido urea. In some examples, the targeting ligand is glutamate azido urea. In some examples, the targeting ligand is a bifunctional glutamate-folate hybridized compound. In some examples, the targeting ligand is at high density. In some examples, the targeting ligand is at low density. In some examples, the targeting ligand is at high valency. In some examples, the targeting ligand is at low valency.

In some examples, the nanoparticles comprise one or more anti-cancer therapeutic agents that are encapsulated in the hydrophobic interior of the nanoparticle. In some examples, the one or more therapeutic agents are CT20 peptides. In an aspect, the one or more therapeutic agents are CT20p. In another aspect, the one or more therapeutic agents are mutant CT20 peptides. A CT20 peptide is a C-terminal Bax peptide. Bax is a 21 kD protein of 192 amino acids, comprised of nine alpha helices (Suzuki et al., 2000). Under non-apoptotic conditions, Bax predominantly resides in the cytosol, with a small percentage of the protein localized to the mitochondria (Boohaker et al., 2011; Kaufmann et al., 2003; Putcha et al., 1999). Bax peptides, Bax proteins, and Bax genes are known to those skilled in the art. A disclosed CT20 peptide can comprise SEQ ID NO: 1, SEQ ID NO: 2, SEQ ID NO: 3, SEQ ID NO: 4, SEQ ID NO: 5, and/or SEQ ID NO: 6, or a combination of two or more of SEQ ID NOs: 1-6. For example, in some examples, a disclosed CT20 peptide can be VTIFVAGVLTASLTIWKKMG (SEQ ID NO: 1). In some examples, a disclosed CT20 peptide can be ASLTIWKKMG (SEQ ID NO: 2). In some examples, a disclosed CT20 peptide can be VTIFVAGVLT (SEQ ID NO: 3). In some examples, a disclosed CT20 peptide can be VTIFVAG (SEQ ID NO: 4). In some examples, a disclosed CT20 peptide can be IFVAG (SEQ ID NO: 5). In some examples, a disclosed CT20 peptide can be IWKKMG (SEQ ID NO: 6). In some examples, a disclosed therapeutic composition can comprise one or more CT20 peptides, wherein the one or more CT20 peptides can comprise SEQ ID NO:1, SEQ NO: 2, SEQ ID NO: 3, SEQ ID NO: 4, SEQ ID NO: 5, or SEQ ID NO: 6, or a combination thereof. In some examples, the CT20 peptides are those described in U.S. Pat. Nos. 10,973,925 and 11,129,868, which are incorporated herein by reference in their entireties.

In some examples, the one or more therapeutic agents are anti-metastatic agents. In some examples, the one or more therapeutic agents are anti-androgenic agents. In some examples, the one or more therapeutic agents are anti-neoplastic agents.

Examples of anti-cancer drugs or anti-neoplastic drugs include, but are not limited to, the following: Acivicin; Aclarubicin; Acodazole Hydrochloride; AcrQnine; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflomithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Gold Au 198; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safmgol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride.

Other anti-neoplastic compounds include: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; atrsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docosanol; dolasetron; doxifluridine; droloxifene; dronabinol; duocannycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; fmasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A +myobacterium cell wall sk; mopidamol; multiple drug resistance genie inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone +pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfmosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer.

In some embodiments, the anti-cancer therapeutic agent comprises paclitaxel, docetaxel, or cabazitaxel.

The anti-cancer therapeutic agent can be hydrophobic and encapsulated in the interior of the nanoparticle.

In some embodiments, the nanoparticle further comprises a chelating ligand (e.g., desferrioxamine (DFO).

In some embodiments, the nanoparticle further comprises polyethylene glycol (PEG).

As used herein, radiosensitizers make a cancer cell more likely to be damaged. Radiosensitizers enhance the sensitivity of cancer cells and/or a tumor to ionizing radiation, thereby increasing the efficacy of radiotherapy. Examples of radiosensitizers include gemcitabine, 5-fluorouracil, pentoxifylline, and vinorelbine.

The majority of chemotherapeutic drugs can be divided in to: alkylating agents (e.g., cisplatin, carboplatin, oxaliplatin, mechlorethamine, cyclophosphamide, chlorambucil), anti-metabolites (e.g., azathioprine, mercaptopurine), anthracyclines, plant alkaloids and terpenoids (e.g., vinca alkaloids (e.g., vincristine, vinblastine, vinorelbine, vindesine, and podophyllotoxin) and taxanes (e.g., paclitaxel and docetaxel), topoisomerase inhibitors (e.g., irinotecan, topotecan, amsacrine, etoposide, etoposide phosphate, and teniposide), monoclonal antibodies (e.g., trastuzumab, cetuximab, rituximab, bevacizumab), other antitumour agents (e.g., dactinomycin), and hormonal therapy (e.g., steroids such as dexamethasone, finasteride, aromatase inhibitors, and gonadotropin-releasing hormone agonists).

In an aspect, the nanoparticles comprise an imaging compound. In aspect, the imaging compound is a PET detectable compound. In an aspect, the PET detectable compound is ⁸⁹Zr. In an aspect, the PET detectable compound is CU or other PET detectable compounds.

In some embodiments, the nanoparticle has a diameter from about 1 nm to about 1000 nm. In some embodiments, the nanoparticle has a diameter less than, for example, about 1000 nm, about 950 nm, about 900 nm, about 850 nm, about 800 nm, about 750 nm, about 700 nm, about 650 nm, about 600 nm, about 550 nm, about 500 nm, about 450 nm, about 400 nm, about 350 nm, about 300 nm, about 290 nm, about 280 nm, about 270 nm, about 260 nm, about 250 nm, about 240 nm, about 230 nm, about 220 nm, about 210 nm, about 200 nm, about 190 nm, about 180 nm, about 170 nm, about 160 nm, about 150 nm, about 140 nm, about 130 nm, about 120 nm, about 110 nm, about 100 nm, about 90 nm, about 80 nm, about 70 nm, about 60 nm, about 50 nm, about 40 nm, about 30 nm, about 20 nm, or about 10 nm. In some embodiments, the nanoparticle has a diameter, for example, from about 20 nm to about 1000 nm, from about 20 nm to about 800 nm, from about 20 nm to about 700 nm, from about 30 nm to about 600 nm, from about 30 nm to about 500 nm, from about 40 nm to about 400 nm, from about 40 nm to about 300 nm, from about 40 nm to about 250 nm, from about 50 nm to about 250 nm, from about 50 nm to about 200 nm, from about 50 nm to about 150 nm, from about 60 nm to about 150 nm, from about 70 nm to about 150 nm, from about 80 nm to about 150 nm, from about 90 nm to about 150 nm, from about 100 nm to about 150 nm, from about 110 nm to about 150 nm, from about 120 nm to about 150 nm, from about 90 nm to about 140 nm, from about 90 nm to about 130 nm, from about 90 nm to about 120 nm, from 100 nm to about 140 nm, from about 100 nm to about 130 nm, from about 100 nm to about 120 nm, from about 100 nm to about 110 nm, from about 110 nm to about 120 nm, from about 110 nm to about 130 nm, from about 110 nm to about 140 nm, from about 90 nm to about 200 nm, from about 100 nm to about 195 nm, from about 110 nm to about 190 nm, from about 120 nm to about 185 nm, from about 130 nm to about 180 nm, from about 140 nm to about 175 nm, from 150 nm to 175 nm, or from about 150 nm to about 170 nm. In some embodiments, the nanoparticle has a diameter from about 100 nm to about 250 nm. In some embodiments, the nanoparticle has a diameter from about 150 nm to about 175 nm. In some embodiments, the nanoparticle has a diameter from about 135 nm to about 175 nm. The particles can have any shape but are generally spherical in shape.

A nanoparticle has a surface charge that attracts ions having opposite charge to the nanoparticle surface. Such a double layer of ions travels with the nanoparticle. Zeta potential refers to the electrostatic potential at the electrical double layer. In some embodiments, the nanoparticle disclosed herein has a zeta potential ranging from about −10 mV to about −100 mV, about −20 mV to about −100 mV, about −30 mV to about −100 mV, about −40 mV to about −100 mV, about −50 mV to about −100 mV, about −60 mV to about −100 mV, about −10 mV to about −80 mV, about −20 mV to about −70 mV, about −30 mV to about −60 mV, less than about −5 mV, less than about −6 mV, less than about −7 mV, less than about −9 mV, less than about −10 mV, less than about −11 mV, less than about −12 mV, less than about 13 mV, less than about −14 mV, less than about −15 mV, less than about −16 mV, less than about −17 mV, less than about −18 mV, less than about −19 mV, less than about −20 mV, less than about −21 mV, less than about −22 mV, less than about −23 mV, less than about −24 mV, less than about −25 mV, less than about −26 mV, less than about −27 mV, less than about −28 mV, less than −29 mV, less than −30 mV, less than −31 mV, or less than −32 mV. In some embodiments, the coronal protein-coated nanoparticle disclosed herein has a zeta potential about −10 mV, about −12 mV, about −13 mV, about −14 mV, about −15 mV, about −16 mV, about −17 mV, about −18 mV, about −20 mV, about −22 mV, about −24 mV, about −26 mV, about −28 mV, about −30 mV, about −40 mV, about −41 mV, about −42 mV, about −43 mV, about −44 mV, about −45 mV, about −46 mV, about −47 mV, about −48 mV, about −49 mV, about −50 mV, about −55 mV, about −60 mV, about −70 mV, about −80 mV, about −90 mV, or about −100 mV.

The molecular weight (MW) of the nanoparticle disclosed herein can be from about 1,000 Da to about 100,000 Da. For example, the nanoparticle can have a MW of from about 1,000 Da to about 75,000 Da, from about 1,000 Da to about 50,000 Da, from about 1,000 Da to about 25,000 Da, from about 10,000 Da to about 100,000 Da, from about 10,000 Da to about 75,000 Da, from about 10,000 Da to about 50,000 Da, from about 25,000 Da to about 100,000 Da, from about 25,000 Da to about 75,000 Da, from about 50,000 Da to about 100,000 Da, or from about 50,000 Da to about 75,000 Da.

The coronal protein-coated nanoparticles disclosed herein can increase uptake of the nanoparticles by a cancer cell (e.g., by at least about 10%, at least about 20%, or at least about 30%, or at least about 40%, or at least about 50%, or at least about 60%, or at least about 70%, or at least about 80%, or at least about 90%, or at least about a 2-fold, or at least about a 3-fold, or at least about a 4-fold, or at least about a 5-fold or at least about a 10-fold increase) in comparison to nanoparticles that are not coated with the coronal proteins disclosed herein.

The coronal protein-coated nanoparticles disclosed herein can reduce uptake of the nanoparticles by a noncancerous cell (e.g., by at least about 10%, at least about 20%, or at least about 30%, or at least about 40%, or at least about 50%, or at least about 60%, or at least about 70%, or at least about 80%, or at least about 90%, or at least about a 2-fold, or at least about a 3-fold, or at least about a 4-fold, or at least about a 5-fold or at least about a 10-fold decrease) in comparison to nanoparticles that are not coated with the coronal proteins disclosed herein.

Accordingly, the amounts of anti-cancer therapeutic agent dispersed or encapsulated in the nanoparticle composition disclosed herein can be generally smaller, e.g., at least about 10% smaller, than the amount of anti-cancer therapeutic agent present in the current dosage of the treatment regimen (i.e., without nanoparticle composition) required for producing essentially the same therapeutic effect. Indeed, anti-cancer therapeutic agent encapsulated in, or adhered to, a nanoparticle composition can potentially increase duration of the therapeutic effect for anti-cancer therapeutic agent. Stated another way, encapsulating anti-cancer therapeutic agent in a nanoparticle composition or adhering anti-cancer therapeutic agent to the nanoparticle composition can increase its therapeutic efficacy, i.e., a smaller amount of anti-cancer therapeutic agent encapsulated in a nanoparticle, as compared to the amount present in a typical one dosage administered for cancer treatment, can achieve essentially the same therapeutic effect. Accordingly, the nanoparticle composition can comprise anti-cancer therapeutic agent in an amount which is less than the amount traditionally recommended for one dosage of anti-cancer therapeutic agent, while achieving essentially the same therapeutic effect. For example, if the traditionally recommended dosage of an anti-cancer therapeutic agent is X amount, then the nanoparticle composition can comprise anti-cancer therapeutic agent in an amount of about 0.9×, about 0.8×, about 0.7×, about 0.6×, about 0.5×, about 0.4×, about 0.3×, about 0.2×, about 0.1× or less. Without wishing to be bound by the theory, this can allow administering a lower dosage of anti-cancer therapeutic agent in a nanoparticle to obtain a therapeutic effect which is similar to when a higher dosage is administered without the nanoparticle composition. Low-dosage administration of anti-cancer therapeutic agent can reduce side effects of the anti-cancer therapeutic agent, if any, and/or reduce likelihood of the subject's resistance to anti-cancer therapeutic agent after administration for a period of time.

In some aspects, disclosed herein is a therapeutic composition comprising the coronal protein-coated nanoparticle disclosed herein.

Also disclosed herein is a cancer therapeutic composition comprising the coronal protein-coated nanoparticle disclosed herein.

In an aspect, a disclosed therapeutic composition can be administered to a subject repeatedly. In some examples, a disclosed therapeutic composition can be administered to the subject at least two times. In some examples, a disclosed therapeutic composition can be administered to the subject two or more times. In some examples, a disclosed therapeutic composition can be administered at routine or regular intervals. For example, in some examples, a disclosed therapeutic composition can be administered to the subject one time per day, or two times per day, or three or more times per day. In some examples, a disclosed therapeutic composition can be administered to the subject daily, or one time per week, or two times per week, or three or more times per week, etc. In some examples, a disclosed therapeutic composition can be administered to the subject weekly, or every other week, or every third week, or every fourth week, etc. In some examples, a disclosed therapeutic composition can be administered to the subject monthly, or every other month, or every third month, or every fourth month, etc. In some examples, the repeated administration of a disclosed composition occurs over a pre-determined or definite duration of time. In some examples, the repeated administration of a disclosed composition occurs over an indefinite period of time.

In some examples, the disclosed subject matter relates to pharmaceutical compositions comprising a disclosed composition comprising the coronal protein-coated nanoparticles disclosed herein. In some examples, the disclosed composition further comprises an imaging compound and one or more therapeutic agents encapsulated in the hydrophobic interior of the nanoparticle. In some examples, the disclosed subject matter relates to pharmaceutical compositions comprising a disclosed cancer therapeutic composition comprising the disclosed composition. In some examples, a pharmaceutical composition can be provided comprising a therapeutically effective amount of at least one disclosed composition and a pharmaceutically acceptable carrier.

### Methods of Treating Cancer

Disclosed herein are methods of treating cancer in a subject in need thereof, comprising administering to the subject a therapeutically effective amount of the coronal protein-coated nanoparticle disclosed herein. In some embodiments, the coronal protein-coated nanoparticle comprises one or more proteins selected from of the group consisting of Albumin, Complement C3, Pregnancy zone protein, Apolipoprotein A-I, Cluster of GLOBIN domain-containing protein, GLOBIN domain-containing protein, Serotransferrin, Ceruloplasmin, Cluster of Murinoglobulin-1, Murinoglobulin-1, Hemopexin, Cluster of Serine protease inhibitor A3K, Serine protease inhibitor A3K, Kininogen-1, Hemoglobin subunit beta-2, Plasminogen, Haptoglobin, Cluster of Alpha-1-antitrypsin 1-4, Alpha-2-HS-glycoprotein, Thrombospondin-1, Hemoglobin subunit alpha, Alpha-1-antitrypsin 1-4, Murinoglobulin-2, Alpha-1-antitrypsin 1-1, Alpha-1-antitrypsin 1-2, Inter alpha-trypsin inhibitor, heavy chain 4, Fibronectin, Vitamin D-binding protein, Prothrombin, Spectrin beta chain, erythrocytic, Serine protease inhibitor A3M, Cluster of Complement factor H, Complement factor H, Apolipoprotein E, Apolipoprotein A-IV, Cluster of Carboxylesterase 1C, Complement factor B, Beta-2-glycoprotein 1, Cluster of Ankyrin-1, Ankyrin-1, Spectrin alpha chain, erythrocytic 1, Carboxylesterase 1C, Apolipoprotein B-100, Serine protease inhibitor A3N, Inhibitor of carbonic anhydrase, Complement factor I, Complement C4-B, Histidine-rich glycoprotein, Cluster of Actin, cytoplasmic 1, Actin, cytoplasmic 1, Afamin, Inter-alpha-trypsin inhibitor heavy chain H2, Protein AMBP, Actin, alpha cardiac muscle 1, Inter-alpha-trypsin inhibitor heavy chain H3, LRRCT domain-containing protein, Vitronectin, Clusterin, Phosphatidylinositol-glycan-specific phospholipase D, Plasma kallikrein, Complement C5, Cluster of Keratin, type II cytoskeletal 2 epidermal, Cluster of H-2 class I histocompatibility antigen, Q10 alpha chain, H-2 class I histocompatibility antigen, Q10 alpha chain, Band 3 anion transport protein, Carboxylic ester hydrolase, Immunoglobulin heavy constant mu, Inter-alpha-trypsin inhibitor heavy chain H1, Serum amyloid P-component, Apolipoprotein M, Beta-actin-like protein 2, Gelsolin, Antithrombin-III, Alpha-1B-glycoprotein, CD5 antigen-like, Sulfhydryl oxidase 1, HMW kininogen-II, Ig-like domain-containing protein, Fetuin-B, Properdin, Ig gamma-2B chain C region, Complement component C8 alpha chain, Cluster of Heat shock cognate 71 kDa protein, Serum amyloid A-1 protein, Immunoglobulin kappa constant, Glutathione peroxidase 3, Heat shock cognate 71 kDa protein, Alpha-1-antitrypsin 1-5, Apolipoprotein A-II, Talin-1, Coagulation factor V, Carboxypeptidase N subunit 2, Fibrinogen alpha chain, Ig-like domain-containing protein, Coagulation factor X, Immunoglobulin heavy constant gamma 3, Carbonic anhydrase 1, Keratin, type II cytoskeletal 5, Apolipoprotein D, Ig gamma-1 chain C region, membrane-bound form, Apolipoprotein C-IV, Alpha-2-antiplasmin, Beta-2-microglobulin, Immunoglobulin heavy constant gamma 2C, Alpha-1-acid glycoprotein 1, Complement component C8 beta chain, N-acetylmuramoyl-L-alanine amidase, Vitamin K-dependent protein Z, Cluster of Filamin-A, Filamin-A, BPI fold-containing family A member 2, Serum paraoxonase/arylesterase 1, Serum amyloid A-4 protein, Epidermal growth factor receptor, Zinc-alpha-2-glycoprotein, Phosphatidylcholine-sterol acyltransferase, Mannose-binding protein C, Carboxypeptidase N catalytic chain, SCY domain-containing protein, Interleukin-1 receptor accessory protein, Immunoglobulin J chain, Complement C1q subcomponent subunit A, Keratin, type II cytoskeletal 2 epidermal, Keratin, type II cytoskeletal 1, IF rod domain-containing protein, Keratin, type II cytoskeletal 1b, Keratin, type II cytoskeletal 75, Carboxylic ester hydrolase, Fibrinogen gamma chain, Cluster of Keratin, type I cytoskeletal 10, Keratin, type I cytoskeletal 10, Serum amyloid A-2 protein, Transthyretin, Carbonic anhydrase 2, Immunoglobulin heavy constant alpha, Plasma protease C1 inhibitor, Transitional endoplasmic reticulum ATPase, Coagulation factor XII, Glyceraldehyde-3-phosphate dehydrogenase, Mannan-binding lectin serine protease 2, Alpha-1-acid glycoprotein 2, sp|Q8BXA7|PHLP2_DECOY, Keratin, type II cytoskeletal 73, IF rod domain-containing protein, Keratin, type II cytoskeletal 2 oral, Apolipoprotein C-III, Predicted gene 4788, Endoplasmic reticulum chaperone BiP, IF rod domain-containing protein, Selenoprotein P, Platelet-activating factor acetylhydrolase, Complement component C9, sp|P46656|ADX_DECOY, Corticosteroid-binding globulin, Platelet factor 4, Cluster of Alpha-amylase 1, Alpha-amylase 1, Heparin cofactor 2, Ig-like domain-containing protein, Cluster of Tubulin alpha-4A chain, Tubulin alpha-4A chain, Complement factor D, Retinol-binding protein 4, 14-3-3 protein zeta/delta, Secreted phosphoprotein 24, Macrophage colony-stimulating factor 1 receptor, Lumican, 55 kDa erythrocyte membrane protein, sp|Q9WTU0|PHF2 DECOY, sp|P59242|CING_DECOY, C-reactive protein, Coagulation factor IX, Peroxiredoxin-2, Vitamin K-dependent protein S, sp|Q571H0|NPA1P_DECOY, Ficolin-1, Ig lambda-2 chain C region, Profilin-1, Extracellular superoxide dismutase [Cu—Zn], Complement C1q subcomponent subunit C, Biotinidase, Keratin, type II cytoskeletal 79, Keratin, type II cytoskeletal 8, Keratin, type II cytoskeletal 74, Ankyrin-3, Ankyrin-2, C4b-binding protein, Apolipoprotein C-I, Keratin, type I cytoskeletal 16, Pigment epithelium-derived factor, Endogenous retroviral sequence 3, Predicted gene 382, Cluster of Myosin-9, Myosin-9, Myosin-11, sp|P98083|SHC1_DECOY, Hepatocyte growth factor activator, Pancreatic alpha-amylase, Aamy domain-containing protein, Ig-like domain-containing protein, Keratin, type I cytoskeletal 28, Spectrin alpha chain, non-erythrocytic 1, Extracellular matrix protein 1, Fibrinogen beta chain, Mannose-binding protein A, Carboxypeptidase B2, Tubulin alpha-1B chain, Complement component C8 gamma chain, Keratin, type I cytoskeletal 42, Protein PRRC2B, Lysosomal alpha-mann, OSidase, sp|Q6RUT8|CC154_DECOY, sp|Q8BMD6|TM260_DECOY, Ras-related protein Rap-1b, Alpha-actinin-1, Proteoglycan 4, Ig-like domain-containing protein, von Willebrand factor, Serglycin, sp|A2AAY5|SPD2B_DECOY, sp|Q9JLV2|TP4AP_DECOY, Hyaluronan-binding protein 2, Disks large homolog 5, sp|Q7TSJ2|MAP6_DECOY, Amyloid-beta A4 precursor protein-binding family B member 2, sp|Q9Z1P8|ANGL4_DECOY, tr|J3QNP2|J3QNP2_DECOY, Insulin-like growth factor-binding protein complex acid labile subunit, Fermitin family homolog 3, Ig-like domain-containing protein, Ig kappa chain V-II region 26-10, Ig kappa chain V-V region MOPC 41, L-selectin, Ig kappa chain V-VI region XRPC 44, Leukemia inhibitory factor receptor, Platelet glycoprotein Ib alpha chain, Hepatocyte growth factor-like protein, Nuclear receptor subfamily 1 group D member 2, sp|B2RQE8|RHG42_DECOY, Lysozyme C-2, Tubulin alpha-8 chain, Vitamin K-dependent protein C, RIKEN cDNA 9530053A07 gene, Ig-like domain-containing protein, Ig-like domain-containing protein, Superoxide dismutase [Cu—Zn], Triosephosphate isomerase, Anaphylatoxin-like domain-containing protein, Hepcidin, Peptidyl-prolyl cis-trans isomerase A, Metalloproteinase inhibitor 3, Ig-like domain-containing protein, Immunoglobulin heavy variable 1-15, Progranulin, Testisin, tr|Q91X36|Q91X36_DECOY, sp|Q8VEE4|RFA1_DECOY, Complement C1r-A subcomponent, Carboxypeptidase Q, Dematin, Ig heavy chain V region MOPC 47A, T-lymphoma invasion and metastasis-inducing protein 1, Ras-related protein Rab-19, Protein disulfide-isomerase A3, Proto-oncogene tyrosine-protein kinase receptor Ret, Ig-like domain-containing protein, Monocarboxylate transporter 1, sp|P67984|RL22_DECOY, Phosphatidylinositide phosphatase SAC2, Complement factor H-related 2, Serine protease inhibitor A3G, Neurofilament heavy polypeptide, H-2 class I histocompatibility antigen, alpha chain, H-2 class I histocompatibility antigen, Q8 alpha chain, Ig-like domain-containing protein, Filamin-B, Filamin-C, sp|A2ASS6|TITIN_DECOY, phospholipid transfer protein, sp|Q6ZWQ0|SYNE2_DECOY, Desmoplakin, Pericentrin, sp|Q80SU7|GVIN1 DECOY, Complement component 6, Transmembrane protein KIAA1109, sp|Q3UHF7|ZEP2_DECOY, sp|A2BH40|ARI1A_DECOY, E3 ubiquitin-protein ligase MIB1, tr|B1AR51|B1AR51_DECOY, Cluster of Tubulin beta-4B chain, Tubulin beta-4B chain, Tubulin beta-3 chain, Tubulin beta-2A chain, sp|Q8R3B7|BRD8_DECOY, sp|Q6A078|CE290_DECOY, sp|Q66JQ7|KNL1_DECOY, sp|A2ARZ3|FSIP2_DECOY, Myosin light polypeptide 6, Cathelicidin antimicrobial peptide, Tubulin beta-1 chain, Keratin, type I cytoskeletal 13, sp|Q9R269|PEPL_DECOY, PDZ domain-containing protein, sp|Q61285|ABCD2 DECOY, RIKEN cDNA 4930407110 gene, Cluster of sp|A2A432|CUL4B_DECOY, sp|A2A432|CUL4B_DECOY, sp|Q3TCH7|CUL4A_DECOY, Cystatin domain-containing protein, VWFA domain-containing protein, sp|Q60988|STIL_DECOY, sp|Q8WTY4|CPIN1_DECOY, Klotho, sp|Q9R1L5|MAST1_DECOY, Vasculin-like protein 1, Cluster of Ig kappa chain V-V region HP R16.7, Ig kappa chain V-V region HP R16.7, Ig kappa chain V-V region HP 123E6, Pyruvate kinase PKM, Bridging integrator 2, Angiotensinogen, Peptidase Si domain-containing protein, Apolipoprotein N, tr|B2RPU8|B2RPU8_DECOY, Histone-lysine N-methyltransferase 2D, DNA topoisomerase 3-alpha, Cysteine and glycine-rich protein 1, Vascular cell adhesion protein 1, Apolipoprotein A-V, Flavin reductase (NADPH), Dual specificity protein phosphatase 3, Peptidase Si domain-containing protein, Ras-related protein Rab-8B, Nucleolar and coiled-body phosphoprotein 1, sp|B2RPV6|MMRN1_DECOY, Proprotein convertase subtilisin/kexin type 9, sp|P97350|PKP1_DECOY, Isoleucine-tRNA ligase, mitochondrial, sp|Q7TQC5|APTX_DECOY, sp|O88329|MYO1A_DECOY, sp|Q9D920|BORC5_DECOY, Cilia- and flagella-associated protein 221, Ig-like domain-containing protein, Ig kappa chain V-III region PC 3741/TEPC 111, Ig-like domain-containing protein, Ig-like domain-containing protein, Ig-like domain-containing protein, Ig-like domain-containing protein, Ig-like domain-containing protein, Angiopoietin-1, Cofilin-1, EH domain-containing protein 4, Ras-related C3 botulinum toxin substrate 1, Basigin, SPARC-like protein 1, sp|Q8K370|ACD10_DECOY, sp|Q99N80|SYTL1_DECOY, Mannan-binding lectin serine protease 1, BPI fold-containing family A member 1, Cardiotrophin-like cytokine factor 1, A disintegrin and metallopeptidase domain 6B, Polypeptide N-acetylgalactosaminyltransferase 13, Coagulation factor XIII B chain, Antileukoproteinase, tr|E9Q6R1|E9Q6R1 DECOY, Proteasome subunit alpha type-2, sp|Q8CFS6|KCNV2_DECOY, sp|O08550|KMT2B_DECOY, and Discoidin domain-containing receptor 2, a fragment thereof, or a variant thereof.

In some embodiments, the coronal protein-coated nanoparticle comprises one or more proteins selected from of the group consisting of complement C3, alp ha-2-HS-glycoprotein, complement factor B, vitronectin, clusterin, inhibitor of carbonic anhydrase, H-2 class I histocompatibility antigen, Q10 alpha chain, complement C5, carboxypeptidase N subunit 2, plasma protease C1 inhibitor, alpha-1-acid glycoprotein 1, alpha-2-antiplasmin, complement component C8 alpha chain, complement component C9, serum amyloid A-1 protein, complement factor D, serum amyloid A-2 protein, Ig-like domain-containing protein, complement C1s-A subcomponent, N-acetylmuramoyl-L-alanine amidase, carboxypeptidase N catalytic chain, complement C2, complement component 7, mannan-binding lectin serine protease 2, ficolin-1, complement C1r-A subcomponent, vitamin K-dependent protein S, mannan-binding lectin serine protease 1, glyceraldehyde-3-phosphate dehydrogenase, vitamin K-dependent protein C, interleukin-1 receptor accessory protein, fibronectin, apolipoprotein B-100, complement factor H, haptoglobin, immunoglobulin heavy constant mu, complement component C8 beta chain, Ig gamma-2B chain C region, protein AMBP, Ig gamma-1 chain C region, membrane-bound form, complement component C8 gamma chain, alpha-1-acid glycoprotein 2, immunoglobulin kappa constant, mannose-binding protein C, beta-2-microglobulin, serum amyloid P-component, complement C1s-B subcomponent, transthyretin, inter alpha-trypsin inhibitor, heavy chain 4, Inter-alpha-trypsin inhibitor heavy chain H2, histidine-rich glycoprotein, afamin, apolipoprotein A-II, corticosteroid-binding globulin, flavin reductase (NADPH), pregnancy zone protein, beta-2-glycoprotein 1, ceruloplasmin, serum paraoxonase/arylesterase 1, glutathione peroxidase 3, insulin-like growth factor-binding protein complex acid labile subunit, apolipoprotein C-III, albumin, apolipoprotein A-I, apolipoprotein A-IV, apolipoprotein E, complement factor I, hemopexin, plasminogen, and thrombospondin-1, or a fragment thereof.

In some examples, the one or more proteins are selected from the group consisting of Inter alpha-trypsin inhibitor, heavy chain 4, alpha-2-HS-glycoprotein, inter-alpha-trypsin inhibitor heavy chain H2, clusterin, histidine-rich glycoprotein, afamin, carboxypeptidase N subunit 2, apolipoprotein A-II, corticosteroid-binding globulin, and flavin reductase (NADPH), or a fragment thereof.

In some examples, the one or more proteins are selected from the group consisting of pregnancy zone protein, apolipoprotein B-100, beta-2-glycoprotein 1, ceruloplasmin, serum paraoxonase/arylesterase 1, glutathione peroxidase 3, insulin-like growth factor-binding protein complex acid labile subunit, apolipoprotein C-III, beta-2-microglobulin, and mannose-binding protein C, or a fragment thereof.

In some examples, the one or more proteins are selected from the group consisting of albumin, alpha-2-HS-glyco protein, apolipoprotein A-I, apolipoprotein A-IV, apolipoprotein B-100, apolipoprotein E, beta-2-glycoprotein 1, ceruloplasmin, clusterin, complement factor I, hemopexin, histidine-rich glycoprotein, inter alpha-trypsin inhibitor, heavy chain 4, plasminogen, pregnancy zone protein, and thrombospondin-1, or a fragment thereof.

In some examples, the coronal protein-coated nanoparticle disclosed herein comprises thrombospondin-1 or a fragment thereof.

In some examples, the nanoparticle further comprises an imaging compound. In an aspect, the nanoparticle has one or more therapeutic agents encapsulated in the hydrophobic interior of the nanoparticle. Additional therapeutical and/or radiolabeled compounds can be administered with (either separately, before and/or after, or simultaneously) with the nanoparticles.

In some examples, the one or more therapeutic agents are or one or more anti-cancer therapeutic agents. In an aspect, a disclosed therapeutic composition can comprise one or more anti-cancer therapeutic agents. In some examples, the one or more anti-cancer therapeutic agents can comprise cisplatin. In some examples, the one or more anti-cancer therapeutic agents induce apoptosis. In some examples, a disclosed therapeutic composition can comprise one or more chemotherapeutic drugs. In some examples, a disclosed therapeutic composition can comprise one or more radiosensitizers. In some examples, a disclosed therapeutic composition can comprise a pharmaceutically acceptable carrier.

In some examples, the nanoparticles comprise one or more anti-cancer therapeutic agents that are encapsulated in the hydrophobic interior of the nanoparticle. In some examples, the one or more therapeutic agents are CT20 peptides. In an aspect, the one or more therapeutic agents are CT20p. In another aspect, the one or more therapeutic agents are mutant CT20 peptides. A disclosed CT20 peptide can comprise SEQ ID NO: 1, SEQ ID NO: 2, SEQ ID NO: 3, SEQ ID NO: 4, SEQ ID NO: 5, and/or SEQ ID NO: 6, or a combination of two or more of SEQ ID NOs: 1-6. For example, in some examples, a disclosed CT20 peptide can be VTIFVAGVLTASLTIWKKMG (SEQ ID NO: 1). In some examples, a disclosed CT20 peptide can be ASLTIWKKMG (SEQ ID NO: 2). In some examples, a disclosed CT20 peptide can be VTIFVAGVLT (SEQ ID NO: 3). In some examples, a disclosed CT20 peptide can be VTIFVAG (SEQ ID NO: 4). In some examples, a disclosed CT20 peptide can be IFVAG (SEQ ID NO: 5). In some examples, a disclosed CT20 peptide can be IWKKMG (SEQ ID NO: 6). In some examples, a disclosed therapeutic composition can comprise one or more CT20 peptides, wherein the one or more CT20 peptides can comprise SEQ ID NO:1, SEQ NO: 2, SEQ ID NO: 3, SEQ ID NO: 4, SEQ ID NO: 5, or SEQ ID NO: 6, or a combination thereof. In some examples, the CT20 peptides and the uses thereof are those described in U.S. Pat. Nos. 10,973,925 and 11,129,868, which are incorporated herein by reference in their entireties.

A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer. In some embodiments, the cancer is breast cancer. In some embodiments, the cancer is triple negative breast cancer. In some embodiments, the cancer is lung cancer. In some embodiments, the cancer is prostate cancer. In some embodiments, the cancer is liver cancer.

The coronal protein-coated nanoparticles disclosed herein can increase uptake of the nanoparticles by a cancer cell (e.g., by at least about 10%, at least about 20%, or at least about 30%, or at least about 40%, or at least about 50%, or at least about 60%, or at least about 70%, or at least about 80%, or at least about 90%, or at least about a 2-fold, or at least about a 3-fold, or at least about a 4-fold, or at least about a 5-fold or at least about a 10-fold increase) in comparison to nanoparticles that are not coated with the coronal proteins disclosed herein. The coronal protein-coated nanoparticles disclosed herein can reduce uptake of the nanoparticles by a noncancerous cell (e.g., by at least about 10%, at least about 20%, or at least about 30%, or at least about 40%, or at least about 50%, or at least about 60%, or at least about 70%, or at least about 80%, or at least about 90%, or at least about a 2-fold, or at least about a 3-fold, or at least about a 4-fold, or at least about a 5-fold or at least about a 10-fold decrease) in comparison to nanoparticles that are not coated with the coronal proteins disclosed herein.

Accordingly, the amounts of anti-cancer therapeutic agent dispersed or encapsulated in the nanoparticle composition disclosed herein can be generally smaller, e.g., at least about 10% smaller, than the amount of anti-cancer therapeutic agent present in the current dosage of the treatment regimen (i.e., without nanoparticle composition) required for producing essentially the same therapeutic effect. Indeed, anti-cancer therapeutic agent encapsulated in, or adhered to, a nanoparticle composition can potentially increase duration of the therapeutic effect for anti-cancer therapeutic agent. Stated another way, encapsulating anti-cancer therapeutic agent in a nanoparticle composition or adhering anti-cancer therapeutic agent to the nanoparticle composition can increase its therapeutic efficacy, i.e., a smaller amount of anti-cancer therapeutic agent encapsulated in a nanoparticle, as compared to the amount present in a typical one dosage administered for cancer treatment, can achieve essentially the same therapeutic effect. Accordingly, the nanoparticle composition can comprise anti-cancer therapeutic agent in an amount which is less than the amount traditionally recommended for one dosage of anti-cancer therapeutic agent, while achieving essentially the same therapeutic effect. For example, if the traditionally recommended dosage of an anti-cancer therapeutic agent is X mg amount, then the nanoparticle composition can comprise anti-cancer therapeutic agent in an amount of about 0.9×, about 0.8×, about 0.7×, about 0.6×, about 0.5×, about 0.4×, about 0.3×, about 0.2×, about 0.1× or less. Without wishing to be bound by the theory, this can allow administering a lower dosage of anti-cancer therapeutic agent in a nanoparticle to obtain a therapeutic effect which is similar to when a higher dosage is administered without the nanoparticle composition. Low-dosage administration of anti-cancer therapeutic agent can reduce side effects of the anti-cancer therapeutic agent, if any, and/or reduce likelihood of the subject's resistance to anti-cancer therapeutic agent after administration for a period of time.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

Dosages are typically modified according to the characteristics of the subject (weight, gender, age, etc.), severity of disease (e.g., degree of metastasis), specifics and purity of the active agent to be administered, route of administration, nature of the formulation, and numerous other factors. Generally, the active agent (e.g., an anti-cancer therapeutic agent) is administered to the subject at a dosage ranging from 0.01 μg/kg body weight to 100 g/kg body weight. In some embodiments, the active agent is administered to the subject at a dosage of from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. The dosage of administration for the active agent disclosed herein can be from about 0.01 mg/kg body weight to about 100 mg/kg body weight. In some examples, the dosage is about 0.01 mg/kg body weight, about 0.05 mg/kg body weight, about 0.1 mg/kg body weight, about 0.5 mg/kg body weight, about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 8.5 mg/kg body weight, about 9 mg/kg body weight, about 9.5 mg/kg body weight, about 10 mg/kg body weight, about 11 mg/kg body weight, about 12 mg/kg body weight, about 13 mg/kg body weight, about 14 mg/kg body weight, about 15 mg/kg body weight, about 20 mg/kg body weight, about 25 mg/kg body weight, about 30 mg/kg body weight, about 35 mg/kg body weight, about 40 mg/kg body weight, about 45 mg/kg body weight, about 50 mg/kg body weight, about 55 mg/kg body weight, about 60 mg/kg body weight, about 65 mg/kg body weight, about 70 mg/kg body weight, about 75 mg/kg body weight, about 80 mg/kg body weight, about 85 mg/kg body weight, about 90 mg/kg body weight, about 95 mg/kg body weight, or about 100 mg/kg body weight. Dosages above or below the range cited above may be administered to the individual patient if desired.

Dosing frequency for the composition of any preceding aspects, includes, but is not limited to, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### Methods of Making Nanoparticles

In some aspects, disclosed herein is a method of generating one or more coronal protein-coated nanoparticles, comprising contacting one or more nanoparticles with a serum sample obtained from a subject, wherein the serum samples are obtained from a subject infected by influenza A virus, wherein the nanoparticle is a hyperbranched polyester (HBPE) nanoparticle.

In some examples, the serum sample is obtained from the subject on day 3, day 4, day 5, day 6, day 7, day 8, day 9, day 10, day 11, or day 12 post infection of influenza A virus. In some examples, the serum sample is obtained from the subject on days 3-4 post infection of influenza A virus. In some examples, the serum sample is obtained from the subject on days 4-5 post infection of influenza A virus. In some examples, the serum sample is obtained from the subject on days 5-6 post infection of influenza A virus. In some examples, the serum sample is obtained from the subject on days 5-7 post infection of influenza A virus. In some examples, the serum sample is obtained from the subject on days 6-7 post infection of influenza A virus. In some examples, the serum sample is obtained from the subject on days 7-8 post infection of influenza A virus. In some examples, the serum sample is obtained from the subject on days 8-9 post infection of influenza A virus. In some examples, the serum sample is obtained from the subject on day 5 post infection of influenza A virus. In some examples, the serum sample is obtained from the subject on day 6 post infection of influenza A virus. In some examples, the serum sample is obtained from the subject on day 7 post infection of influenza A virus. Sera can be collected from the subject days 3-9 of the infection and have a unique profile of sera proteins from innate and adaptive immune response. Due to the immune cells shedding more proteins into the sera because of the virus infection, a more complex sera can be obtained than is found in a healthy (non-infected) subject.

In some examples, wherein the nanoparticles are in contact with the serum sample at room temperature. In some examples, the nanoparticles are in contact with the serum sample for at least 10 min (e.g., at least 12 min, at least 15 min, at least 20 min, at least 30 min, at least 40 min, at least 50 min, at least 60 min, at least 90 min, at least 2 hours, at least 3 hours, at least 4 hours, at least 5 hours, or at least 10 hours).

In some examples, the nanoparticles are in contact with the serum sample at a ratio (NP:sera) of about 5:1, 10:1, 15:1, 20:1, 25:1, 30:1, 35:1, 40:1, 45:1, or 50:1 by volume. In some examples, the nanoparticles are in contact with the serum sample at a ratio of 20:1 (NP:sera) by volume. In some examples, the nanoparticles are in contact with the serum sample at a weight-to-volume ratio (NP:sera) of about 0.5 mg:1 μL, 1 mg:1 μL, 1.5 mg:1 μL, 2 mg:1 μL, 2.5 mg:1 μL, 3 mg:1 μL, 3.5 mg:1 μL, 4 mg:1 μL, 4.5 mg:1 μL, or 5 mg:1 μL. In some examples, the nanoparticles are in contact with the serum sample at a weight-to-volume ratio (NP:sera) of about 2 mg:1 μL.

In some examples, disclosed herein is a method of generating one or more coronal protein-coated nanoparticles, comprising contacting one or more nanoparticles with a serum sample obtained from a subject for 15 min at room temperature, wherein the serum samples is obtained from a subject infected by influenza A virus, wherein the nanoparticle is a hyperbranched polyester (HBPE) nanoparticle, and wherein the nanoparticles are in contact with the serum sample at a ratio of 20:1 (NP:sera) by volume.

### EXAMPLES

The following examples are put forth so as to provide those of ordinary skill in the art with a complete disclosure and description of how the compounds, compositions, and methods claimed herein are used and evaluated and are intended to be purely exemplary of the disclosed subject matter and are not intended to limit the scope of what the inventors regard as their invention. However, those of skill in the art should, in light of the present disclosure, appreciate that many changes can be made in the specific aspects which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Polymeric Nanoparticles with a Sera-Derived Coating for Efficient Cancer Cell Uptake and Killing

Advances in nanotechnology hold promises for improving anti-cancer drug efficacy by delivering therapeutic cargo specifically to disease sites. However, achieving effective local accumulation of nanocarriers in tumors remains a challenge. Various strategies are ongoing to enhance the intratumoral concentration of nanomedicines. Taking advantage of the enhanced permeability and retention (EPR) effect, due to abnormal leakage of tumor vessels, is one approach for the passive targeting of nanomedicines. However, gaps between endothelial cells may not be responsible for the movement of particles from the vasculature into tumors, in part explaining why current approaches for nanoparticle drug delivery to cancer cells result in efficiencies as low as 0.0014%. Rapid removal of circulating NPs by the reticuloendothelial system (RES) is another factor that reduces tumor accumulation of systemically introduced nanomedicines. Anti-fouling approaches like the use of poly(ethylene glycol) (PEG) to modify the surface of NPs are used to enhance biocompatibility and increase circulation time. But the repeated administration of PEG-modified NPs can cause production of anti-PEG antibodies as part of the host immune response against PEGylated nanomedicines. PEG density and chain length are also factors that can hinder cancer cell uptake. Hence identifying novel anti-biofouling coatings that are not immunogenic is needed to overcome the limitations of current nanocarriers.

An emerging strategy for targeting NPs to tumors is modifying the nanoparticle surface to promote interactions with biological components that naturally target specific cells. In the presence of fluids like blood or cell culture media, NPs adsorb biomolecules forming what has been termed a “corona”. This corona is composed of proteins and possibly other biological molecules like lipids. A protein corona is likely formed by two distinct layers: a hard corona made from proteins with a strong affinity for the nanoparticle surface and a soft corona consisting of proteins that can transiently interact with NPs. The formation of a protein corona remodels the nano-bio interface and is thus a major factor in defining the pharmacological profile of nanomedicines. Key parameters such as blood circulation time, tissue biodistribution, biodegradation, hemocompatibility, toxicity, and others are affected by the biomolecules that form a protein corona on NPs. Such findings are the foundation for strategies to produce biomimetic NPs that integrate biological elements in nanoformulations. As an example, use of cell membrane-coated NPs was first examined using membranes from red blood cells (RBCs). Unlike PEGylation that is a “bottom-up” approach, coating NPs with cell membranes utilizes a “top-down” approach that is more facile and endows NPs with the characteristics of the cell membrane donor cell. In addition to RBCs, various other cell types can provide membranes to coat NPs, such stem cells, leukocytes, platelets or cancer cells. Despite advantages in immune evasion, improved biodistribution and circulation time, enhanced natural targeting, and being eco-friendly, the translation of cell membrane coating nanotechnology from the lab to the clinic remains challenging. There are unresolved issues with standardization of membranes source cells and large-scale production. The identity of the essential membrane proteins that improve tumor targeting of coated NPs is poorly characterized, which impedes reproducibility. Additionally, inherent problems exist with the use of RBCs and platelets, such as the lack of tumor targeting and the need for donor blood, and carcinogenic risk associated with cancer cells or exosomes.

An ideal solution is coating NPs with an optimal combination of proteins that confer a biological identity to the nanomedicines that is amenable to tumor accumulation. However, the knowledge needed to create such a nanoparticle coating is lacking. Increasing the understanding of the interaction of NPs with biomolecules is essential to advance the clinical application of biomimetic approaches for nanomedicines. The NPs themselves can be a key to achieving this end. If NPs selectively adsorb proteins and other biomolecules from surrounding fluid, these particles can enrich for high and low abundance factors important in the manipulation of the bio-interface that can be used to improve the targeting of nanomedicines to disease sites. Moreover, the physio-chemical features of NPs such size, shape, or hydrophobicity can be further modified to modulate corona formation and thereby lead to the identification of novel proteins that enhance the tumor targeting of nanomedicines.

This study used NPs formulated with a novel aliphatic and malonate-based synthetic polymer termed HBPE to investigate the effect of a serum-based protein corona on breast cancer cell uptake and drug delivery as compared to PEGylation. It was previously found that the three-dimensional (3D), globular HBPE polymer forms amphiphilic polymeric cavities for effective encapsulations of hydrophobic drug cargos. In the intracellular environment (e.g., esterases, acidic pH), the presence of ester linkages in the polymeric backbone yields small chain (2-5 carbon) length alcohols and weak acids. Such degradation byproducts are easily excluded from the body by the renal system as shown for other degradable polymers. This results in minimal toxicity and higher biocompatibility when HBPE-NPs are used in vivo. A synthetic polymer such as HBPE is also preferable to polymers formed using natural sugars, like chitosan, since synthesis is more reproducible and less likely to stimulate the immune system. Unlike other hyperbranched aliphatic polyesters, such as poly(glycolide) (PGA) that lack functional groups on the polymer backbone, the HBPE polymer forms carboxylated (COOH)-functionalized NPs that can be further functionalized using carbodiimide chemistry. Importantly, the HBPE polymer, unlike dendrimers, can be synthesized in one-pot and does not need multiple iterative steps. In aqueous solutions, the HBPE polymer self-assembles, with the hydrophobic areas internalizing, while exposing the hydrophilic areas containing the polar carboxylic acid groups. This property, unlike micro-emulsion methods, enables encapsulation of hydrophobic drug cargos using a water-based solvent diffusion method and is an advantageous feature of the HBPE polymer along with the capacity for surface functionalization, solubility in common polar solvents, and selective biodegradability at low pH or under enzymatic conditions.

Due the described features of HBPE-NPs, these particles can be an ideal platform for the enrichment and subsequent identification of biomolecules that facilitate tumor accumulation. In support, it was demonstrated that HBPE-based NPs delivered a hydrophobic peptide called CT20p in vitro and in vivo to tumor cells that led to cancer cell killing and tumor regression. Thus, HBPE-NPs can form a corona containing critical proteins that enhance the tumor accumulation of particles. To investigate this, comparisons were performed for the uptake of NS-treated HBPE-NPs and PEGylated (PEG) HBPE-NPs using monocytic, endothelial and cancer cell lines. A novel endothelial cell-based transwell assay was used to determine whether a pre-formed protein corona on HBPE-NPs can modulate interactions with an endothelial layer and then be subsequently taken up by cancer cells. Findings support that HBPE-NPs adsorb select sera components that enhance delivery of anti-cancer agents to tumor cells. Hence, HBPE-NPs can serve as a source for the discovery of new factors that, when used to coat NPs, can optimize the biological behavior of nanomedicines by positively influencing cancer cell-targeting capacity.

Optimization of Synthesis of HBPE Polymer and Characterization of HBPE-NPs

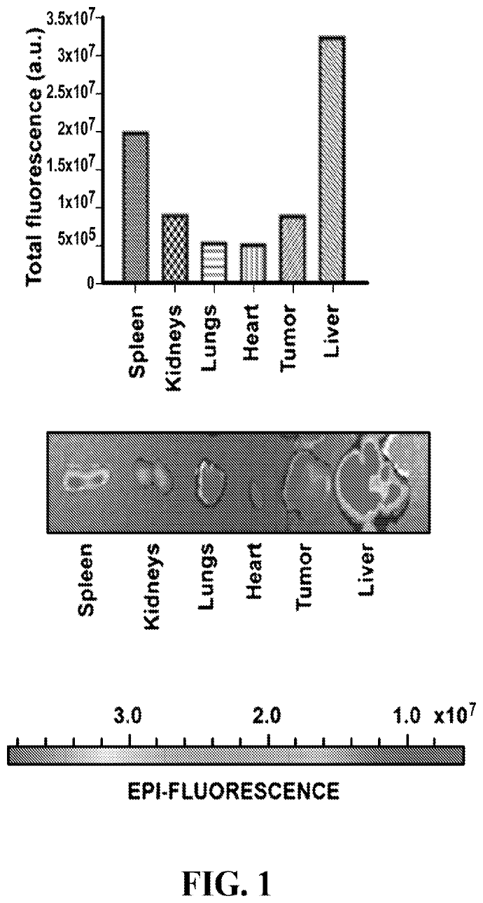

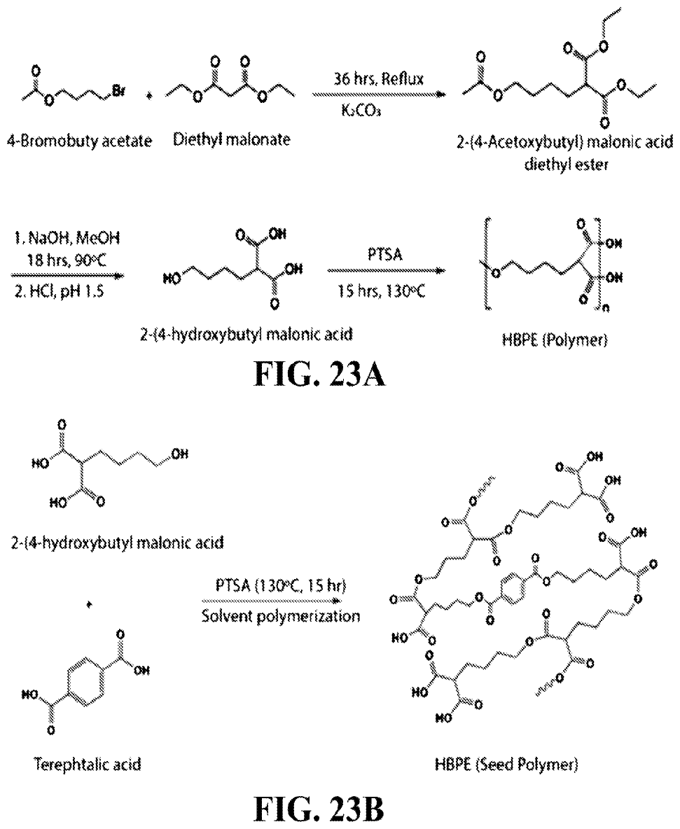

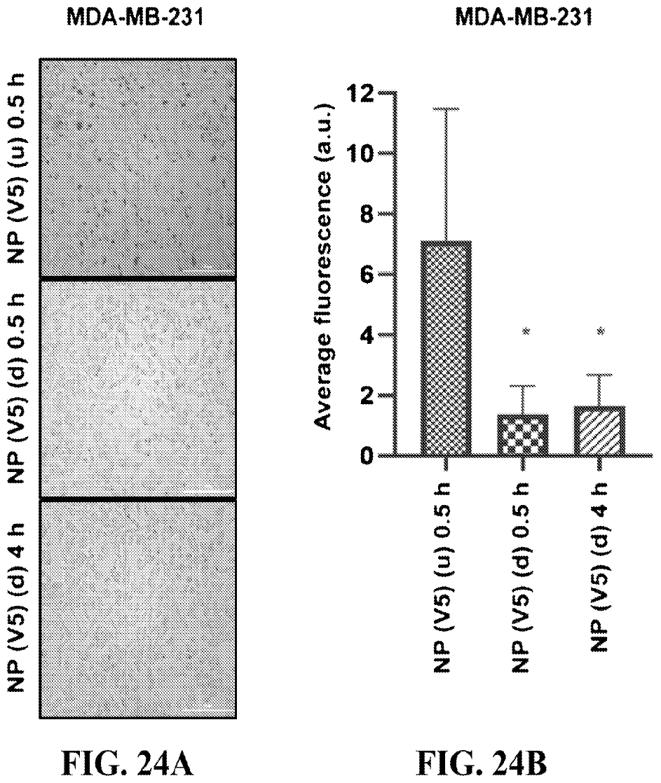

To address challenges with the delivery of cancer drugs to tumors, the HBPE polymer (Scheme 1A) was synthesized. An advantageous feature of the HBPE polymer is its aliphatic nature, consisting of hydrophilic carboxylic acids on its surface and hydrophobic hydrocarbons in its core. This property permits hydrophilic and hydrophobic drug encapsulation within the polymer's inner pores, facilitating solubility in aqueous environments. The AB2 monomer is designed to grow in three dimensions during polymerization, to form a highly branched polymer. The degree of polymerization of the HBPE monomer, and in turn its diameter and branching, can be altered through changes in reaction time during the synthesis, allowing for adjustment in polymer pore size to optimize drug encapsulation. In previous studies, HBPE-NPs were employed for delivery of a therapeutic peptide to regress breast and prostate tumors in mice. However, these studies also showed that the particles, even when PEGylated, were also taken up by the liver and spleen, which reduced bioavailability (FIG. 1). Since NPs of uniform size and shape are ideal for cargo delivery to cells, HBPE polymer synthesis was optimized to achieve uniform nanoparticle diameter and dispersity in several ways. First, in the monomer synthesis steps, column chromatography was replaced by vacuum distillation to reduce synthesis time and increase product yield of compounds 1 and 2 (FIG. 23A). Secondly, hydrochloric acid (HCl) concentration and dispersion rate was reduced to improve compound 2 recovery. Lastly, for polymerization, solvent volume was increased, and monomer was added dropwise to the solvent through a syringe pump at specific amounts and time intervals. Variants of this approach were tested to optimize branching of the polymer, which controls polymer molecular weight (MW) and resulting particle size and cargo-loading capacity. Two methods were evaluated: slow addition of monomer and use of terephthalic acid (1:20 ratio to monomer) as a seed for polymerization (FIG. 23B).

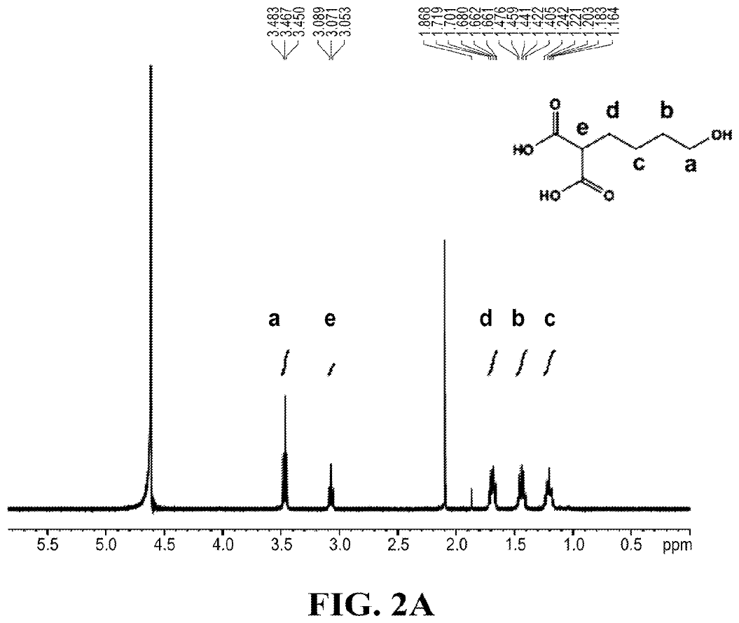

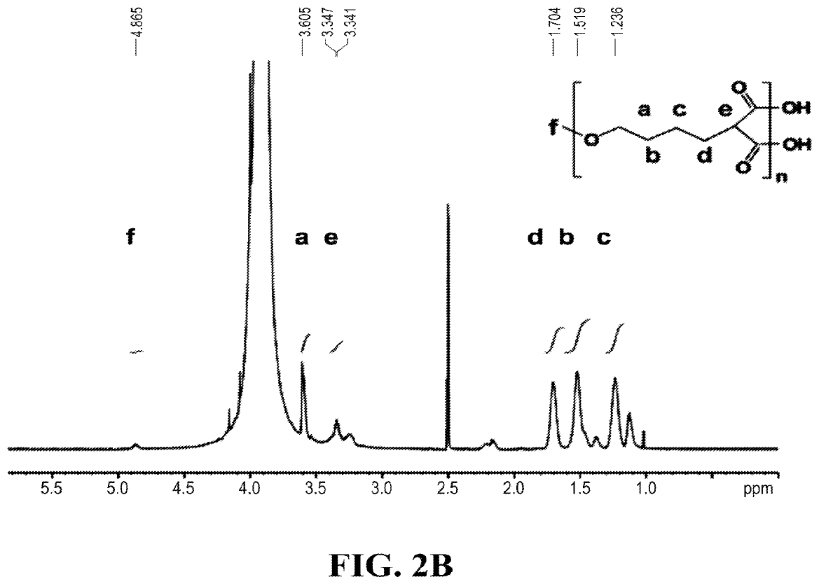

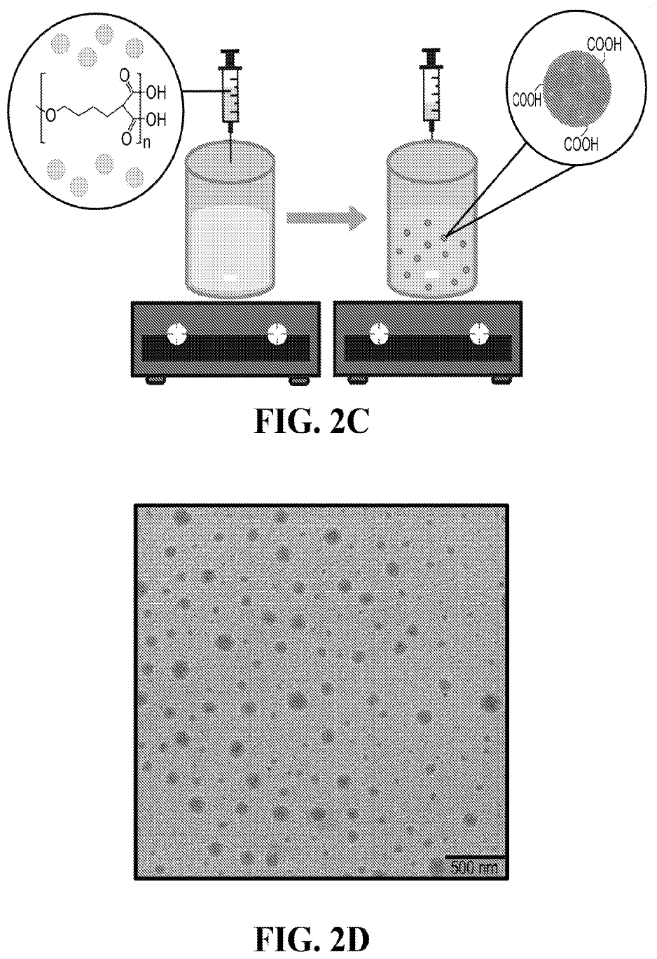

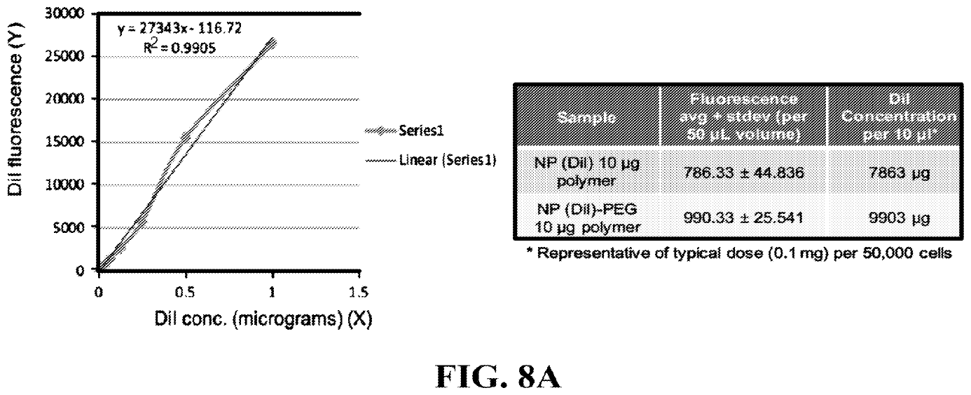

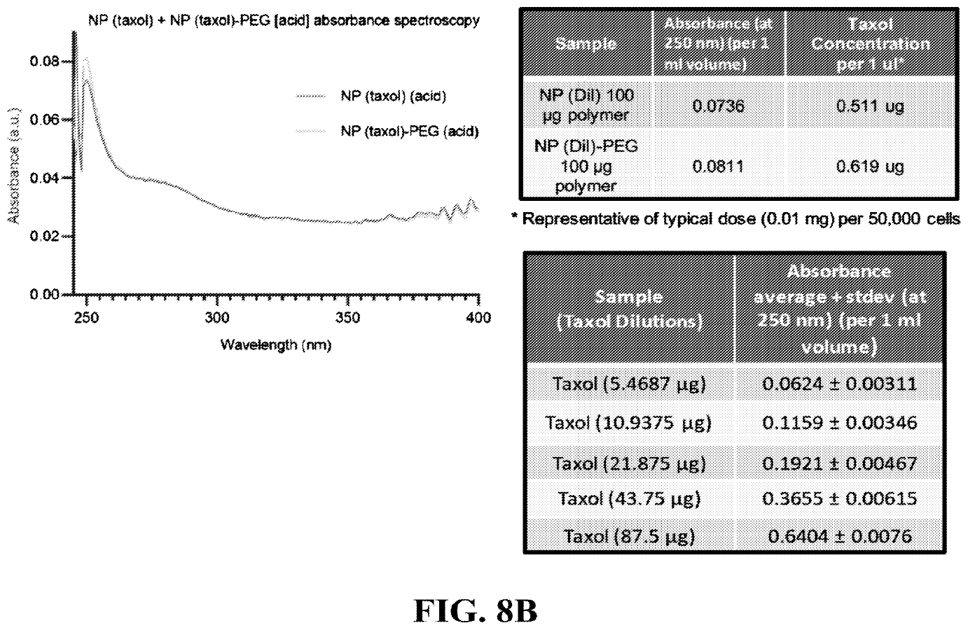

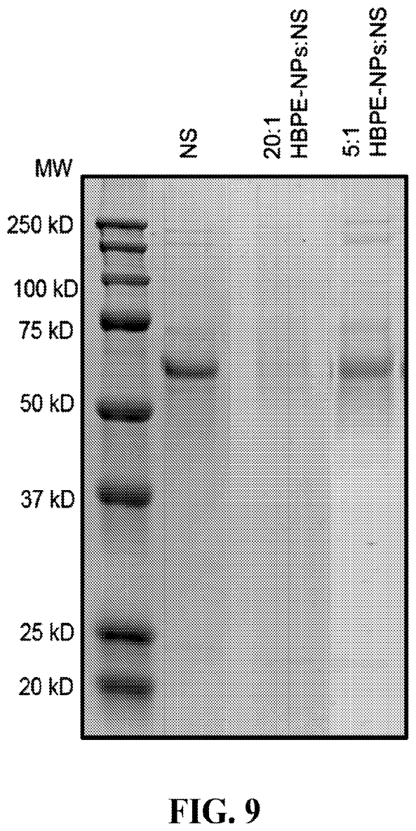

Nuclear magnetic resonance (¹H NMR) results for the HBPE monomer and polymer is shown (result for slow addition method shown, FIGS. 2A and 2B). Distinct peaks were observed with the correct number of segmentations and chemical shifts (ppm) for each hydrogen type, indicating successful monomer synthesis. After polymerization, NMR peaks widened and peak segmentation reduced, signifying monomer branching. In order to track NPs in cells or in vivo, fluorescent hydrophobic probes, 1,1′-Dioctadecyl-3,3,3′,3′-Tetramethylindocarbocyanine perchlorate (DiI) or 1,1′-dioctadecyl-3,3,3′,3′-tetramethylindotricarbocyanine iodide (DiR) was encapsulated within the HBPE-NPs as appropriate for detection. For dye encapsulation, the solvent diffusion method was used (schematic; FIG. 2C). All NPs had equivalent loading of dye by fluorometry (FIG. 8A). After dye-encapsulated NPs were formed, nanoparticle morphology, dispersity, and diameter were evaluated to ensure that NPs of uniform size and shape were produced. To examine this, transmission electron microscopy (TEM) was performed (FIG. 2D). Anhydrous NPs displayed a spherical and monodispersed nature and ranged in diameter between 100-160 nm, demonstrating that syringe pump-mediated control over polymerization was achieved. To evaluate hydrodynamic nanoparticle diameter, dynamic light scattering (DLS) was performed (Table 1). COOH-HBPE-NPs showed average diameter, dispersity [polydispersity index (PDI)], and zeta (ζ) potentials of 160 nm, 0.146, and −39 mV, respectively, indicating a desired diameter, stability and dispersity.

TABLE 1
DLS Data for NPs made from HBPE polymer.

| | Particle Size | | ζ- potential |
|---|---|---|---|
| NANOPARTICLE | (nm ± σ) | PDI ± σ | (mV ± σ) |
| COOH-HBPE-NPs | 160.1 ± 2.5 | 0.145 ± 0.068 | −39.1 ± 0.9 |
| COOH₍ₛ₎-HBPE-NPs | 150.0 | 0.147 ± 0.040 | −40.2 ± 1.5 |

Abbreviations:
₍ₛ₎terephthalic acid seed-based polymer;
PDI, polydispersity index

Protein Corona Formation and Inherent Toxicity of HBPE-NPs

An important factor influencing the biological identity of NPs is the formation of the protein corona. NPs can adsorb distinct proteins from biological fluids like cell culture media or sera, which in turn affect biodistribution when systemically applied. To study this, the coronae formed on HBPE-NPs treated with mouse NS was evaluated. A 5% volume-to-volume (or 20:1) ratio of NS to nanoparticle was used to coat HBPE-NPs. The sera to nanoparticle ratio and incubation time were determined by assessing the minimum sera volume and incubation time that allowed for a noticeable change in DLS diameter values upon sera addition to HBPE-NPs. To verify the presence of a protein corona, nanoparticle size was measured before and after NS exposure using DLS (Table 2). COOH-HBPE-NPs and PEGylated (PEG-HBPE-NPs) NPs were compared to assess the difference in nanoparticle surface modification upon corona formation.

TABLE 2
DLS Data for HBPE-NPs treated with NS

| | Particle Size | |
|---|---|---|
| Nanoparticle | (nm ± σ) | PDI ± σ |
| COOH-HBPE-NPs | 162.2 ± 3.8 | 0.185 ± 0.034 |
| COOH-HBPE-NPs + anti-IgG | 146.7 ± 3.9 | 0.182 ± 0.022 |
| PEG-HBPE-NPs | 224.6 ± 8.1 | 0.237 ± 0.004 |
| PEG-HBPE-NPs + anti-IgG | 206.1 ± 0.5 | 0.208 ± 0.025 |
| COOH-HBPE-NPs + 5% NS | 152.7 ± 3.0 | 0.123 ± 0.040 |
| COOH-HBPE-NPs + 5% NS + anti-IgG | 187.4 ± 1.8 | 0.095 ± 0.015 |

Abbreviations: PDI, polydispersity index

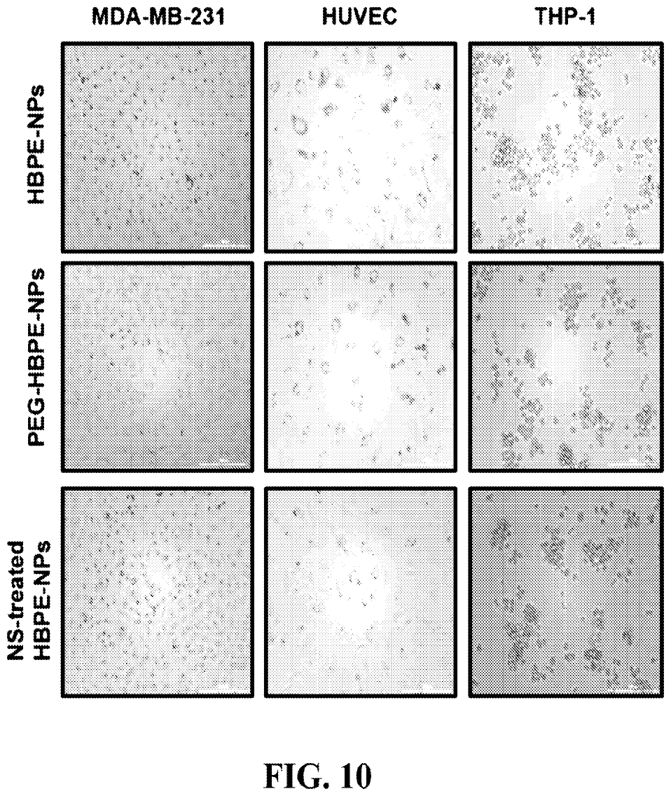

After NS treatment, COOH-HBPE-NPs decreased in average diameter by ˜5.8%. The size reduction of the HBPE-NPs can be due to proteins in the NS interacting with the particles' hydrophobic core and causing the branching of the HBPE polymer to constrict. To confirm that diameter changes in HBPE-NPs were due to adsorption of coronal proteins from NS, anti-IgG antibodies were employed using a protocol adopted from Zheng et al. to detect the immunoglobulin IgG associated with NPs. In principle, when HBPE-NPs capture sera proteins, such as IgG, antibodies to IgG can bind to the serum protein, increasing the size of the HBPE-NPs, which can be detected by DLS. After anti-IgG antibody addition, NS-treated COOH-HBPE-NPs increased in average diameter by ˜22.7%, respectively, as compared to NS-treated HBPE-NPs not incubated with anti-IgG antibody. Since the diameters of NS-treated HBPE-NPs increased upon anti-IgG antibody addition, but not the untreated COOH-HBPE-NPs (Table 2), HBPE-NPs formed a protein corona when exposed to NS. To confirm, the experiment determined that HBPE-NPs can concentrate serum proteins by analyzing the protein content of HBPE-NPs treated with NS at 20:1 or 5:1 volume-to-volume ratio by gel electrophoresis and staining with Coomassie Blue (FIG. 10).

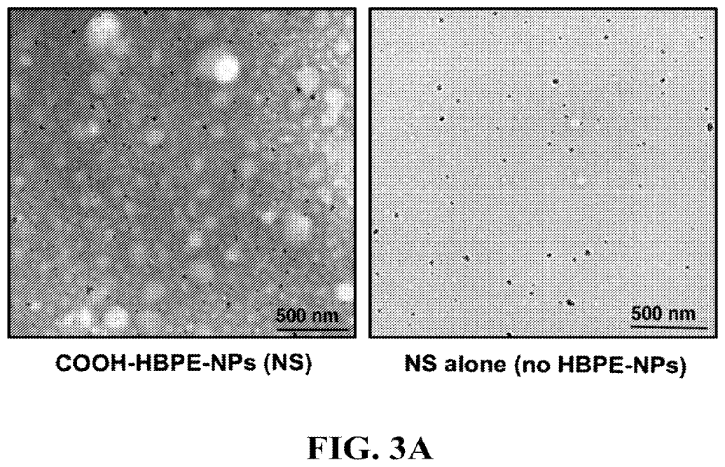

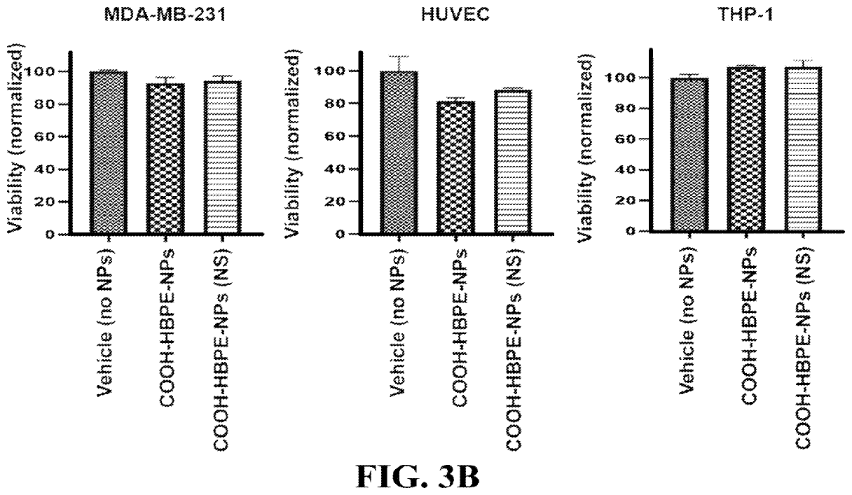

After DLS data confirmed the capacity of HBPE-NPs to form a protein corona with NS, corona formation was visualized by TEM. NS-treated HBPE-NPs resulted in observable nanoparticle agglomeration that was not seen with NS alone (FIG. 3A), confirming the presence of NS-treated HBPE-NPs. These data show that HBPE-NPs can form a protein corona that is based on the HBPE polymer's chemical structure. To ensure that cellular uptake would not be influenced by any adverse effects of NS-treated HBPE-NPs on MDA-MB-231, HUVEC, or THP-1 cells, an MTT (3-[4,5-dimethylthiazole-2-yl]-2,5-diphenyltetrazolium bromide) viability assay was performed (FIG. 3B). Treatment of cells with NS-coated HBPE-NPs after 24 hours showed no toxic effects, and viability was comparable to that of vehicle control (no NPs) or untreated HBPE-NPs. Note that these and subsequent experiments were performed in standard tissue culture media that contains 10% fetal bovine serum (FBS). Hence, untreated HBPE-NPs were included in all experiments as a control for the non-specific adsorption of proteins from FBS and to demonstrate that the pre-treatment of HBPE-NPs with NS, much like PEGylation, alters coronae formation, even in FBS-containing media.

Direct Uptake of NS-Treated HBPE-NPs by Monocytic, Endothelial and Cancer Cells.

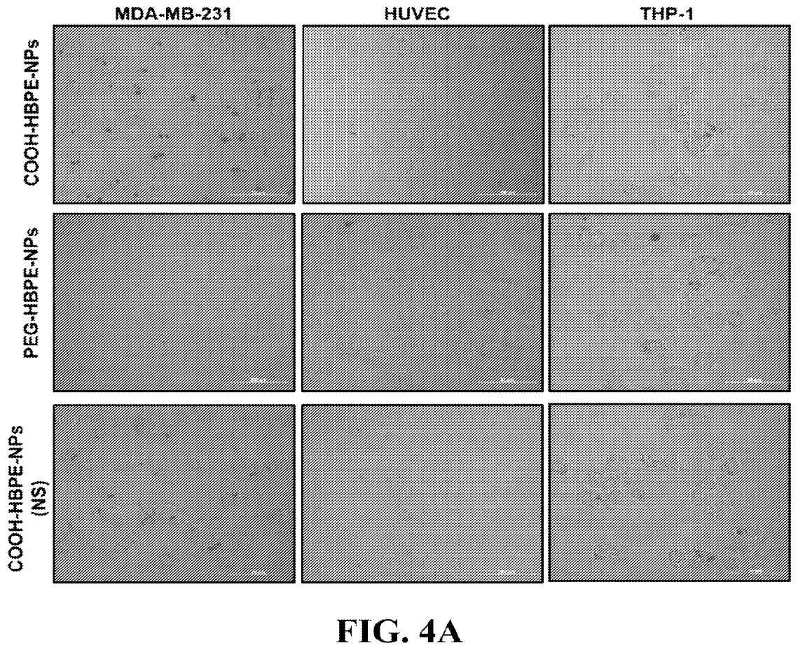

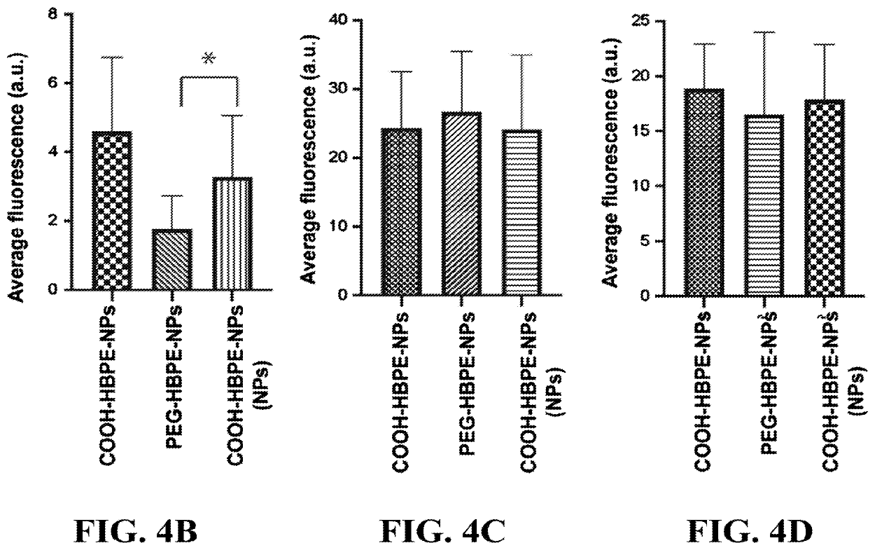

Once HBPE-NPs were confirmed to form a protein corona using DLS and TEM, in vitro studies were performed to evaluate whether the protein corona affected the ability of HBPE-NPs to be taken up by cells. Human-derived cell lines MDA-MB-231, HUVEC, and THP-1 were used, representing TNBC, endothelial, and monocytic cells, respectively. NS-coated HBPE-NPs loaded with DiI were incubated with MDA-MB-231, HUVEC, and THP-1 cells for 24 hours. PEGylated, DiI-loaded HBPE-NPs were included as a comparison to a standard in the field and untreated DiI-loaded HBPE-NPs used as a control for non-specific uptake of media-related proteins as indicated above. Note that nanoparticle preparations all had equivalent loading of DiI dye (FIG. 8A). To visualize internalization of DiI loaded HBPE-NPs by individual cells, laser confocal scanning microscopy was performed. To quantitate total fluorescence in a microscopic field of cells, digital microscopy coupled to a plate reader was used. Together, these two methods provide qualitative and quantitative evaluation of the nanoparticle uptake by cells. Hence, images were acquired by confocal (FIG. 4A) and digital (FIG. 10) microscopy to show mid-cell plane and total fluorescence, respectively. Fluorescence quantification was performed on digital images (FIG. 10) by averaging pixel intensity per cell. In MDA-MB-231 cells (FIGS. 4A and 4B), NS-treated-HBPE-NPs exhibited a significantly higher uptake (p<0.0001) compared to PEG-HBPE-NPs. With THP-1 cells and HUVECs, comparable uptake was noted between NS-treated and PEG-HBPE-NPs (FIGS. 4A and 4C-4D), indicating that NS-treatment did not enhance immune cell recognition or alter endothelial uptake. This result indicates that NS contains proteins not found in PEGylated particles that are advantageous for the delivery of HBPE-NPs to cancer cells, while evading monocyte uptake. The non-specific adsorption of media-derived proteins by untreated COOH-HBPE-NPs served as a positive control for uptake of particles by breast cancer cells and represented optimal outcomes achievable under in vitro cell culture. It is important to note that under in vivo conditions such untreated particles can be rapidly cleared and are not relevant in animal models; hence these are only used as controls for the in vitro studies.

Interaction of NS-Treated HBPE-NPs with Cancer Cells in an Endothelial-Based Transwell System.

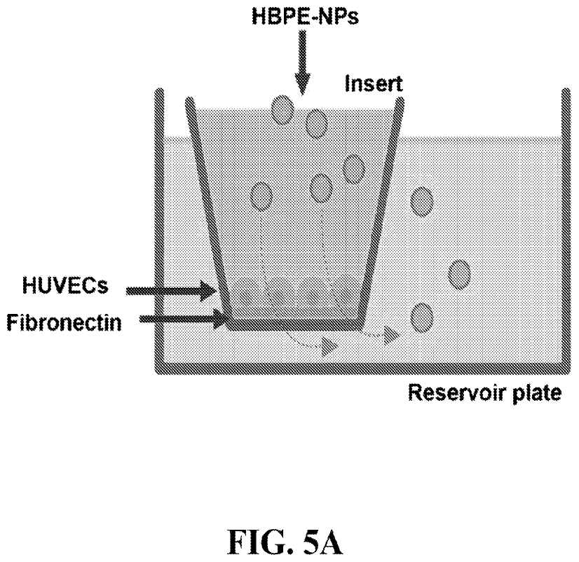

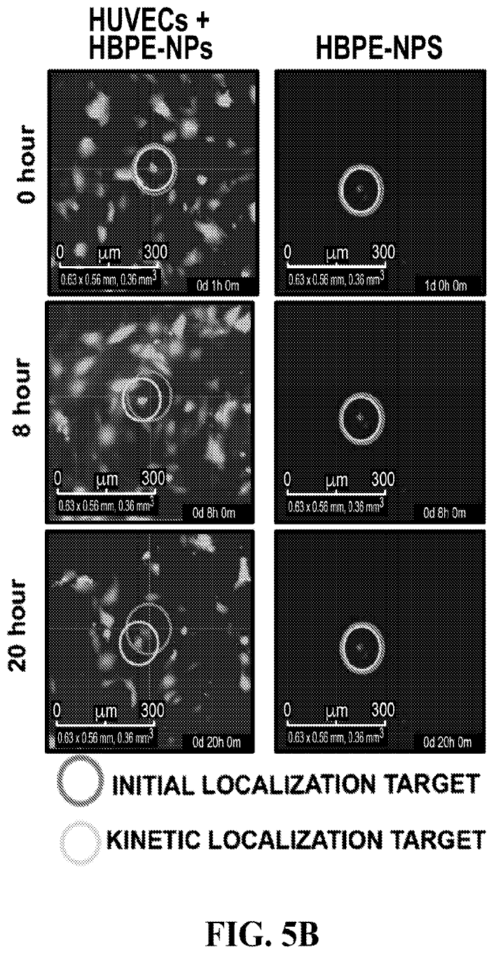

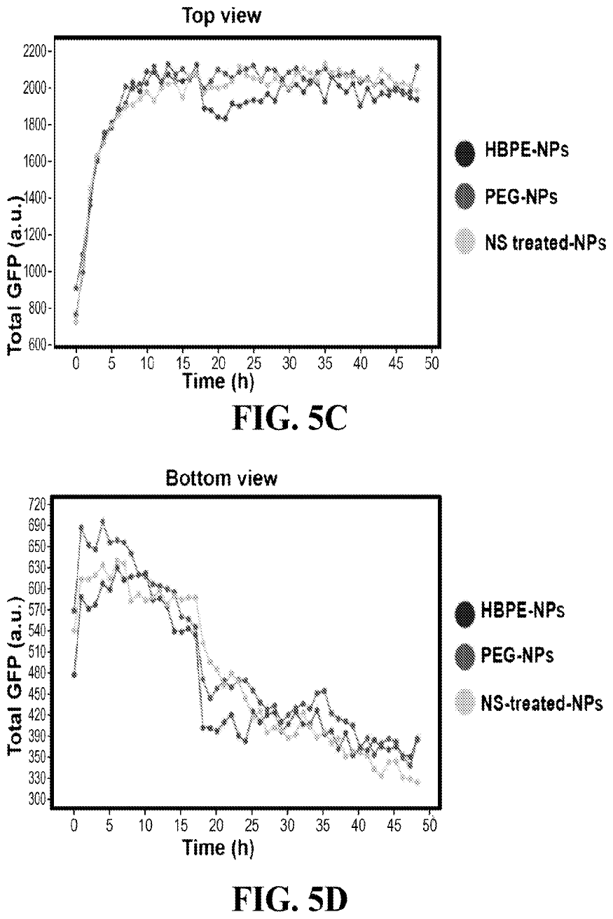

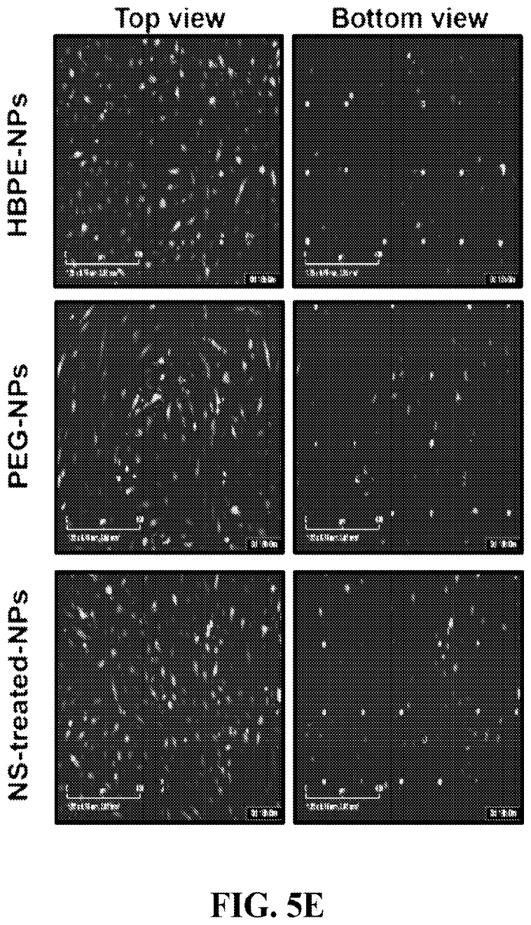

The in vivo administration of drug loaded HBPE-NPs may require that particles transit in the bloodstream and move through the vascular endothelium to reach tumor cells. Hence it is important to determine whether NS-treated HBPE-NPs that transit through endothelial cells can subsequently penetrate tumor cells. To investigate this, the Chemotaxis Transendothelial Migration (CTEM) protocol associated with the IncuCyte Live-Cell Analysis System was first used to study the interaction of HBPE-NPs with HUVECs. The CTEM is a two-part system in which an insert is treated with fibronectin and green fluorescent HUVECs (for tracking) are seeded up to 9,000 cells/well. The insert is placed inside of a reservoir plate with media (schematic, FIG. 5A) and loaded into the IncuCyte instrument, which scans (at 10× magnification) the top and bottom of the insert membrane over a two-day period at hourly intervals. The initial optimization of HUVEC seeding density showed that, over a 20-hour period, dye-loaded HBPE-NPs (red signal) did not move from the insert plate unless HUVECs (green signal) were present; hence free HBPE-NPs stay in the insert, likely trapped in the fibronectin layer, and move into the reservoir plate when facilitated by interaction with HUVECs (FIG. 5B). These results show that complete confluency of the HUVEC layer does not be needed to prevent free HBPE-NPs from entering the reservoir plate; this is further assessed in subsequent figures. To determine whether HBPE-NPs did not induce the migration of HUVECs, which can promote angiogenesis and tumor growth, DiI-loaded NS-treated HBPE-NPs and controls, PEG- and COOH-HBPE-NPs, were added to the insert containing HUVECs (at ˜80% confluency). Cells were imaged, above and below the insert, at 0.5- or 1-hour intervals for 24 or 48 hours, accordingly, for detection of green fluorescence. Total fluorescence in the green channel (above and below insert pores) was quantified (FIGS. 5C-5D). Since total green fluorescence (indicative of HUVECs presence) for all nanoparticle treatments was comparable above the pore layer and consistently decreased over time below the pores, this implied a similar rate of migration irrespective of nanoparticle presence. Hence the treatment of HUVECs with HBPE-NPs (NS-treated or controls) did not enhance migration in a manner that can promote subsequent tumor growth. Representative images of green fluorescent HUVECs present above and below pores of the insert are additionally shown (FIG. 5D).

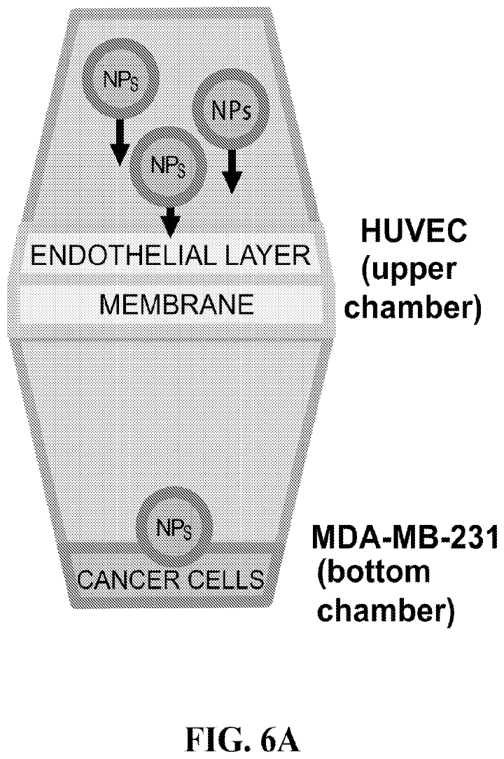

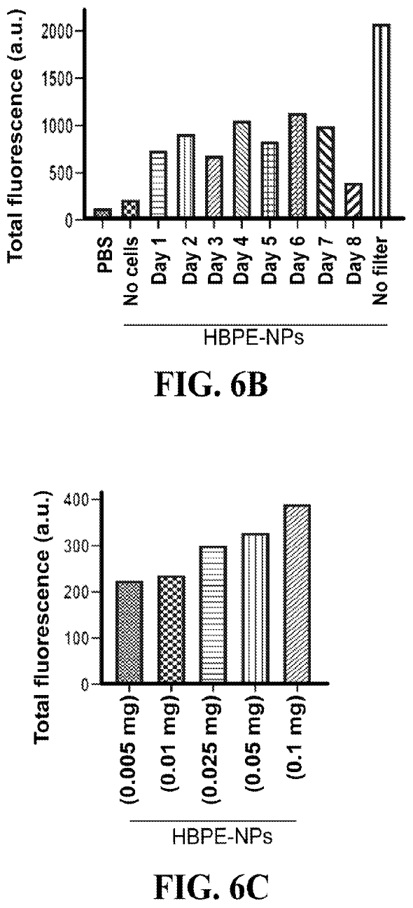

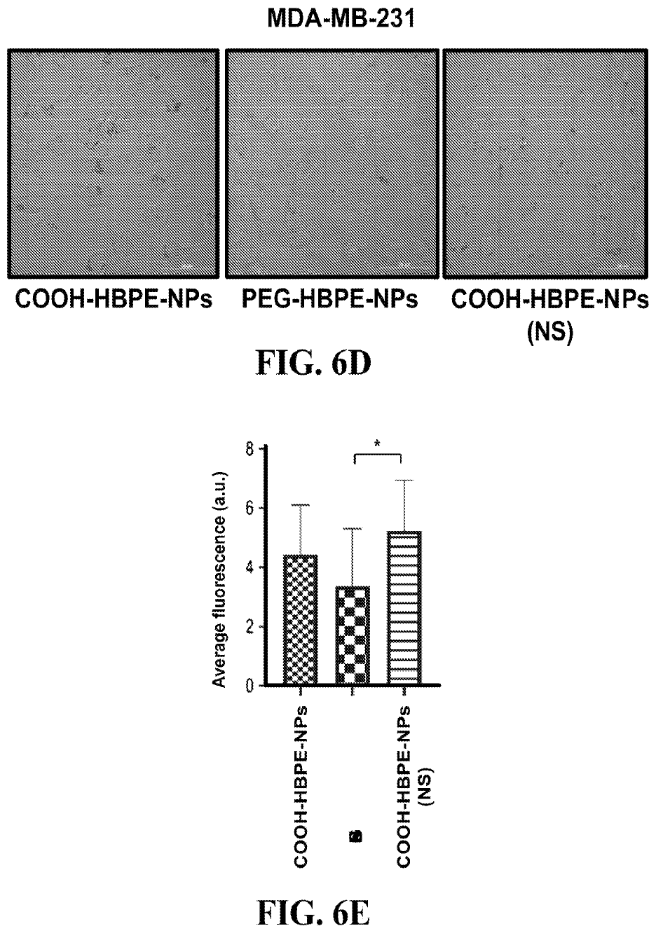

Cancer cell uptake of NS-treated HBPE-NPs was next evaluated utilizing a modified transwell system. The transwell system consists of an upper chamber containing a layer of HUVEC cells (initially plated at ˜80% confluency), a membrane that separates the chambers, and a lower chamber in which MDA-MB-231 cells are plated (schematic; FIG. 6A). In this way, aspects of the distribution behavior of HBPE-NPs moving through an endothelial layer into tumor tissue can in part be simulated in vitro. While this system does not replicate the fluid dynamics of blood, it enables the evaluation of the cellular interactions involved in transendothelial movement and cancer cell uptake of particles. To confirm that HUVECs were needed for the movement of HBPE-NPs between chambers, HUVECs were seeded in equal numbers per well and allowed to proliferate up to 8 days. HPBE-NPs were introduced into the top chamber from each (day 1 to 8), and fluid was collected from the bottom chamber for detection of fluorescent particles by digital imaging. Controls included phosphate-buffered saline (PBS), HBPE-NPs alone (negative control), and HBPE-NPs directly introduced into the bottom chamber (positive control). Few of the HBPE-NPs introduced into the upper chamber were detected in the lower chamber in the absence of HUVECS, which confirmed results observed with the CTEM protocol (FIG. 5B). In the presence of HUVECs, three- to five-fold higher amounts of fluorescent HBPE-NPs were detected in the bottom chamber, a result that was independent of cell proliferation (FIG. 6B).

After confirming that, in the absence of HUVECs, HBPE-NPs did not move effectively through transwell pores and determining that HUVECs were sufficiently confluent by 24 hours after seeding to promote the movement of NPs into the bottom chamber, these conditions were used for subsequent transwell experiments. Note that post-day 8, HUVECs began to die under these culture conditions—further supporting that live HUVECs were needed for the movement HBPE-NPs between chambers. Next determined is the optimal nanoparticle dose to detect the movement of HBPE-NPs through a layer of HUVECs. A dose range of 0.005 mg, 0.01 mg, 0.025 mg, 0.05 mg, and 0.1 mg (based on HBPE polymer concentration) was used to treat HUVECs for 24 hours. 0.1 mg served as a reference dose that was used in the previous in vitro experiments. Following treatment, NPs were collected in the bottom chamber, and DiI dye total fluorescence was measured. It was observed that an increase in bottom chamber fluorescence was directly proportional to the nanoparticle treatment dose listed above (FIG. 6C). After determining a limit of detection of approximately 0.005 mg of nanoparticle, a 0.1 mg nanoparticle dose was chosen for subsequent transwell studies.

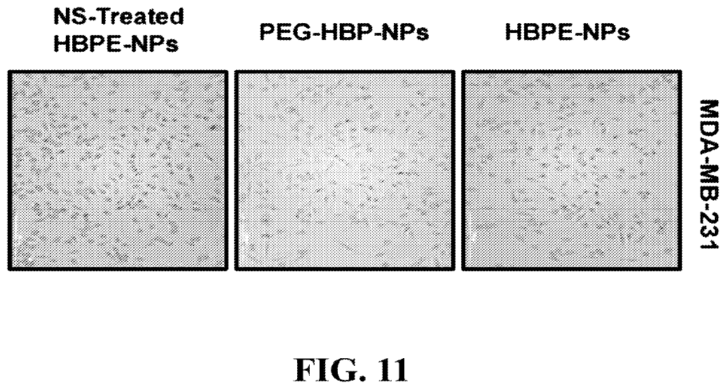

After optimizing the transwell experimental protocol, we determined whether NS-treated HBPE-NPs that initially interacted with endothelial cells can still be taken up by cancer cells. Controls included PEG-HBPE-NPs as a standard for comparison and untreated HBPE-NPs to capture the optimal uptake in culture media. After HBPE-NPs were incubated with HUVECs (upper chamber) for 24 hours, uptake of particles by individual MDA-MB-231 cells (bottom chamber) was visualized by confocal microscopy (FIG. 6D) and total fluorescence in a microscopic field of cells quantitated by digital microscopy coupled to a plate reader (FIG. 11, FIG. 6E). A statistically significant increase in the uptake of NS-treated HBPE-NPs by TNBC cells was observed as compared to PEGylated HBPE-NPs. These results suggest that the protein coronae formed on HBPE-NPs after treatment with NS still facilitated uptake by cancer cells, even after a prior interaction with endothelial cell monolayer.

Drug Delivery to Cancer Cells is Enhanced by NS-Treated HBPE-NPs.

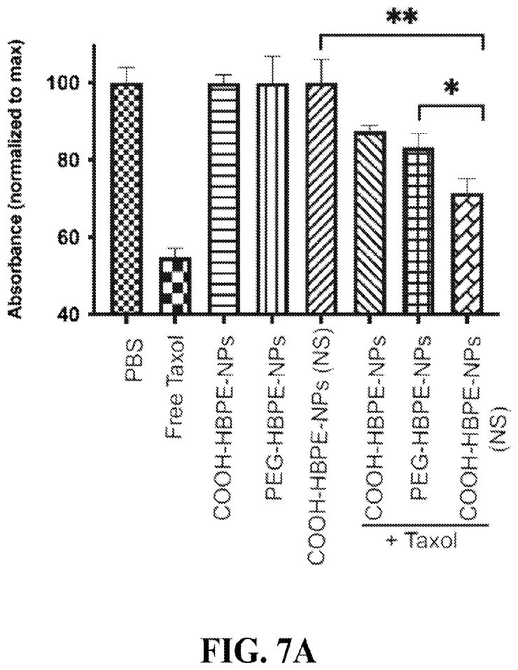

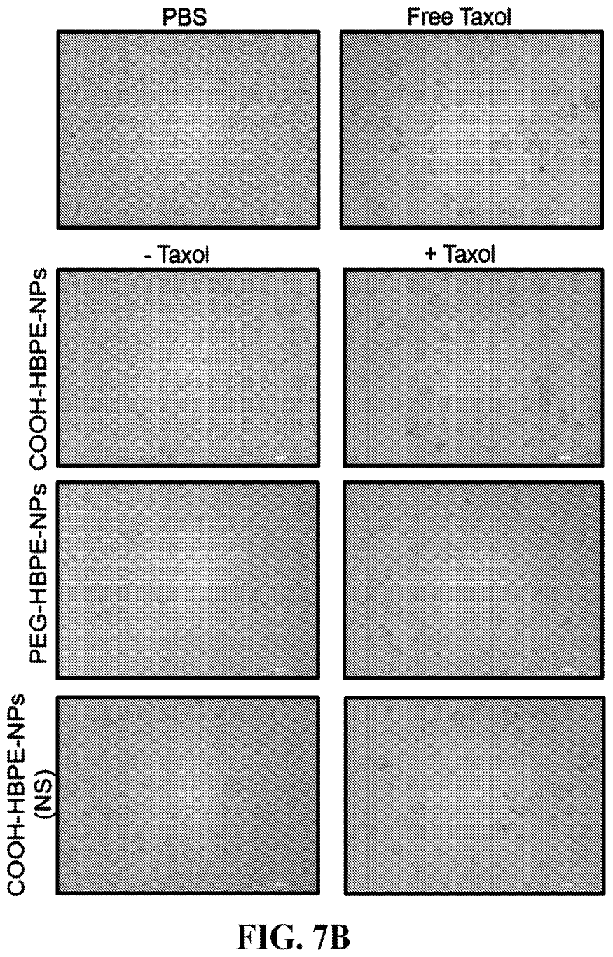

To evaluate the capacity of the NS-treated HBPE-NPs to deliver drug cargo to breast cancer cells, HBPE-NPs were encapsulated with taxol and determined that NPs were equivalently loaded with drug cargo (FIG. 8B; ˜0.5-0.6 μg taxol loaded per 0.01 mg HBPE polymer). IC50 dose of free taxol to be ˜50 nM (43 μg) was established. The capacity of NS-treated HBPE-NPs to deliver taxol to breast cancer cells was then compared as relative to PEGylated HBPE-NPs, using 0.01 mg dose of HBPE-NPs to observe relative differences in viability. All taxol-loaded nanoparticle preparations caused increased death of cancer cells as compared to drug-free HBPE-NPs (FIGS. 7A-7B), showing that HBPE-NPs can be effectively loaded with drug cargo. Based on the uptake data shown in FIGS. 4B and 6E, NS-treated HBPE-NPs can more efficiently deliver taxol to cancer cells. This was observed after 24 hours of treatment of MDA-MB-231 cells with taxol-loaded NS-treated HBPE-NPs. A statistically significant decrease in viability was noted as compared to taxol-loaded, PEGylated HBPE-NPs as well as taxol-loaded COOH-HBPE-NPs (FIG. 7A). This was confirmed microscopically using digital imaging (FIG. 7B). Moreover, HBPE-NPs achieved taxol-mediated killing at doses of taxol that were significantly than free drug.

The formation of the protein corona on polymeric NPs is a critical parameter that affects the performance of these particles when loaded with clinically relevant drugs like taxol. Characterization of this protein layer is thus important when developing new nanomedicines. While more is known on coronal formation using metallic NPs, much less is known about the protein corona the forms with polymeric NPs, and no information is available on the proteins that associate with NPs made using the malonate-based HBPE polymer described herein. This is the first study revealing that HBPE-NPs can enrich for select sera proteins that enhance the uptake of particles by cancer cells over PEGylated HBPE-NPs, even after an initial interaction with an endothelial layer, and deliver cytotoxic doses of a hydrophobic drug like taxol. Hence, HBPE-NPs are a promising platform for new discovery that advances the generation of biomimetic NPs.

Taxanes (e.g., paclitaxel, docetaxel, cabazitaxel) are a widely used class of anti-mitotic drugs for cancer treatment, but their application is characterized by severe off-target effects that include hypersensitivity reactions, peripheral neuropathy and other toxicities. As a result, in some patients the beneficial use of taxanes is limited. Nanocarriers solve this problem. An example is BIND-014, a polymeric nanoparticle loaded with docetaxel that targets tumors through PSMA. In clinical trials the toxicity profile of BIND-014 was found to be similar to free docetaxel and showed patient benefit in prostate cancer. Other drug delivery systems for taxanes include liposomes that are PEGylated to generate stealth nanocarriers. However, such particles can be subject to accelerated blood clearance and have issues with batch-to-batch reproducibility. Polymeric micelles encapsulating taxanes have also reached clinical trials (NANT-008 and NK105) and display increased tumor accumulation in preclinical studies and positive outcomes in clinical trials with varying degrees of adverse effects including hypersensitivity. Of the polymeric NPs, poly(lactic-co-glycoic acid (PLGA) is most commonly used but other natural (e.g., albumin) or synthetic polymers are also employed. Nab-paclitaxel (Abraxane) is the only FDA-approved albumin-based nanoparticle that delivers paclitaxel likely via albumen-driven transcytosis (e.g., transcellular transport). Despite pre-clinical and clinical trial successes, clinical evidence that use of NPs lead to increased tumor accumulation of taxanes is lacking. Part of the reason can be the need for a better understanding of the interface between NPs and the surrounding biofluids and how this affects biodistribution and cancer cell uptake. Herein it is shown that NPs, formed using HBPE, can have different cellular uptake outcomes, based on whether a particle is PEGylated or pre-treated with NS, and this impacts the delivery and subsequent cytotoxicity of taxol.

Being able to anticipate or predict how the formation of a protein corona on NPs can affect in vivo biological processing is a pressing need. In general, the major proteins found in the protein corona formed on NPs exposed to sera can either promote clearance by the RES (e.g., opsonins like complement), which is reduced by PEGylation, or have less affinity for cell surfaces and improve blood circulation (e.g., dysopsonins like serum albumin). Nano-liquid chromatography-tandem mass spectrometry revealed that the adsorbed proteins from normal human plasma on PLGA-NPs changed based on size, charge and composition of particles and showed positive and negative correlations between dysopsonins and opsonins. Common and unique proteins were identified such as albumin and immunoglobulins respectively. HBPE-NPs in this study were also able to enrich for common proteins and unique immunoglobulins, as shown by gel electrophoresis and DLS experiments with anti-IgG antibodies. To more directly assess the effectiveness of a protein corona derived from healthy sera, transferrin (Tf)-modified polystyrene NPs were treated with the plasma from normal individuals as compared to lung cancer patients and uptake by A549 lung cancer cells determined. Tf-NPs coated with normal sera were more effectively internalized by lung cancer cells. The negative effect of exposure to disease-derived biofluid was reversed by pre-coating these Tf-NPs with sera from healthy mice. Similarly, it was found that HBPE-NPs pre-treated with NS from mice, improved the delivery of taxol-loaded HBPE-NPs compared to PEGylated HBPE-NPs. Characterizing the proteome enriched on Tf-NPs exposed to normal plasma compared to lung cancer patient plasma revealed differences in major proteins: less albumin and more complement proteins in lung cancer plasma-derived NPs and higher levels of alpha-2 macroglobulin (A2M) in normal plasma derived NPs. These results show that protein coronas derived from healthy biofluids can improve the clinical applications of nanomedicines.

Further studies of the protein coronas of NPs are needed to investigate the dynamics of the nanoparticle proteome and fully develop new technologies to improve cancer drug delivery. One challenge is accurately assessing the composition of protein coronae given that the configuration of the nanoparticle-host serum interface can be a complex arrangement of different macromolecules or aggregations. This can lead to over or underestimation of the actual protein content adsorbed by NPs. Studies showed that composition of proteins bound to NPs can depend on the nanoparticle to sera concentration. Plasma variance among individuals is another factor that influences the interaction of NPs and can lead to the generation of personalized biomolecular coronas to control nanoparticle targeting. To this end, the physio-chemistry of HBPE-NPs can lead to discovery of novel sera-derived factors as indicated by these findings. This was the case with NPs formed using other polymers like PLGA or polycaprolactone (PCL) in which unique nano-proteome fingerprints were detected depending on the polymer used. PLGA-NPs bound human sera proteins with lower affinity compared to PCL-NPs, which adsorbed distinct proteins. The concept that NPs can be functionalized with natural materials, such as by protein adsorption, is emerging as a viable approach for improving bioavailability. Moreover, identifying coronal proteins can help trace the transport pathways of particles through epithelial and endothelial layers, and help reveal mechanisms of transcytosis. Using particles like HBPE-NPs to enrich for and discover both the high and low abundance proteins from sera or other relevant biofluids, as demonstrated in the data herein, is an important step to advance novel pre-coated NPs for therapeutic and diagnostic uses in the treatment and detection of cancer.

Utilizing NPs to improve drug delivery to tumors can enhance the efficacy of cancer therapies. To this end, the capacity of NPs to selectively adsorb proteins from biofluids, like normal mouse sera, can be used for discovery of novel factors to functionalize nano drug carriers for testing in pre-clinical cancer studies. This study investigated whether NPs formed using the HBPE polymer can adsorb components from NS that can endow the particles with cancer cell uptake capacity, that was as good, if not better, than a standard anti-fouling approach like PEGylation. Using a TNBC cell line, as well as endothelial and monocytic cells, it was found that NS-treatment increased the uptake of HBPE-NPs by cancer cells, as compared to PEG-HBPE-NPs, while not enhancing monocyte uptake. Hence, the coating on HBPE-NPs provided by treatment with NS can facilitate the internalization particles by cancer cells without augmenting immune clearance. NS-treated HBPE-NPs were inherently non-toxic and did not stimulate the migration of endothelial cells. The NS-derived corona formed on HBPE-NPs improved cancer cell uptake, even after an initial interaction with endothelial cells. Loading NS-treated HBPE-NPs with taxol revealed that these particles can efficiently deliver drug cargo to cancer cells, as compared PEGylated particles. These findings support the further investigation of sera-derived components enriched by HBPE-NPs to generate the next generation of biomimetic nanomedicines.

Material and Methods

Materials. For nanoparticle synthesis and preparation, 2-(N-morpholino)ethanesulfonic acid (MES) 5× buffer (pH 7.4) was purchased from Alfa Aesar (Haverhill, MA, USA). Acetone, dimethyl sulfoxide (DMSO), ethyl acetate, hydrochloric acid (HCl), isopropanol, methanol, petroleum ether, and sodium sulfate (Na₂SO₄) and other chemicals were obtained from Fisher Scientific (Waltham, MA, USA). DiI dye and DiR dye were purchased from Life technologies (Carlsbad, CA, USA). 4-bromobutyl acetate (BBA), acetonitrile, diethyl malonate (DEM), 1-Ethyl-3-(3-dimethylaminopropyl)carbodiimide (EDC), iodine crystals, N-hydroxysuccinimide (NHS), poly(ethylene glycol) 2-aminoethyl ether acetic acid 10,000 MW, potassium carbonate (K₂CO₃), p-toluenesulfonic acid (PTSA), silicone oil, sodium hydroxide (NaOH), and terephthalic acid were obtained from MilliporeSigma (Burlington, MA, USA). Purified deionized water was acquired through a Milli-Q purification system from MilliporeSigma.

For nanoparticle characterization, methyl sulfoxide-d6 (DMSO-d6) was purchased from Acros organics (Geel, Belgium) and anti-mouse IgG (Fab-specific) goat antibody was purchased from MilliporeSigma.

For cell studies, Ham's F-12K (Kaighn's) Medium, HUVEC (CRL-1730) cells, THP1 (TIB-202), and MDA-MB-231 (HTB-26) cells were obtained from ATCC (Manassas, VA, USA). Neutral buffered formalin (10%) was obtained from Azer Scientific Inc. (Morgantown, PA, USA). Dulbecco's Modified Eagle Medium (DMEM), Endothelial Cell Growth Supplement (ECGS), L-glutamine, PBS, penicillin-streptomycin solution (10,000 U/mL), and 0.25% trypsin (0.1% EDTA in HBSS) were purchased from Corning (Corning, NY, USA). FBS was obtained from Gemini Bio-Products (Sacramento, CA, USA). Heparin sodium salt from porcine intestinal mucosa was purchased from MilliporeSigma. 3-(4,5-Dimethylthiazolyl-2)-2,5-diphenyl tetrazolium bromide (MTT) was obtained from MP Biomedicals (Santa Ana, CA, USA). 4′,6-diamidino-2-phenylindole (DAPI), 10% neutral buffered formalin, and fibronectin bovine protein were purchased from ThermoFisher Scientific (Waltham, MA, USA). Paclitaxel (taxol equivalent) was obtained from Thermofisher Scientific.

For gel electrophoresis, Mini-PROTEAN TGX polyacrylamide gels and Precision Plus Protein Dual Color Standards protein ladder were purchased from Bio-Rad Laboratories (Hercules, CA, USA). β-mercaptoethanol was obtained from Millipore Sigma. Coomassie Brilliant Blue was purchased from ThermoFisher Scientific.

Compound (1): Synthesis of 2-(4-Acetoxy-butyl)-malonic acid diethyl ester. To a 2000 mL round bottom flask containing 1000 mL of acetonitrile was added K₂CO₃ (155.72 g), DEM (45.13 g), and BBA (50 g) added sequentially at molar equivalents of 1, 1.1, and 1, respectively, and mixed under stirring for 10 minutes at room temperature. The solution was refluxed for 36 hours to synthesize compound 1. A 500 mL separatory funnel was used to extract excess DEM and compound 1 in ethyl acetate, and discard K₂CO₃ and acetonitrile through deionized water. Excess DEM and compound 1 were filtered through Na₂SO₄ to remove water carryover. Rotary evaporation under vacuum was used to remove ethyl acetate at 70° C. Vacuum distillation was employed to remove excess DEM at 90° C. Thin layer chromatography (TLC) silica gel plates (MilliporeSigma) in a developing chamber solution of 10% ethyl acetate in petroleum ether were used with an iodine crystal chamber to verify compound 1 purification. NMR was performed to confirm compound 1 synthesis.

### Compound (2): Synthesis of 2-(4-Hydroxy Butyl)-Malonic Acid.

Purified compound 1 (5 g) was added to 200 mL of methanol and 110 mL of NaOH (2 M) in a 250 mL round bottom flask under stirring for 10 minutes at room temperature. The mixture was refluxed for 18 hours. Subsequently, 200 mL of HCl (1 M) was added drop wise, 10 mL at a time, under stirring, to the refluxed solution until an acidic solution (pH 1) was achieved. The solution underwent vacuum distillation for 18 hours at 90° C. to synthesize compound 2. To the distilled solution was added 35 mL of isopropanol, followed by centrifuging at 3000×g for 10 minutes. The precipitate, containing sodium chloride (NaCl), was discarded. The supernatant was extracted and put through rotary evaporation under vacuum to purify compound 2 from isopropanol and methanol. TLC was performed to verify compound 2 purification. NMR was carried out to confirm compound 2 synthesis.

### Compound (3): Synthesis of HBPE Polymer.

Compound 2 (monomer, 120 mg) was diluted to 90 mg/mL in DMSO and subsequently aspirated through a 3 mL BD Luer-Lok syringe (Fisher Scientific) using an 18-gauge syringe needle (Fisher Scientific). The syringe was placed vertically on a NE-300 syringe pump (New Era Pump Systems Inc., Farmingdale, NY, USA) and positioned within a rubber-capped neck of a 50 mL double-necked round bottom flask. To the 50 mL double-necked flask, 4.75 mL of DMSO and 250 μl of PTSA at 5 mg/mL were added prior to syringe insertion. The monomer to PTSA molar ratio was 100:1. The syringe pump was set to dispense the monomer solution at a 0.1 mL per hour rate. The reaction was run for 15 hours at 130° C. under nitrogen atmosphere. The polymer (compound 3) was dissolved in DMSO at 20 mg/mL. DMSO was removed through lyophilization in order to dissolve polymer in DMSO-d6 for NMR preparation. NMR was used to verify the polymer synthesis. The procedure for seed based HBPE polymer synthesis was adopted from the HBPE polymer synthesis method above with some modifications. All synthesis steps were identical for seed-based and non-seed-based HBPE polymer with the following exceptions. To the 50 mL double-necked flask, 4.75 mL of DMSO, 250 μl of PTSA at 5 mg/mL, and 500 μl of terephthalic acid at 12 mg/mL were added prior to syringe insertion. The monomer to PTSA molar ratio was 100:1. The monomer to terephthalic acid molar ratio was 20:1.

### Nanoparticle Synthesis, Drug Dye Encapsulation and PEG Functionalization.

HBPE-NPs were formed and encapsulated with dye or drug as follows. For dye loaded NPs, 0.001 mg of DiI or DiR dye in 100 μL of DMSO (HBPE-DiI/DiR NPs) was added to 10 mg of HBPE polymer. For drug loaded NPs, 2 mg of taxol was added to 10 mg of HBPE polymer. The mixture was then added dropwise, 10 μL at a time, to 4 mL of deionized water under vortex at 2000 rpm. For PEG functionalization, EDC (1.5 mg), NHS (0.5 mg) and PEG (1 mg) were weighed. Subsequently, EDC and NHS were dissolved in 100 μL of 1×MES buffer, while PEG was dissolved in 100 μL of deionized water. EDC, NHS, and PEG solutions were added individually to COOH-HBPE-DiI/DiR-NPs and incubated for 10 seconds, 3 minutes, and 4 hours, respectively, using a Rotamix (ATR biotech, Laurel, MD, USA). A Sephadex G-25 PD-10 Desalting Column (GE Lifesciences, Chicago, IL, USA) was used to remove excess dye, drugs, EDC, NHS, or PEG. NPs were then filtered through a 0.22 μm polyethersulfone (PES) membrane (MilliporeSigma). Afterwards, NPs were concentrated to 10 mg/mL, using an Amicon Ultra-4 Centrifugal Filter Unit (MilliporeSigma) centrifuged at 1600×g for 15-minute cycles. All encapsulation and functionalization procedures were performed at room temperature.

To verify uniform dye and taxol encapsulation in NPs, fluorescence and absorbance quantifications for DiI and taxol, respectively, were performed. To assess DiI encapsulation, a calibration curve was established with serial dilutions of DiI (Fig. S1A). Then, 10 μg of COOH- or PEGylated DiI-encapsulated NPs were dispensed in an HCl-acidified PBS solution (pH=4) and incubated for 6 hours at room temperature to release encapsulated DiI upon HBPE polymer ester hydrolysis. DiI fluorescence was measured with a Cytation 5 Cell Imaging Multi-Mode Reader at 531 nm excitation and 593 nm emission wavelengths. To assess taxol encapsulation, a calibration curve was established with serial dilutions of taxol (Fig. S1B). Then, 10 μg of COOH or PEGylated taxol-encapsulated NPs were incubated in an HCl-acidified PBS as above to release taxol and absorbance (UV/Vis) was read at a 250 nm wavelength using a Beckman Coulter DU 800 Spectrophotometer (Brea, CA, USA). Trendline equations of the serial dilutions for estimation of encapsulated DiI or taxol concentration in the NPs was performed, with the exception of the trendline's y-axis value being absorbance for taxol and fluorescence for DiI.

### HBPE Polymer and Nanoparticle Characterization.

Nuclear Magnetic Resonance (NMR)._NMR spectra were recorded on Bruker AvanceIII 400 MHz and Varian VNMRS 500 MHz spectrometers with solvent signal used as an internal reference. Samples in DMSO-d6 were calibrated with the solvent, and samples in D₂O calibrated with the CH₃ peak of residual undeuterated acetic acid. The following abbreviations were used to explain the multiplicities: t=triplet, q=quartet, quint=quintet, br=broad singlet or broad multiplet.

HBPE monomer, 1H NMR (400 MHz, D2O, δ ppm) δ=3.47 (t, J=6.6 Hz, 2H), 3.07 (t, J=7.2 Hz, 1H), 1.69 (q, J=7.7 Hz, 2H), 1.44 (quint, J=7.2 Hz, 2H), 1.20 (quint, J=7.8 Hz, 2H).

HBPE polymer, 1H NMR (500 MHz, DMSO-d6, δ ppm) δ=4.87 (br, 1H), 3.60 (br, 2H), 3.34 (br, 1H), 1.70 (br, 2H), 1.52 (br, 2H), 1.24 (br, 2H).

Transmission Electron Microscopy. Uncoated or serum-coated NPs (0.05 mg) were dispensed onto a 400-mesh copper grid (Ted Pella Inc., Redding, CA, USA). Excess solution was removed with a Kimwipe (Kimberly-Clarke, Iriving, TX, USA) and left to air-dry overnight at room temperature. Grids were imaged with a JEOL TEM-1011 (JEOL Ltd., Akishima, TYO, Japan) microscope at 100 kV and 6,000 magnification.

DLS Analysis and IgG detection. NPs (0.1 mg) were dispersed in 800 μL of deionized water in a folded capillary Zetacell (Malvern Panalytical, Worcestershire, UK). Nanoparticle hydrodynamic diameter and zeta potential were then measured with a Malvern Zetasizer ZS90 (Malvern Panalytical) instrument at room temperature. Regarding serum- and antibody-related experiments, a 20:1 volumetric ratio of 0.1 mg of NPs to mouse serum, or 2 mg/mL of IgG antibody, was used. NPs were incubated with either serum alone, or serum and subsequently antibody, for 15 minutes each. Incubations were done under gentle agitation at room temperature. All diameter and zeta potential values are averages of three replicates.

Cell culture. MDA-MB-231 TNBC cells were cultured in DMEM supplemented with 10% FBS, 2 mM L-glutamine, and 1× penicillin-streptomycin. HUVEC endothelial cells (Cytolight Green) (Essen BioScience) were cultured in F-12K media supplemented with 10% FBS, 2 mM of L-glutamine, 1× penicillin-streptomycin, 56 mg of heparin sodium salt, and 15 mg of ECGS. THP-1 cells were cultured in RPMI-1640 Medium supplemented with 2-mercaptoethanol 0.05 mM, 10% FBS2 mM of L-glutamine, and 1× Penicillin-Streptomycin. All cell lines were limited to a low number of passages and incubated in 5% CO2 at 37° C.

MTT viability assay. For cell viability assays, either deionized water (vehicle) or 0.1 mg of HBPE-NPs was used. Treatments were dispensed in 96-well culture plates, seeded with 0.5×10⁴ MDA-MB-231, HUVEC or THP-1 cells at 60% confluency. Culture plates contained 100 μL of media per well. 24 hours after treatment, 0.05 mg of MTT reagent was dispensed in each treated well and incubated for 4 hours in 5% CO₂ at 37° C. Media was then removed and replaced with 100 μL of DMSO. Culture plates were shaken for 15 minutes at 800 rpm. Absorbance was measured at 570 nm using a Cytation 5 Cell Imaging Multi-Mode Reader (BioTek, Winooski, VT, USA). Deionized water served as a negative control.

Cell Uptake Studies. 0.5×10⁴ MDA-MB-231, HUVEC or THP-1 cells were seeded in 96-well culture plates containing 100 μL of media per well. For pre-coating NPs with sera, a 20:1 volumetric ratio of 0.1 mg of NPs to NS was used. NPs were incubated with NS for 15 minutes. Incubations were done under gentle agitation at room temperature. Cells were grown until 60% confluency and incubated with 0.1 mg of NS-treated, COOH-HBPE-NPs, or PEG-HBPE-NPs. All NPs were loaded with DiI dye. Cells were incubated with NPs for 24 hours in 5% CO2 at 37° C. Afterwards, cells were washed with PBS, followed by fixation in 10% neutral buffered formalin for 10 minutes, and washed for a second time with PBS. Cells were imaged with a Zeiss LSM 710 confocal microscope (Carl Zeiss AG, Oberkochen, Germany). Analysis of intracellular fluorescence representative of particle uptake per individual cell was performed with ZEN blue software. To assess cellular uptake of fluorescent particles in a population of cells, digital imaging coupled to a fluorescent plate reader was performed with the Cytation 5 Cell Imaging Multi-Mode Reader. Total fluorescence per field of cells was captured and analyzed with ZEN blue software.

Chemotactic Transendothelial Migration (CTEM) Protocol. To evaluate nanoparticle transmigration over time, an IncuCyte S3 Live-Cell Analysis System (Essen Bioscience Inc, Ann Arbor, MI, USA) was utilized. HUVECs (Cytolight Green, Essen BioScience) were grown to 80% confluency in the upper culture plate of a fibronectin-coated ClearView 96-well chemotaxis plate (Essen BioScience). PBS was dispensed in the wells of the chemotaxis plate's bottom culture plate. Cells were then treated with 0.1 mg HBPE-NPs, PEGylated, or HBPE-NPs pretreated with NS as above. Directly after treatment, cells were imaged at 0.5- or 1-hour intervals for 24 or 48 hours, respectively, using a phase, green fluorescence, and red fluorescence channels. Time courses of red fluorescence total area and green fluorescence total count were quantified and graphed using the IncuCyte's chemotaxis software (Essen BioScience). Images and time course videos were additionally created using this software.

Modified Transwell Assay. 3×10⁴ HUVEC cells were seeded per well in a polycarbonate, 8 μm pore-sized, Millicell-24 Cell Culture Insert Plate (MilliporeSigma). Wells contained 500 μL of media and HUVEC cells were incubated for 48 hours in 5% CO₂ at 37° C. Afterwards, the HUVEC culture plate (upper chamber) was placed over a 24-well glass bottom culture plate (lower chamber) (Cellvis, Mountain View, CA, USA), containing MDA-MB-231 cells, to create an in vitro transwell system. 1.5×10⁴ MDA-MB-231 cells were seeded and grown to 60% confluency in 500 μL of media per well. Then 0.1 mg HBPE-NPs, PEGylated, or HBPE-NPs pretreated with NS as above (DiI loaded) were dispensed per well in the upper HUVEC chamber. Cells were incubated with NPs for 24 hours in 5% CO₂ at 37° C. MDA-MB-231 cells were then fixed with 10% formalin, stained with DAPI, and then imaged with a Zeiss LSM 710 confocal microscope (Carl Zeiss AG, Oberkochen, Germany). Analysis was performed with ZEN blue software. To assess total fluorescence, cells were also imaged with a Cytation 5 Cell Imaging Multi-Mode Reader and analyzed with ZEN blue software.

Mouse studies. For nanoparticle distribution studies, 8×10⁵ MDA-MB-231 luciferase-expressing (Luc) cells were orthotopically injected into the mammary fat pad of a 6-week-old Fox1-nu/nu (nude) female mouse. After the mouse's tumor reached ˜1000 mm³, the mouse was injected with 1 mg of DiR loaded, PEG-HBPE-NPs through the tail vein. After 7 hours post-injection, the mouse was euthanized, and its organs were harvested. Organs were imaged for DiR fluorescence using an IVIS Lumina S5 in vivo imaging system (PerkinElmer, Waltham, MA, USA). Fluorescence was quantified using Living Image software. For blood collection studies, C57BL/6 female mice, 2-3 months old, were used. Blood was collected through a terminal cardiac puncture and harvested in 1.5 mL Eppendorf tubes (Eppendorf, Hamburg, Germany), left to clot at room temperature for 1 hour, and then centrifuged at 13,400 rpm for 5 minutes with an Eppendorf Minispin (Eppendorf). All animal studies were approved by and performed under the University of Central Florida Institutional Animal Care and Use Committee (IACUC) guidelines.

Statistical analysis. Significance was determined conducting comparisons between two experimental datasets, as example, using a parametric two-tailed unpaired T-test with Welch's correction. At a confidence level of 95%, p-values <0.05 were considered significant as indicated in the figure legends. Statistical analysis was performed using GraphPad Prism 8 software.

### Example 2. Macromolecules Absorbed from Influenza Infection-Based Sera Improve Cancer Cell Uptake of Polymeric Nanoparticles

Triple-negative cancers (TNBC) account for ˜10-20% of breast cancer cases. TNBCs are more difficult to treat due to their lack of estrogen (ER), progesterone (PR), and human epidermal growth factor receptor 2 (HER2) receptors. TNBCs are also more prone to metastasis than other breast cancer classifications. TNBCs can develop resistance to current therapeutics. Additionally, treatments can be associated with issues of tumor-specific delivery, severe off-target effects, overactivation of the immune system, and low tumor entry (˜0.7%). Unfavorable outcomes may be reduced via NP-mediated administration.

A NP's flexible nature has great potential for enhancing current therapies and creating new ones. To demonstrate, NP exteriors can be designed with oligonucleotides, peptides, antibodies, lipids, cell-mimicking membranes, and other assorted compounds. Additionally, NPs can be designed to deliver a wide range of cargo. To illustrate, cargo can include anti-cancer peptides, tumor-infiltrating lymphocyte adjuvants, and ferromagnetic tumor imaging agents. Presently, there are eight FDA-approved nanoformulations for cancer therapy. These therapies employ liposomes, albumin-conjugates, and polymer-based NPs. For instance, Abraxane is an albumin-conjugated doxorubicin NP approved for breast cancer. NPs of albumin conjugated to paclitaxel (taxol) have also shown promise in enhancing overall survival rates in breast cancer patients. Clinical trials for TNBC therapy are investigating NPs modified with surface ligands for hyaluronan, CD44 and transferrin cancer receptors, among others. Therapeutic efficacy of these strategies can be limited due to tumor heterogeneity, which results in non-uniform and suboptimal levels of targetable receptors. Another hindrance may be attributed to similar treatment outcomes associated with receptor-specific (active) and passive targeting approaches. NP trafficking behavior, prior to reaching tumors, may be a factor.

It is estimated that less than 1% of NPs that are initially injected in mice are capable of reaching tumors. As a result, high delivery efficiency may be required for drugs to be effective. Severe side effects may stem from excessive drug distribution to healthy tissues. To overcome delivery insufficiencies, greater understanding of how NPs interact with biological fluids and tissues is needed. A controversial means regarding how NPs access tumors is through the enhanced permeability and retention (EPR) effect. The EPR effect relates to NPs transitioning in a passive non-specific manner from the blood to malignant tissue. This transition is believed to occur through large random endothelial gaps in vasculature. These gaps result from tumor-induced blood vessels that develop in a disorganized immature manner. Recently, evidence of gold NPs primarily using endothelial transcytosis rather than the EPR effect was demonstrated. Large endothelial gaps were present in only 0.048% of the vasculature's surface area. Transcytosis involves movement through a cell, instead of between cells.

Constructing NPs that can access tumors through transcytosis can increase drug transport. Endothelium crossing is a requirement for iron and low-density lipoprotein (LDL) passage through the blood-brain barrier. It is also necessary for IgG to cross the placenta for fetal development. Furthermore, white blood cells, termed leukocytes, can traffic through endothelium to access infected tissue through extravasation. Extravasation involves leukocytes interacting with a series of proteins shed in the blood to aid endothelial binding and passage. Endothelial-mediated NP delivery to cancers has lately been explored. In glioma, NP migration past endothelia is enhanced through insulin, glutathione, angiopep-2, transferrin, and interleukin-13 receptor targeting. NPs have also exhibited improved endothelial crossing via arginine-glycine-aspartate (RGD) and urokinase receptor affinity in pancreatic cancer.

Tumor distribution can also be influenced by proteins NPs interacts with. When NPs are exposed to biological fluid, their surfaces become coated with a variety of molecules. Molecules, such as metabolites, lipids, and proteins have shown affinity for NPs. Biofluid interaction can lead to multiple layers of proteins forming on NP surfaces, termed the corona. Corona layers can be classified as hard or soft. Hard coronas constitute high-affinity proteins to NP surfaces that mostly remain stably adsorbed to NPs. Soft coronas consist of low-affinity molecules that are dynamically exchanged. Corona formation leads to NPs forming a biological identity, characteristic of the type of molecules they're exposed to. Coronas can be mostly composed of common sera proteins like albumin, apolipoprotein, hemoglobin, and alpha-2-macroglobulin. Coronas can also be enriched with lesser abundant proteins, such as complement C8. NP size, shape, charge, and functionalization can also influence corona formation. PEGylation of PLGA NPs reduce complement presence in the corona. When PLGA NP size was increased from 100 nm to 200 nm, 200 nm NPs had apolipoprotein C-III, while 100 nm NPs did not. On the other hand, 100 nm NPs uniquely had immunoglobulin light chain lambda. Differences in PLGA nanoformulations affect on cell uptake were not explored, but authors suggested reduced complement within the corona could prolong circulation time in vivo. Enhanced apolipoprotein presence can be advantageous for cancer targeting.

Corona composition can also affect immune cell uptake. Liposomes pre-coated with human plasma exhibited a reduction in leukocyte uptake compared to non pre-coated controls. The authors surmised that pre-coating reduced the leukocytes ability to interact with coronal fibrinogen. NP coronas can also be affected by extrinsic factors, outside the physical properties of NPs. When NPs migrate through the body they interact with a variety of different fluids, like blood and lymph. Concentrations of particular coronal proteins can vary based on the protein source. Iron oxide NPs were shown to possess apolipoprotein B-100 and complement C3 when exposed to human lymph fluid versus human sera.

Protein concentrations can also differ in normal and disease-associated sera. In biomarker study of breast cancer patients, complement C3a and apolipoprotein H were elevated in their sera compared to healthy individuals. Differences in these proteins may alter a NPs interaction with immune cells and lipids, and consequently, affect their biodistribution. Moreover, proteins concentrations in lung lavages from influenza-infected mice changed during different days after infection. Haptoglobin, which was elevated 26-fold on day 5 post-infection compared to pre-infection, was elevated only 4-fold 14 days after infection compared to pre-infected lavages. Other proteins, such as plasminogen, remained relatively stable throughout infection.

Biological identity can influence in vivo NP behavior as well. For example, the circulation time of poly (lactic-co-glycolic acid) (PLGA) polymeric NPs can change based on apolipoprotein E concentration in their coronas. Additionally, biological identity can vary when NPs are incubated in distinct fluids. Magnetite NP coronas formed distinct protein patterns when incubated with mouse blood ex vivo or in vivo. It was demonstrated that pre-coating Onivyde, an FDA-approved NP cancer therapeutic, with human plasma led to enhanced pancreas ductal adenocarcinoma uptake over plasma-free controls. Controls were only incubated in culture media during treatment. Hence, particular protein sources may enrich NP coronas with cancer-honing elements, and in turn, enhance NP uptake in cancer cells. For example, NP incubation in virus-infected sera might enrich NPs with coronal proteins shed during infection, which aid leukocyte extravasation.

The studies herein shows that an increased ability of NPs to cross the endothelium may enhance their tumor access. High drug localization to tumors may ensue. Herein, we study the effects of pre-coating HBPE-NPs with normal or IAV-infected mouse sera on their cancer cell uptake and cancer drug (taxol) delivery efficiency. The biodistribution behavior of PEG-HBPE-NPs and HBPE-NPs that were naturally-coated or IAV sera-coated in TNBC tumor-bearing mice.

The critical interface between the surface of NPs and the surrounding environment is in part defined by the physiochemical characteristics of NPs such as size, charge, or shape that influences the absorption of macromolecules from complex biofluids like blood. When NPs interact with proteins in biofluids, layers form termed the hard or soft coronas. Hard coronas remain relatively stable on NPs in circulation, while soft coronas can undergo a dynamic exchange of their components and are less well understood. Importantly, protein coronas formed on NPs can be specific through the selective binding of proteins from biofluids. For example, studies showed that the ten most abundant proteins found in human blood serum represent ˜86% of the total sera protein content but may only comprise 8-13% of the coronal proteins on NPs. Currently, little is known concerning the intrinsic factors that influence corona development on NPs, nor have the coronal components that contribute to cellular interaction, such as cancer cell uptake, been fully identified. Elucidating these factors is an important step towards enhancing drug delivery mediated by NPs.

Progress in defining the characteristics of coronas formed NPs has mainly depended on the use of normal sera as a source for coronal proteins. The synthesis of polymeric NPs formed can be conducted using hyperbranched polyester (HBPE) polymer that is ideal for the delivery of hydrophobic drug cargo. HBPE-NPs pre-treated with normal mouse sera (NS) displayed enhanced uptake by breast cancer cells that resulted improved delivery of anti-cancer drugs over PEGylated HBPE-NPs. This indicated that proteins present in normal sera were absorbed by the HBPE-NPs that optimized the biological identity of the particles in a manner that improved interactions with cancer cells. One thing that was not achieved with the pre-treatment of HBPE-NPs in normal sera was reduced uptake by monocytes (e.g., THP-1 cells). To enhance the circulation of systemically administered NPs, it is necessary to reduce the uptake by the mononuclear phagocyte system or mononuclear phagocytic system (MPS) also known as the reticuloendothelial system RES) or macrophage system. This is especially important for HBPE-NPs since it was shown that the major portion of these NPs when systemically introduced into a mouse go the liver and spleen where macrophages are abundant. To discover new coronal proteins absorbed by HBPE-NPs that can better concentrate particles in tumors, the use of sera that can contain a more diverse repertoire of proteins was tested.

During the immune response to infection, leukocytes and lymphocytes are activated and traffic through the body as the different stages of the innate and adaptive immune response unfold. In part through the action of proteolytic mechanisms that result in ectodomain shedding, adhesion molecules, cytokines, growth factors, enzymes and others are released into blood. This process is significantly induced when immune cells are activated. Thus, a more complex sera enriched with tissue-targeting proteins could form. To explore whether pre-treating HBPE-NPs with such a sera could enhance cancer targeting, sera from influenza A virus (IAV)-infected mice were used. The IAV infection runs a course from innate to adaptive responses over several days, enabling daily collection of viral infection derived sera (VS) from days 3-6 that contains a diverse collection of shed proteins. Using these sera, HBPE-NPs loaded with either a lipophilic tracking dye or the cancer drug, taxol, were pre-treated and in vitro cell based experiments and in vivo biodistribution studies were performed to characterize the behavior of the HBPE-NPs. It was found that VS5 contained proteins absorbed by the HBPE-NPs that conferred improved tumor cell uptake and reduced monocytic cell uptake. This resulted in accumulation of HBPE-NPs in tumors combined with reduced liver and spleen uptake as compared to PEGylated HBPE-NPs. The most abundant proteins absorbed by HBPE-NPs from VS3 and VS5 were identified, noting differences in the protein profiles relevant to immune response-derived proteins. These findings were confirmed by detecting thrombospondin (TSP-1) as one of the proteins absorbed by NPs. These results support that the protein corona formed on HBPE-NPs is composed of unique proteins essential for cancer cell uptake, validating the presented approach using sera from IAV-infected mice to identify proteins for optimizing the biological identity of tumor-targeted NPs.

Formation of Protein Corona on HBPE-NPs

Research from our lab and others showed that NPs treated with sera from healthy mice (e.g., normal sera) absorbed macromolecules that enhanced tumor cell uptake. Collectively these studies show that the protein corona strongly modulates the biological identity of NPs; hence, defining a protein or groups of proteins that most positively influence the cancer cell uptake of NPs could advance the clinical utility of NPs for drug delivery. To this end, it shows that sera with increased protein content, such as results during an immune response to infection, can be the novel source for discovery of proteins that can be absorbed by NPs and improve the efficiency of drug delivery to tumors.

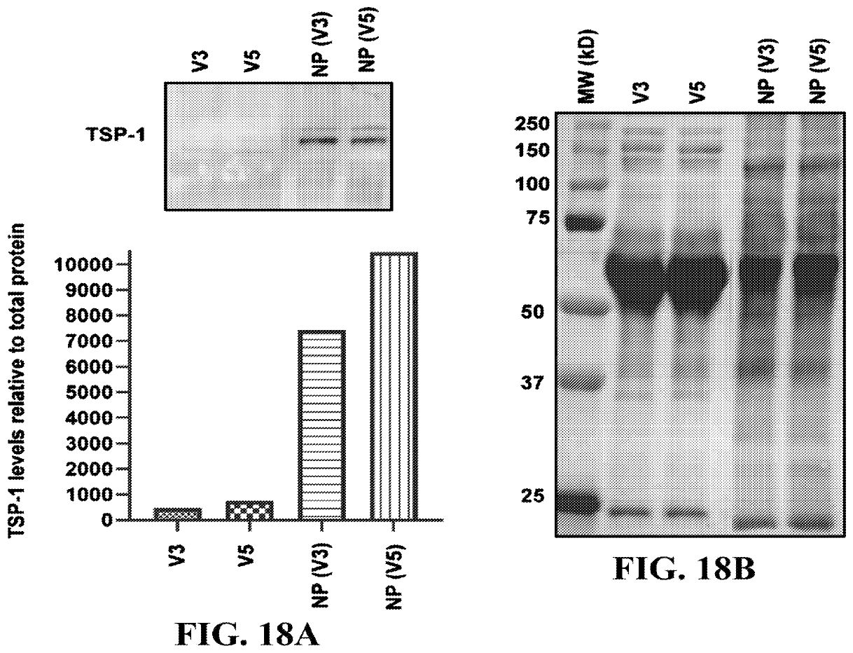

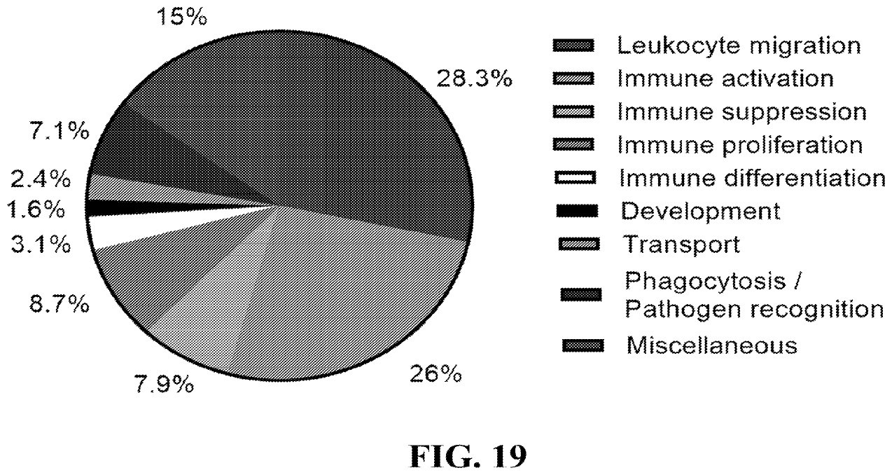

During an infection, proteins are shed from antigen-activated immune cells into the lymph and bloodstream. This is shown in a list of predicted and known shed proteins with immune-related activity generated using DeepSMP (A Deep Learning Model for Predicting the Shedding Events of Membrane Proteins) [csbg-jlu.info/DeepSMP/] and SheddomeDB databases (FIG. 19). The data include immune-related proteins, their probability of shedding (S-score), whether the protein is known to shed via literature support, and the protein's UniProt database ID number, and protein function. It was found that many shed proteins functioned in processes like leukocyte migration and immune cell activation, such stimulating B cells, T cells, and natural killer (NK)-cells. Utilizing sera from infected hosts could thus provide a robust source of coronal proteins for absorption by NPs.

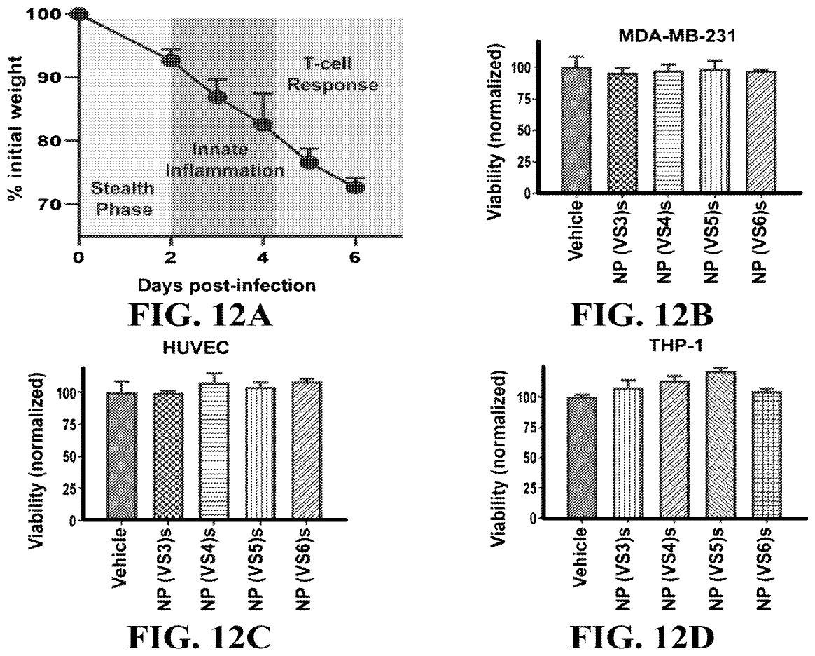

The synthesis of a hyperbranched polyester (HBPE) polymer to form polymeric NPs was optimized. Herein, HBPE-NPs loaded with the tracking dye, DiI or DiR, or the cancer drug, taxol were used, to investigate the characteristics of the protein corona formed when NPs are exposed to sera obtained from mice infected with IAV. PEG-HBPE-NPs were used in all experiments as a standard control. Equal loading of dye or drug in all particles was confirmed. The murine IAV infectious model was chosen since infection proceeds from a stealth phase (days 0-2) with minimal weight loss, to activation of innate (days 2-4) and adaptive (days 4-7) immunity, with significant weight loss and physical deterioration post-7 days (FIG. 12A), enabling evaluation of proteins shed at differences phases of the immune response. Sera were collected from 2-3-month-old female IAV-infected C57BL/6 mice that were inoculated intranasally with a 300 LD50 lethal dose of influenza at days 3-, 4-, 5-, and 6-days post-infection, termed VS3, VS4, VS5 and VS6, and used these sera for pre-forming protein coronas on HBPE-NPs. To form a protein corona on our NPs, HBPE-NPs were incubated in a 20:1 volumetric ratio of NP:sera for 15 min under gentle agitation, as previously established. These conditions were optimized, as increasing, or decreasing the NP:sera ratio, along with altering the incubation time of NPs with sera, changes the protein corona formation and biological outcomes.

To determine whether HBPE-NPs absorbed proteins from VS3-6, the diameter of the HBPE-NPs was analyzed, after incubation with sera, by dynamic light scattering (DLS)1. Briefly, when HBPE-NPs bind sera proteins, such as immunoglobulin G (IgG), anti-IgG-specific antibodies will attach to the immunoglobulin bound to the particles, changing the diameter of the HBPE-NPs, which is measured by DLS. After treatment with VS3-6, HBPE-NPs decreased in average diameter (Table 3); an effect previously noted and likely due to sera proteins constricting the branching of the HBPE polymer. Addition of anti-IgG antibodies lead to increases in the average diameter of HBPE-NPs (VS3), HBPE-NPs (VS4), HBPE-NPs (VS5), and HBPE-NPs (VS6) by ˜21.9%, ˜12.3% ˜6.3%, and ˜11%, respectively. Therefore, HBPE-NPs form a protein corona containing sera proteins produced by IAV-infected mice. While not conclusive, this data suggests that differences detected in the size of NPs could be attributed to the absorption of unique proteins depending on the sera collection day and status of the immune response. This was subsequently confirmed upon identification of sera proteins by mass spectrometry as will be further discussed. Zeta potential was evaluated to determine if the sera from IAV-infected mice altered the surface charge of NPs, which can influence cellular uptake of NPs. Average surface charge of HBPE-NPs (VS3-6) was negative and indicated stability, but on the threshold of tending towards aggregation (Table 3).

TABLE 3
Dynamic Light Scattering Data for HBPE-NPs coated with sera
collected from mice infected with Influenza A virus (IAV)

| | Particle Size | | ζ- potential |
|---|---|---|---|
| Nanoparticle | (nm ± σ) | PDI ± σ | (mV ± σ) |
| COOH-NPs + VS day 3 | 145.9 ± 5.2 | 0.154 ± 0.060 | −24.0 ± 0.9 |
| COOH-NP + VS3 + | 177.9 ± 5.7 | 0.102 ± 0.011 | |
| anti-IgG | | | |
| COOH-NPs + VS day 4 | 148.4 ± 4.4 | 0.183 ± 0.026 | −32.8 ± 1.4 |
| COOH-NPs + VS4 + | 166.6 ± 3.9 | 0.190 ± 0.012 | |
| anti-IgG | | | |
| COOH-NPs + VS day 5 | 147.9 ± 6.8 | 0.179 ± 0.028 | −28.0 ± 2.2 |
| COOH-NPs + VS5 + | 157.3 ± 2.3 | 0.183 ± 0.041 | |
| anti-IgG | | | |
| COOH-NPs + VS day 6 | 143.7 ± 2.0 | 0.174 ± 0.036 | −30.4 ± 1.3 |
| COOH-NPs + VS6 + | 159.5 ± 5.1 | 0.157 ± 0.017 | |
| anti-IgG | | | |

Abbreviations: VS, sera collected from IAV infected mice, numbers refer to the day of sera collection

Having shown that HBPE-NPs form an IAV infection sera-derived protein corona, studies were carried out to rule out any possible cellular toxicities attributed to incorporation of a protein corona derived from VS3-6. Cell lines representing TNBC (MDA-MB-231), endothelial (HUVEC), and monocyte (THP-1) cells were used to assess any potential sera toxicities. MDA-MB-231, HUVEC, or THP-1 cells were incubated with DiI-loaded HBPE-NPs, pre-coated with VS3-6, and, after 24 hours, an MTT assay was performed to assess changes in viability (FIGS. 12B-12D). Results were that pre-treatment of HBPE-NPs with VS3-6 did not induce the toxicity of particles. Cell viability was equivalent to vehicle control.

Direct Uptake HBPE-NPs with Pre-Formed Protein Corona by Monocytic, Endothelial, and Cancer Cells

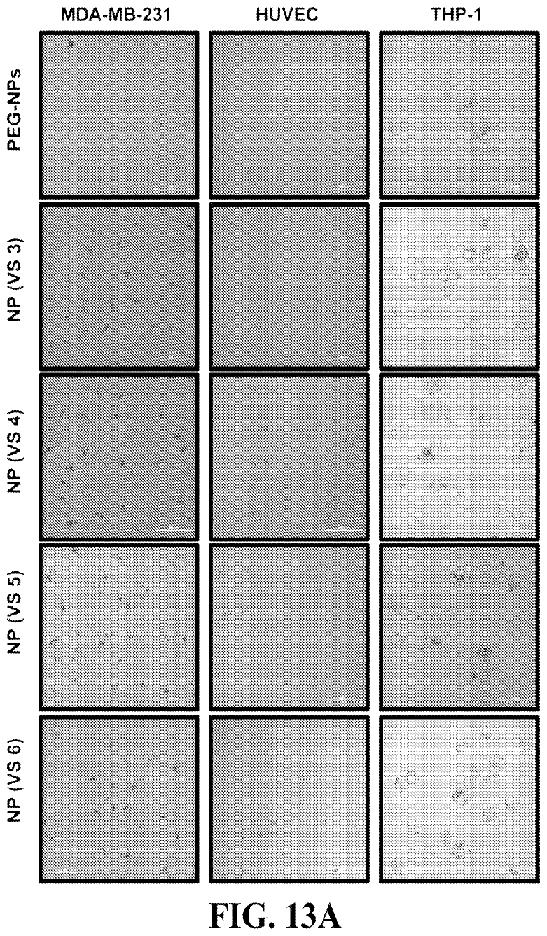

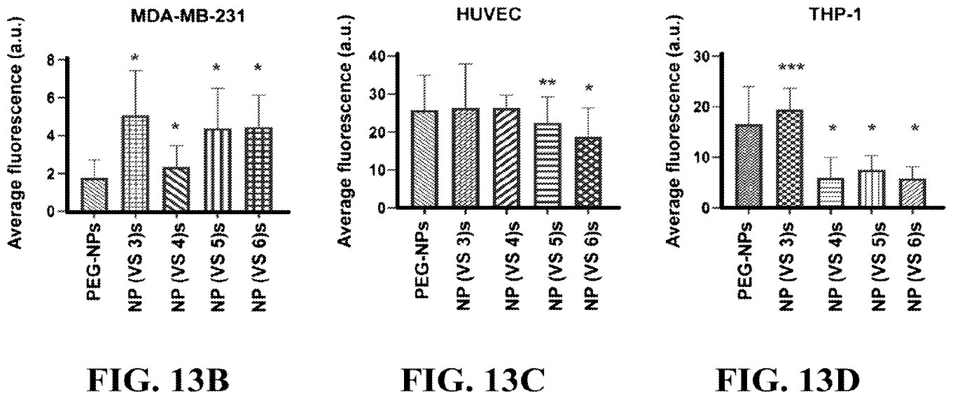

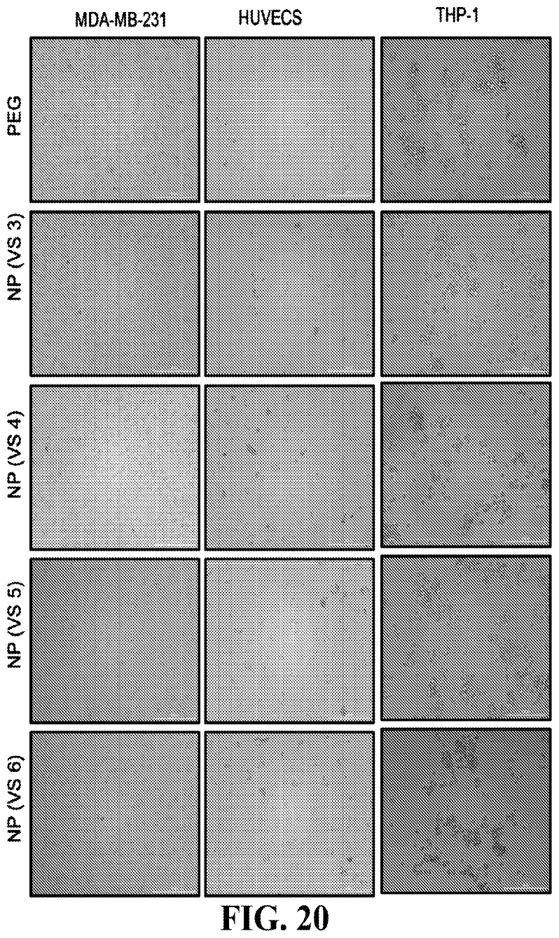

To determine whether pre-coating HBPE-NPs with VS3-6 could modulate cell uptake, MDA-MB-231 (breast cancer), HUVEC (endothelial), and THP-1 (monocytic) cells were treated with VS3-6-coated HBPE-NPs. PEG-HBPE-NPs were used as a standard control. Each formulation of NPs was encapsulated with DiI dye for localization of fluorescent particles in cells. All NPs had comparable concentrations of encapsulated DiI. For cellular imaging of particle uptake, a laser confocal scanning microscope was used for mid-cell plane visualization of NPs. For quantitative analysis of uptake, a multi-modal plate reader with imaging was used for total DiI fluorescence determination. Fluorescence values were calculated by averaging the pixel intensity of individual cells. Cellular uptake of NPs was observed 24 h post-treatment using confocal and digital fluorescent microscopy as described above (FIG. 13A, FIG. 20), and DiI fluorescence per cell was quantified from digital images (FIGS. 13B-13D). In MDA-MB-231 cells, VS3-6-treated HBPE-NPs exhibited a significant (p<0.0001) enhancement in mean uptake or particles as compared to PEG-HBPE-NPs (FIG. 13B). In HUVECs, HBPE-NPs (VS5-treated and V6-treated) and HBPE-NPs (VS6) had reduced average uptake by ˜13.3% (p<0.004) and ˜27.2% (p<0.0001), respectively, relative to PEG-HBPE-NPs (FIG. 13C). In THP-1 cells, HBPE-NPs (VS3-treated) demonstrated a modest mean increase in uptake (p<0.02) by ˜17.4%, while HBPE-NPs (VS4-6 treated) showed a significant reduction in uptake (p<0.0001) by ˜63.9%, ˜54.6%, and ˜64.8%, accordingly, compared to PEG-HBPE-NPs (FIG. 13D). Hence, HBPE-NPs pre-coated with VS4-6 showed enhanced cell delivery to cancer cells over PEG-HBPE-NPs and reduced uptake by monocytes. The modest reduction in uptake in of HBPE-NPs (VS5-6) by HUVECs could be attributed to the transient residence of NPs due to transcellular processes like transcytosis. This study demonstrated that IAV sera-coated HBPE-NPs contain coronal components that improve cancer cell uptake, while reducing immune cell uptake, over PEG modifications.

Drug Delivery to Cancer Cells Using HBPE-NPs with Pre-Formed Protein Corona

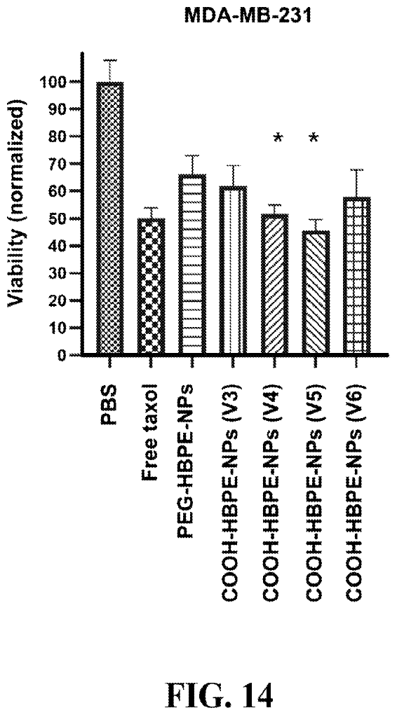

To assess if improved uptake of HBPE-NPs (V3-6) by MDA-MB-231 cells correlated with enhanced delivery of cancer-killing drugs, HBPE-NPs were encapsulated with taxol (paclitaxel). MDA-MB-231 cells were treated with. VS3-6 treated HBPE-NPs and PEG-HBPE-NPs that were loaded with ˜0.5-0.6 μg taxol. After, 24 h post-treatment, cell viability was evaluated using an MTT viability assay (FIG. 14). PBS alone was used as a vehicle control. Free taxol (˜50 nM/43 μg) was used as a positive control. Equivalent taxol loading in NPs was determined. While all NPs delivered toxic doses of taxol, cells incubated with HBPE-NPs (VS4-5-treated) demonstrated a statistically significant reduction in viability as compared to PEG-HBPE-NPs. These results indicate that IAV-sera coated NPs can improve cancer drug delivery over PEGylated NPs. The enhanced toxicity may be attributed to HBPE-NPs (VS4-5) promoting higher uptake in MDA-MB-231 cells over PEG-HBPE-NPs (FIGS. 13B and 20). While the 43 μg dose of free taxol reduced MDA-MB-231 viability by ˜50%, HBPE-NPs (VS4-5 treated), delivering a ˜-80-fold lower dose of taxol (˜0.5 μg), were able to effectively concentrate the lesser amount of drug in cells and decrease MDA-MB-231 viability by an equivalent amount of ˜48% and ˜54%, respectively, as free drug.

Cancer Cell Uptake of HBPE-NPs with Pre-Formed Protein Corona that Move Through an Endothelial Layer

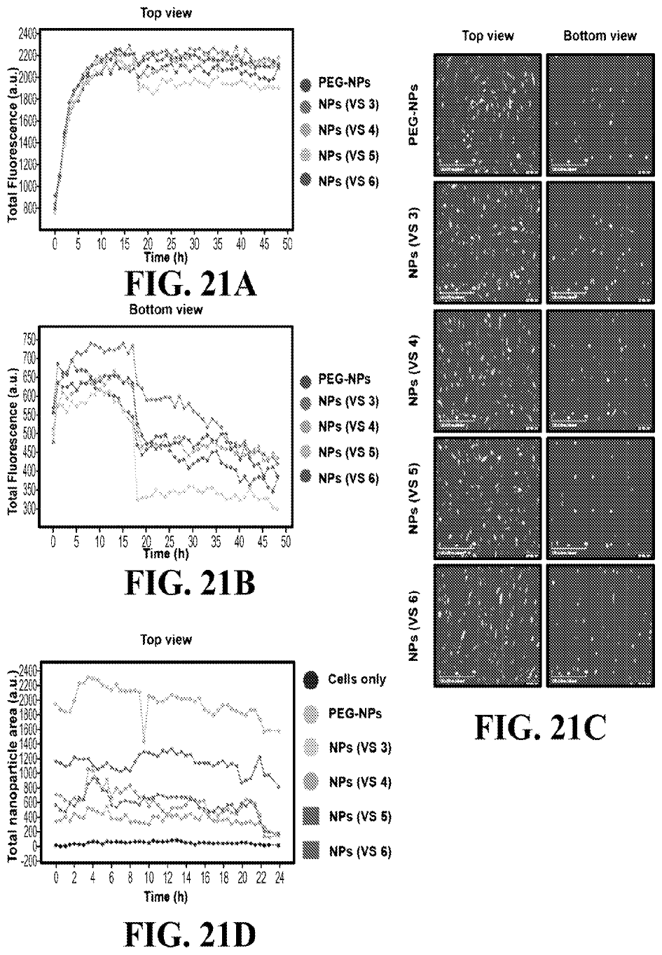

The systemic introduction of drug-loaded NPs into the blood requires NPs to traffic through the endothelial cells that line blood vessels to reach tumors. Studies demonstrated that the ability of a drug to cross an endothelial barrier is improved with nanotechnology-mediated delivery. To determine whether the VS3-5-treated HBPE-NPs can cross an endothelial barrier to be taken up by cancer cells, we used a modified transwell system that mimics aspects of the cellular interactions observed with in vivo delivery conditions. The transwell system features an HUVEC-seeded upper chamber, a porous membrane at the floor of the upper chamber, and an MDA-MB-231-seeded bottom chamber; adopted from. This setup allows in vitro exploration of the movement of NPs to cancer cells through an endothelial layer. Previous work confirmed that HUVEC cells are necessary for the transition of HBPE-NPs from the upper to bottom chamber of the transwell system, since, in the absence of HUVECs, most NPs remain in upper chamber. The confluency of the endothelial layer was optimized at 80% and it was demonstrated that the movement of NPs from upper to lower chamber did not vary with density of the endothelial layer. Importantly, this study confirmed that the interaction of HUVECs with VS3-6-treated HBPE-NPs did not stimulate the migration of HUVECs that could potentially promote angiogenesis (FIG. 21).

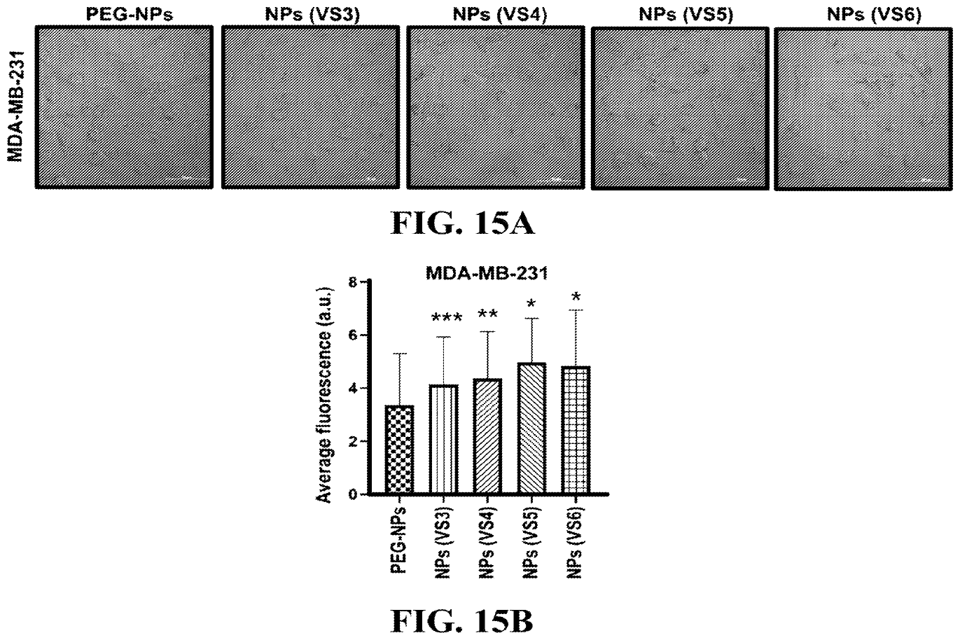

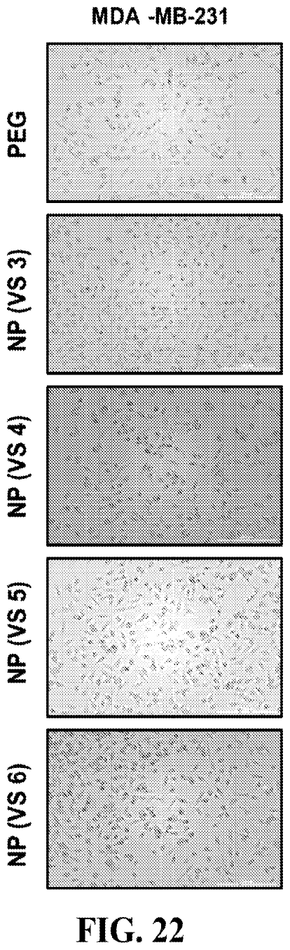

Using the modified transwell system, we ascertained if VS3-6 coated HBPE-NPs loaded with DiI dye can localize to the bottom chamber seeded with cancer cells after initially being dispensed to the upper chamber that is separated from the lower chamber by a membrane and a layer of endothelial cells (HUVECs). PEG-HBPE-NPs were used as a standard control. 0.1 mg HBPE-NPs (VS3-6-treated) or PEG-HBPE-NPs were added to the upper chamber, seeded with HUVECs, and incubated with cells for 24 hours before measuring of uptake of NPs by MDA-MB-231 cells in the bottom chamber, which was visualized with confocal microscopy (FIG. 15A) and quantitated by digital microscopy (FIGS. 15B and 22). Note that the 0.1 mg dose of NPs for the transwell experiments was optimized. Uptake by MDA-MB-231 cells of HBPE-NPs (VS5-treated) and HBPE-NPs (VS6-treated) was significantly higher (p<0.0001) than PEG-HBPE-NPs by ˜48.6% and ˜44.2%, respectively, indicating improved cancer cell uptake of HBPE-NPs, even after prior exposure to endothelial cells. Recall that in the direct uptake studies of MDA-MB-231 cells, we found that HBPE-NPs (VS3-6) demonstrated higher uptake than PEG-HBPE-NPs (FIG. 13B). This did not change in the two-chamber transwell studies in which HBPE-NPs pre-coated with VS3-6, were initially exposed to HUVECs (FIGS. 15A-15B). Hence, the movement of HBPE-NPs, pre-coated with VS3-6, into the bottom chamber to be taken up by cancer cells was not impaired by the initial contact of NPs with endothelial cells and is an area for future optimization to improve the transcytosis of systemically introduced HBPE-NPs.

Biodistribution of HBPE-NPs with Pre-Formed Protein Corona in Mice

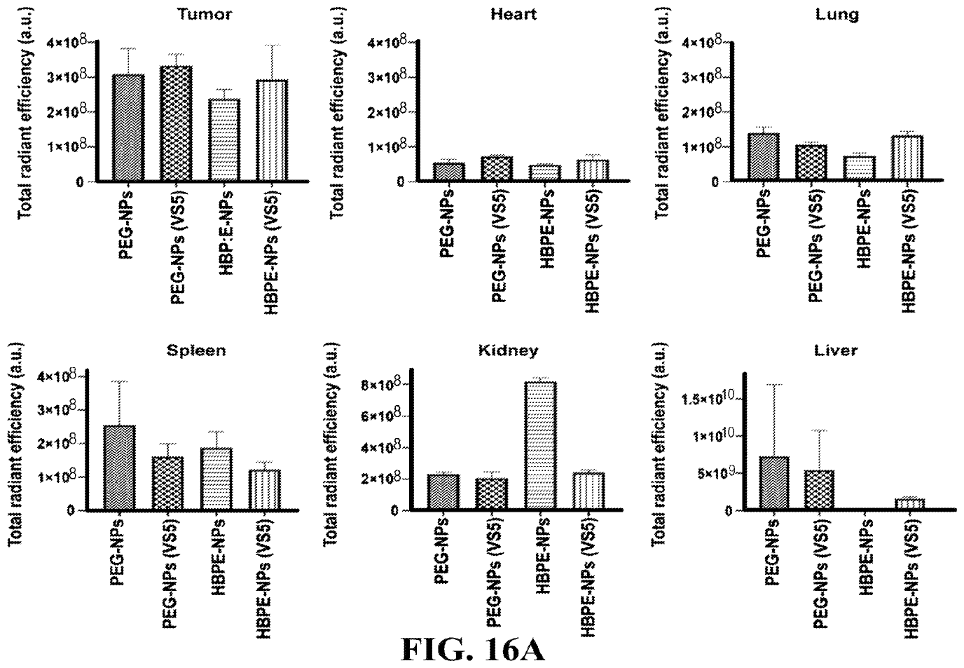

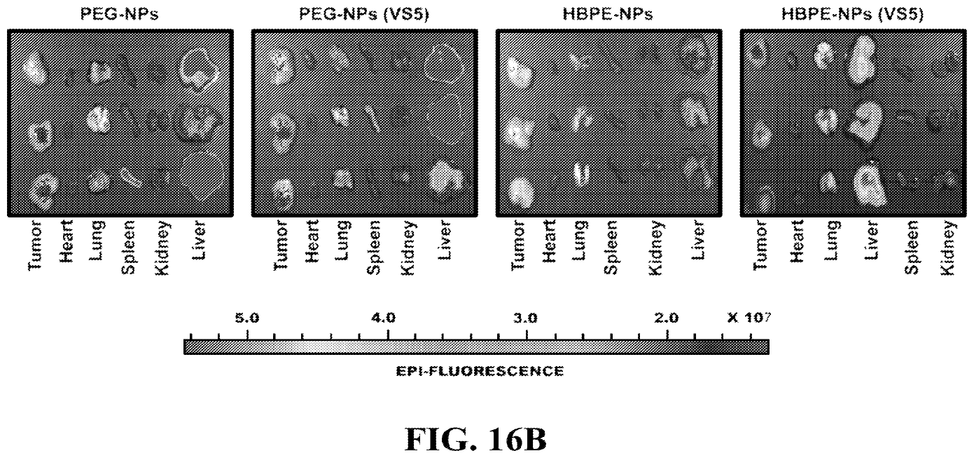

Work to this point indicated that HBPE-NPs treated with VS5 displayed reduced uptake by monocyte-like THP-1 cells (FIG. 13D) and increased uptake by cancer cells (FIGS. 13B, 15B). Uptake by endothelial-like HUVECs was also suggestive a transient interaction (FIG. 13C). For these reasons, the biodistribution of VS5-treated DiR dye-loaded HBPE-NPs was examined in tumor-bearing mice. An orthotopic model of triple negative breast cancer (TNBC) was used in which MDA-MB-231 cells were orthotopically implanted into the mammary fat pad of nude female mice. Once tumors grew to approximately 1000 mm3, DiR infrared dye-loaded HBPE-NPs or PEG-HBPE-NPs were treated with VS5 to pre-form protein coronas and systemically injected into mice through the tail vein. Controls were untreated PEG-HBPE-NPs and HBPE-NPs. After 7 hours, mice were euthanized, and organs were harvested (tumor, heart, lungs, spleen, kidneys, liver) for quantitation of fluorescent signal by imaging. As compared to untreated HBPE-NPs, there was increased tumor uptake of HBPE-NPs (VS5-treated) and this was comparable to PEG-HBPE-NPs (FIGS. 16A-16B). Increased tumor uptake was also noted with PEG-HBPE-NPs when these were pre-treated with VS5 (p=0.0172), indicating that even PEGylated particles were absorbing serum proteins that improved tumor accumulation. Decreased spleen and liver (p=0.0005) uptake of HBPE-NPs pre-treated with VS5 compared to uncoated or PEGylated particles was detected (FIGS. 16A-16B). No significant difference in uptake by the heart was measured, while a slight but statistically significant (p=0.0032 increase in the uptake of VS5-treated HBPE-NPs was seen with the lungs, which is given that the lungs are the major site for the immune response to IAV. Pre-treatment of HBPE-NPs with VS5 also reduced kidney uptake as compared to HBPE-NPs (p<0.001) (FIGS. 16A-16B). From these results, it was concluded that the biodistribution of HBPE-NPs was directly affected by the pre-treated with VS5 since the same core particle (HBPE) and cargo (DiR) were used in the control NPs. Hence, pre-coating with VS5 has potential to confer to NPs the capacity for improved circulation and tumor accumulation.

Identification of Coronal Proteins Absorbed by HBPE-NPs Pre-Treated with VS3 and VS5 sera.

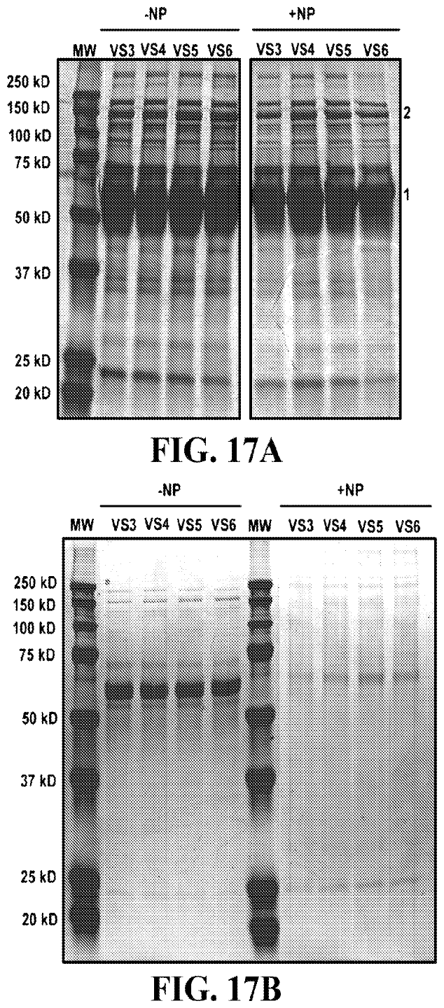

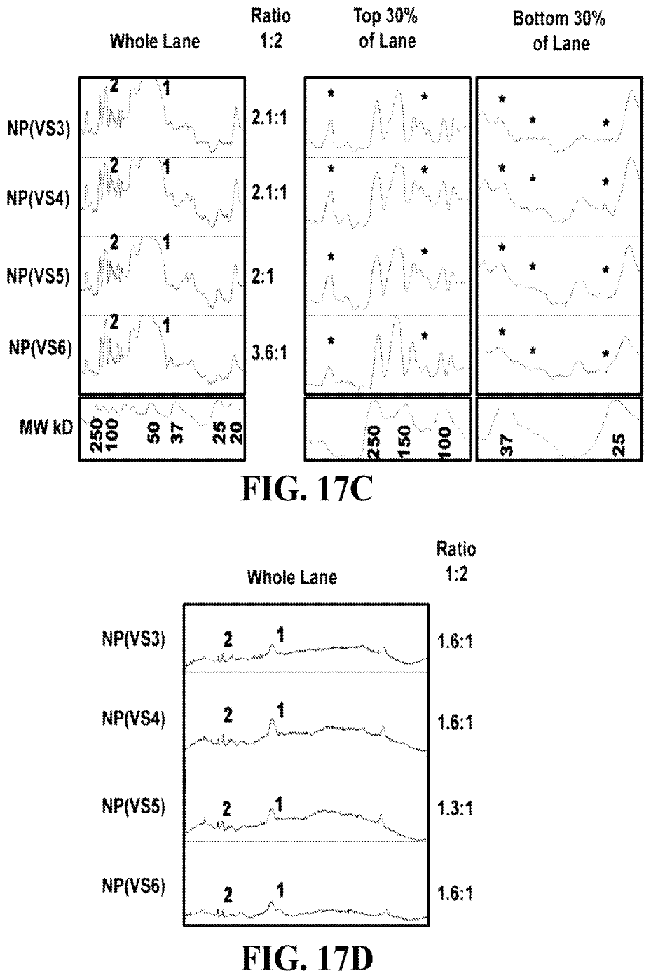

To evaluate the coronal proteins associated with the pre-treatment of NPs with VS3-6, HBPE-NPs were incubated with VS3-6 sera at a 5:1 or 20:1 sera:NP volumetric ratio and proteins absorbed by NPs isolated by high-speed centrifugation. A higher concentrated 5:1 ratio was used to increase protein content for analysis, while the 20:1 ratio reflects the conditions used in the previous experiments. Proteins were analyzed by SDS-PAGE and visualized by Coomassie Blue staining. For the 5:1 sera:NP ratio, the most abundant coronal proteins had molecular weights between 20-25 kD and 50-75 kD, corresponding to major blood proteins like albumin and immunoglobulin (˜60 kDa) (FIGS. 17A, 17B). Differences in the protein patterns in each lane were noted between HBPE-NPs treated with VS3-6 but most noticeably in the top 30% of the high molecular weight proteins (FIGS. 17C-17D). To identify specific proteins found in the protein corona of HBPE-NPs, mass spectrometry analysis of VS3-treated (derived from the innate response to IAV) and VS5-treated (derived from early adaptive immune response to IAV) HBPE-NPs was performed. Both these sera conferred Improved cancer cell uptake but differed in the uptake by monocytes, (FIG. 13). Samples from VS3 and VS5-treated HBPE-NPs were prepared, and controls included VS3 and VS5 alone. Several differences between the proteins absorbed by the HBPE-NPs treated with VS3 vs VS5 were noted.

HBPE-NPs treated with VS3 absorbed more innate immunity-related proteins such as complement and acute phase proteins that can mediate uptake by macrophages, while HBPE-NPs treated VS5 had more proteins involved in coagulation, metabolism, and components of immunoglobulins (Table 4). Functions of identified coronal proteins were assessed via the UniProt database. These results may in part explain the differences in uptake by THP-1 cells (FIG. 13C). A complete list of proteins detected in VS3 and VS5 treated HBPE-NPs as well as sera alone is shown in Table 7. It was further observed that while both VS3 and VS5 contained proteins that can interact with cancer cell receptors, HBPE-NPs treated with VS5 had an abundance of proteins with multi-receptor binding properties, while HBPE-NPs treated with VS3 had more proteins involved in single-receptor/ligand interactions (Table 5). Potential cancer interactions were determined through searching the GEMiCCL (Gene Expression and Mutations in Cancer Cell Lines) database. These findings led the further exploration of the proteins absorbed by HBPE-NPs treated with VS5. As shown in Table 6, HBPE-NPs treated with VS5 were enriched for common sera proteins such as albumin, alpha-2-macroglobulin (pregnancy zone protein), and apoplipoproteins found on other types of NPs. Rarer sera proteins unique to the HBPE-NPs included apolipoprotein A-I, complement factor I, ceruloplasmin and thrombospodin (TSP-1) were also present. While these proteins can be produced by multiple cell types, many of these proteins are also produced by immune cells, such as T lymphocytes. As shown in Table 6, most of these proteins can directly or indirectly promote interactions with MDA-MB-231 cancer cells. These results show that the proteins absorbed VS5 can endow the HBPE-NPs with the ability to better interact with cancer cells. Of these, TSP-1 was of interest since few studies have found it in the protein coronas of other types of polymeric NPs and can interact with multiple cancer receptors.

TABLE 4
Comparison of coronal proteins more abundant in
either VS3- or VS5-treated NPs that could promote
interactions with monocytes/macrophages

| Protein abundance (VS3 > VS5) | Protein abundance (VS5 > VS3) |
|---|---|
| Complement C3 | Albumin |
| Alpha-2-HS-glycoprotein | Fibronectin |
| Complement factor B | Apolipoprotein B-100 |
| Vitronectin | Complement factor H |
| Clusterin | Haptoglobin |
| Inhibitor of carbonic anhydrase | Immunoglobulin heavy constant mu |
| H-2 class I histocompatibility | Complement component C8 beta |
| antigen, Q10 alpha chain | chain |
| Complement C5 | Ig gamma-2B chain C region |
| Carboxypeptidase N subunit 2 | Protein AMBP |
| Plasma protease C1 inhibitor | Ig gamma-1 chain C region, |
| | membrane-bound form |
| Alpha-1-acid glycoprotein 1 | Complement component C8 gamma |
| | chain |
| Alpha-2-antiplasmin | Alpha-1-acid glycoprotein 2 |
| Complement component C8 alpha | Immunoglobulin kappa constant |
| chain | |
| Complement component C9 | Mannose-binding protein C |
| Serum amyloid A-1 protein | Beta-2-microglobulin |
| Complement factor D | Serum amyloid P-component |
| Serum amyloid A-2 protein | Complement C1s-B subcomponent |
| Ig-like domain-containing protein | Transthyretin |
| Complement C1s-A subcomponent | |
| N-acetylmuramoyl-L-alanine | |
| amidase | |
| Carboxypeptidase N catalytic chain | |
| Complement C2 | |
| Complement component 7 | |
| Mannan-binding lectin serine | |
| protease 2 | |
| Ficolin-1 | |
| Complement C1r-A subcomponent | |
| Vitamin K-dependent protein S | |
| Mannan-binding lectin serine | |
| protease 1 | |
| Glyceraldehyde-3-phosphate | |
| dehydrogenase | |
| Vitamin K-dependent protein C | |
| Interleukin-1 receptor accessory | |
| protein | |

TABLE 5
List of top 10 coronal proteins enriched in HBPE-
NPs treated with VS3 or VS5 with potential cancer
interaction (multi-receptors in italics)

| Protein abundance (VS3 > VS5) | | Protein abundance (VS5 > VS3) | |
|---|---|---|---|
| | Potential | | Potential |
| | cancer | | cancer |
| Protein | interaction | Protein | interaction |
| Inter alpha-trypsin | Hyaluronan | Albumin | SPARC, |
| inhibitor, heavy | | | hnRNPs, |
| chain 4 | | | calreticulin, |
| | | | FcRn, Cubilin |
| Alpha-2-HS- | TGF-beta | Pregnancy zone | LRP1, IL-1, |
| glycoprotein | | protein | GRP78 |
| Inter-alpha-trypsin | Hyaluronan | Apolipoprotein | Lipids, lipid |
| inhibitor heavy | | B-100 | receptors |
| chain H2 | | | |
| Clusterin | LDL receptor | Beta-2- | Phospholipids |
| | | glycoprotein 1 | |
| Histidine-rich | Phospholipids | Ceruloplasmin | Albumin, Ctr1, |
| glycoprotein | | | ferritin, |
| | | | ferroportin |
| Afamin | Fatty acids, | Serum | HDL |
| | vitamin E | paraoxonase/ | |
| | receptors | arylesterase 1 | |
| Carboxypeptidase | Kinin receptors | Glutathione | Selenium |
| N subunit 2 | | peroxidase 3 | (ApoER2 + |
| | | | LRP2 + LRP1) |
| Apolipoprotein A- | Lipids, | Insulin-like | IGF-1 (IGF-1 |
| II | lipid receptors | growth factor- | receptors) |
| | | binding protein | |
| | | complex acid | |
| | | labile subunit | |
| Corticosteroid- | Glucocorticoid | Beta-2- | Phospholipids |
| binding globulin | receptors | glycoprotein 1 | |
| Flavin reductase | Riboflavin | Apolipoprotein | Lipids, lipid |
| (NADPH) | receptors | C-III | receptors |

TABLE 6
Compiled list of most abundant coronal proteins on HBPE-NPs
treated with VS5 that had potential cancer interaction

| | | Molecular | Potential cancer |
|---|---|---|---|
| | Accession | weight | interaction |
| Identified protein | # | (kD) | (MDA-MB-231) |
| Albumin | ALBU | 69 | SPARC, hnRNPs, |
| | | | calreticulin, FcRn, |
| | | | Cubilin |
| Alpha-2-HS- | FETUA | 37 | TGF-beta |
| glycoprotein | | | |
| Apolipoprotein A-I | APOA1 | 31 | Lipids, lipid receptors |
| Apolipoprotein A-IV | APOA4 | 45 | Lipids, lipid receptors |
| Apolipoprotein B-100 | APOB | 509 | Lipids, lipid receptors |
| Apolipoprotein E | APOE | 36 | Lipids, lipid receptors |
| Beta-2-glycoprotein 1 | B2MG | 14 | Phospholipids |
| Ceruloplasmin | CERU | 121 | Albumin, Ctr1, |
| | | | ferritin, ferroportin |
| Clusterin | CLUS | 52 | LDL receptor |
| Complement factor I | CFAI | 67 | CD46 (mCRPs) |
| Hemopexin | HEMO | 51 | LRP1 |
| Histidine-rich | APOE | 36 | Phospholipids |
| glycoprotein | | | |
| Inter alpha-trypsin | ITIH4 | 105 | Hyaluronan |
| inhibitor, heavy chain 4 | | | |
| Plasminogen | PLMN | 91 | Apolipoproteins, |
| | | | thrombospondin |
| Pregnancy zone protein | PZP | 166 | LRP1, IL-1, GRP78 |
| Thrombospondin-1 | TSP1 | 130 | Integrins, LRP1, |
| | | | EGFR, TGF-beta, |
| | | | uPA, VEGF-A |

To confirm the identification of proteins absorbed from VS5 by HBPE-NPs, proteins were isolated from HBPE-NPs and performed a western blot for TSP-1. We observed that TSP-1 was more enriched by the NPs as compared to sera alone (FIGS. 18A-18B), confirming the results from the mass spectrometry data (Table 6) and indicating that HBPE-NPs may selectively absorb proteins from sera. Due to its relative size (130 kD) and multiple cancer receptor targeting, TSP-1 can be an important component of the protein corona that is formed on HBPE-NPs and can be incorporated in a rational design for optimizing the tumor accumulation of nanomedicines.

Studies of the protein coronas formed on NPs typically utilize normal sera or plasma from mice or humans. Media containing bovine serum albumin (BSA) and fetal bovine serum (FBS) are also examples. The most common coronal protein from these protein sources is albumin which comprises ˜60% of the blood's protein content. Due to its biological prevalence, biocompatibility, and capacity to target tumor receptors, albumin's use as a cancer therapy has been extensively explored. NPs featuring albumin-enriched coronas have shown promise for use in tumor delivery of drugs. However, albumin-based particles can also exhibit low circulation efficiency in vivo due to rapid immune clearance. Moreover, albumin-based medications can induce severe allergic reactions and other harmful side effects in patients, attributed to poor tissue-specific targeting. Protein coronas on NPs predominantly composed of single ubiquitous sera proteins, like albumin, that closely reflect a serum's native environment cannot be as effective for optimizing tumor accumulation as coronas formed with less abundant proteins that are selectively absorbed by NPs based on biophysical features of the particles.

Using the capacity of NPs to selectively bind or absorb rarer macromolecules from complex biofluids could result in a protein corona that confers a unique biological identity to the particles. For example, it was recently demonstrated that phosphocholine-derived liposomes were capable of passively enriching themselves with shed complement C3 that consequently promoted their localization to pneumonic mouse lungs, through neutrophil trafficking. NPs can also form distinct identities based on their initial exposure to a protein source. When polystyrene NPs were incubated with either human plasma or human cerebrospinal fluid (CSF), coronas comprising different concentrations of the same protein, or unique proteins, formed. For instance, kininogen-1 was the 5th most abundant coronal protein in plasma-incubated NPs, while it was the 15th most abundant in CSF-incubated NPs. Additionally, serum albumin was found only in the plasma-treated NP coronas, when analyzing the top 20 most abundant coronal proteins. These different coronal fingerprints can be heavily influenced by a NP's exposure to individual protein concentration, that varies by the protein source. As an example, analysis of protein coronae from sepsis-affected and non-affected patient plasma-incubated NPs showed that coronal protein concentrations can differ among patient groups by factors of 2 to 317-times. The biggest differences involved immune-related proteins, such as immunoglobulins and complement. Protein content in sera can also change during each day during an IAV infection. A dynamic relationship among protein levels in bronchoalveolar mice lavages at 5-, 14-, and 21-days post-influenza A infection. For example, haptoglobin levels increased 27-, 4-, and 9-fold on days 5, 14, and 21 post-infections compared to pre-infection levels. Hence, the protein coronae derived from VS3-VS6 reflects the variety of macromolecules shed during infection that is be present at the same levels in normal sera and could modify the surface of NPs to favor cancer cell interactions.

Mass spectrometry analysis that we performed using HBPE-NPs identified coronal proteins from VS3-treated and VS5-treated particles that are commonly found with other nanoformulations. These included albumin, complement C3, hemopexin, plasminogen, alpha-2-HS-glycoprotein, and apolipoprotein B-100. Unique proteins to specific nanomaterials have also been discovered. Protein coronas on poly(lactic-co-glycolic acid) (PLGA) and polycaprolactone (PCL) polymeric NPs coated with human sera were compared and common shared proteins were found such as albumin and alpha-2-macroglobulin, but also proteins unique to PCL such as apolipoprotein A-I, cystatin-A, and complement C5. Proteins only associated with PLGA included immunoglobulin kappa variable 3-20. Polystyrene NP coronas have also been examined. Polystyrene NPs incubated in FBS formed coronas containing distinctive proteins like fetuin-B, beta-lactoglobulin, and angiotensinogen among the top 20 most abundant proteins. Proteins rarely found in other polymeric NP corona studies were present in HBPE-NPs examined herein that can have potential cancer binding properties. NPs (VS3-treated) were selectively enriched with inter alpha-trypsin inhibitor heavy chain 4, flavin reductase, and carboxypeptidase N. These proteins can bind to cancer membrane's hyaluronan, riboflavin, and kinin receptors, respectively. NPs (VS5-treated) were selectively enriched with ceruloplasmin, thrombospondin-1, serum paraoxonase, mannose C-binding protein, and Insulin-like growth factor-binding protein complex. These proteins can additionally interface with cancer through copper/iron, integrin, HDL, mannose/fucose, and IGF-1 receptors. The prevalence of cancer-binding coronal proteins can be due to a specialized affinity of proteins to HBPE-NP's unique material. Hence, coating HBPE-NPs with a complex protein source, such as IAV infection-derived sera, can increase the likelihood of enriching coronas with cancer-favoring components.

Another finding was the enhanced uptake of NPs (VS3-treated) by THP-1 cells, while NPs (VS4-VS6-treated) exhibited reduced comparative uptake by THP-1 cells. This phenomenon can be explained by differences in immune-related coronal components between VS3 and VS4-6. The enhanced uptake by THP-1 cells of NPs (VS3-treated) can be in part explained by their coronas being enriched with more monocyte/macrophage-binding proteins compared to NPs (VS5-treated). The greater presence of immune-related proteins, such complement components, on NPs (V3S-treated) can have resulted in a higher overall affinity for THP-1 membranes. Forms of alpha-1-antitrypsin were found more prevalent in NPs (VS3-treated) coronas. Given that alpha-1-antitrypsin has a variety of binding partners that can interact with monocytes, including cytokines, heme, and lipoproteins, they can be an integral factor influencing the corona:THP-1 interface. Another factor contributing to the uptake differences in THP-1s between VS3- and VS5-treated NPs can be differences in their coronal albumin content. Studies have shown that albumin can provide dysopsonin-like properties to NPs. It was demonstrated that pre-coating organosilica NPs with BSA reduced the NPs' uptake in J774A.1 and pMAC cells in vitro compared to NPs not pre-coated with BSA. The authors proposed that BSA can provide NPs with “stealth-like” properties that camouflage them from macrophage detection.

When comparing the analysis of proteins in the coronas of NPs (VS3-treated and VS5-treated), proteins can bind to single or multiple ligand/receptor classes were identified. The enhanced cancer cell uptake of NPs (VS5-treated) can be a result of its corona containing more proteins with multiple cancer-binding properties. Examples of abundant multi cancer-target proteins of NPs (VS5-treated) included pregnancy zone protein, ceruloplasmin, and thrombospondin-1. Ceruloplasmin can directly bind MDA-MB-231s through the copper and iron transporters, Ctrl and ferroportin, respectively. Ceruloplasmin can also indirectly interact with cancer cells through initial serum albumin binding. Thrombospondin-1 (TSP-1) is known to bind integrins, LRP1, EGFR, TGF-beta, uPA, and VEGF-A, which can all associate with cancer. To assess HBPE-NP coronal components that enhance cancer interaction, TSP-1 was chosen as an example multi-target protein for further study. Western blot analysis confirmed that TSP-1 was enriched in NP (VS5-treated). This finding shows that TSP-1 may be an important coronal protein involved in the HBPE-NP: cancer interface that warrants further study.

The pre-treatment of HBPE-NPs with defined sera to produce a rationally designed corona can be advantageous over traditional nanotherapeutics. Coronas can contain a multitude of proteins that interact with cancer membranes in many ways. The formation of a corona on NPs can also reduce inherent NP toxicity, due to NP surfaces being coated with native in vivo macromolecules. Corona coating of silver NPs resulted in reduced inherent toxicity in certain cases. Furthering knowledge of NP corona properties and identifying novel proteins that favor cancer interaction may improve cancer therapeutics. Coronal protein identification can lead to discovering proteins with more effective means of binding cancer cells than conventional approaches targeting cancer receptors like the folate receptor. Moreover, coronal proteins found to be pivotal for tumor delivery may lead to chemically modifying NP surfaces or conventional cancer drugs with molecules that directly bind to cancers or cancer-binding proteins. For instance, physiochemical NP modifications for integrin- and hyaluronan-binding have been studied for cancer targeting. Exploring alternative corona sources from other types of immune infections, such as sepsis or COVID-19, can improve cancer distribution as well. Researching nanomaterials that have high affinity for cancer interactors might additionally result in new therapeutics. Taken together, this study shows a method of improving cancer drug delivery efficiency through a combination of a novel nanomaterial (HBPE) coated with a novel protein corona source (Influenza A-infected sera).

Pretreating HBPE-NPs with sera from mice infected with IAV increased uptake by cancer cells, impacting the biodistribution of HBPE-NPs in vivo. IAV infection-derived sera can contain more proteins that promote cancer cell interactions due to the number of proteins shed during immune cell activation that can also target cancer cell membrane proteins. In MDA-MB-231 breast cancer cells, HBPE-NPs (VS3-VS6 treated) displayed greater uptake over PEG-HBPE-NPs. In THP-1 monocyte cells, HBPE-NPs treated with VS4-VS6 (sera from the adaptive phase of the immune response to IAV) exhibited decreased uptake compared to PEG-HBPE-NPs or HBPE-NPs treated with VS3 (sera from innate part of the immune response) and reduced liver/spleen accumulation. MDA-MB-231 cells treated with paclitaxel (taxol)-loaded HBPE-NPs (VS4-VS5) were more effectively killed with a lower drug dose compared to free drug, and HBPE-NPs (VS3-VS6 sera-coated) actively moved through a HUVEC-seeded layer to be taken up by MDA-MB-231 cells, indicating that the pre-formed coronal layer did not limit the movement of particles through endothelial cells. In mice, HBPE-NPs (VS5-treated) effectively accumulated in tumors compared to untreated HBPE-NPs, while reducing distribution to the spleen and liver compared to PEG-HBPE-NPs. HBPE-NPs (VS5-treated) absorbed a variety of proteins that can improve cancer membrane binding/targeting through interactions with multiple ligands/receptors expressed on cancer cells and had fewer proteins that could interact with myeloid cells. Examples of these included TSP-1 that was highly enriched on VS5-treated HBPE-NPs as compared to VS5 sera alone.

Materials and Methods

Materials: Acetonitrile, potassium bicarbonate (K2CO3), diethyl malonate (DEM), 4-bromobutyl acetate (BBA), iodine, sodium hydroxide (NaOH), and p-toluenesulfonic acid (PTSA) were purchased from MilliporeSigma (Burlington, MA, USA). Ethyl acetate, sodium sulfate (Na2SO4), petroleum ether, methanol, hydrochloric acid (HCl), isopropanol, sodium chloride (NaCl), and dimethyl sulfoxide (DMSO) were obtained from Fisher Scientific (Waltham, MA, USA). Hexadeuterodimethyl sulfoxide (DMSO-d6) and deuterium oxide (D2O) was attained from Acros organics (Geel, Belgium). 1,1′-Dioctadecyl-3,3,3′,3′-tetramethylindocarbocyanine perchlorate (DiI dye) and 1,1′-Dioctadecyl-3,3,3′,3′-tetramethylindotricarbocyanine iodide (DiR dye) were acquired from Life technologies (Carlsbad, CA, USA). Taxol was provided by Cayman chemical (Ann Arbor, MI, USA). 1-ethyl-3-(3-dimethylaminopropyl)carbodiimide (EDC), N-hydroxysuccinimide, and poly(ethylene glycol) 2-aminoethyl ether acetic acid 10,000 MW (PEG) was procured from MilliporeSigma. 2-(N-morpholino)ethanesulfonic acid (MES) 5× buffer (pH 7.4) was bought from Alfa Aesar (Haverhill, MA, USA). Anti-mouse IgG (Fab-specific) goat antibody was purchased from MilliporeSigma. Dulbecco's Modified Eagle Medium (DMEM), L-glutamine, penicillin-streptomycin (10,000 U/mL), endothelial cell growth supplement (ECGS), phosphate-buffered saline (PBS), and 0.25% trypsin (0.1% EDTA in HBSS) were obtained from Corning (Corning, NY, USA). MDA-MB-231 (HTB-26) cells, HUVEC-HUVEC (CRL-1730) cells, THP1 (TIB-202) cells, Ham's F-12K (Kaighn's) medium, and Roswell Park Memorial Institute Medium (RPMI)-1640 were acquired from ATCC (Manassas, VA, USA). Fetal bovine serum (FBS) was provided by Gemini Bio-Products (Sacramento, CA, USA). Heparin sodium salt from porcine intestinal mucosa was procured from MilliporeSigma. 3-(4,5-dimethylthiazolyl-2)-2,5-diphenyl tetrazolium bromide (MTT) was bought from MP Biomedicals (Santa Ana, CA, USA). Neutral buffered formalin (10%) was bought from Azer Scientific Inc. (Morgantown, PA, USA). HUVEC-GFP cells were purchased from Essen BioScience (Goettingen, Germany). MDA-MB-231-GFP cells were acquired from GenTarget Inc. (San Diego, CA, USA). Orange G loading buffer was purchased from BioVision Inc. (Milpitas, CA, USA). β-mercaptoethanol, acetic acid, and ammonium bicarbonate were obtained from MilliporeSigma. Mini-PROTEAN TGX polyacrylamide gels, Precision Plus Protein Dual Color and Standards protein ladder were acquired from Bio-Rad Laboratories (Hercules, CA, USA). Coomassie Brilliant Blue G-250 and fibronectin bovine protein were procured from ThermoFisher Scientific (Waltham, MA, USA). For Western blotting, extra thick blot paper (8.6×13.5 cm) was acquired from Bio-Rad Laboratories. PVDF Immobilon-FL transfer membrane (0.45 μm pore size) were obtained from MilliporeSigma. Ethylenediaminetetraacetic acid (EDTA), glacial acetic acid, glycine, (4-(2-hydroxyethyl)-1-piperazineethanesulfonic acid) (HEPES), Tris(hydroxymethyl)aminomethane (Tris), and Tween20 were purchased from Fisher Scientific. REVERT 700 total protein stain was acquired from Li-Cor Biosciences (Lincoln, NE, USA). Alexa Fluor 790-conjugated thrombospondin-1 (TSP-1) antibody and UltraCruz Blocking Reagent was purchased from Santa Cruz Biotechnology Inc. (Dallas, TX, USA). Protein A/G Plus-Agarose beads were obtained from Santa Cruz Biotechnology Inc.

Nanoparticle Synthesis, Drug Encapsulation, PEG Functionalization, and Encapsulation Quantification

For DiI/DiR dye encapsulation in HBPE-NPs, 10 mg of HBPE polymer was mixed with 1 μg of dye and dissolved at 100 mg/mL in DMSO. To entrap taxol, 10 mg of HBPE polymer was combined with 2 mg of taxol and solubilized in DMSO at 100 mg/mL. Dye or taxol-containing solutions subsequently underwent solvent diffusion for cargo loading. This entailed drop-casting 10 μL of HBPE:dye or HBPE:taxol solution continually into deionized water (4 mL) during vigorous stirring at 2000 rpm, until the entirety of HBPE was dispensed. To conjugate the dye/drug-loaded HBPE-NP surface with PEG, EDC/NHS chemistry was employed. 1.5 mg of EDC dissolved in 100 μL of 1×MES buffer was mixed with HBPE-NPs for 10 s. Afterwards, 0.5 mg of NHS dissolved in 100 μL of 1×MES buffer was incubated with the EDC and HBPE-NP solution for 3 min. 1 mg of PEG was then dissolved in 100 μL of 1×MES buffer was added to the EDC, NHS and HBPE-NP solution for 4 h. The PEG-containing solution was gently rotated with a Rotamix (ATR biotech, Laurel, MD, USA) to enhance PEGylation efficiency. Following HBPE-NP or PEGylated HBPE-NP formation, unreacted chemicals were segregated through a Sephadex G-25 PD-10 desalting column (GE Lifesciences, Chicago, IL, USA). Large particulates were removed through polyethersulfone (PES) membrane (MilliporeSigma) filtration using a 0.22 micron cutoff Purified material was concentrated in deionized water to 10 mg/mL with an Amicon Ultra-4 centrifugal filter unit (Millipore Sigma). NPs were spun at 1,600×g and resuspended every 15 min until the desired concentration was reached. All NP processes were done at 23° C. The cancer drug taxol was encapsulated in NPs and incubated with MDA-MB-231 cells. Methods for quantifying drug loading within NPs. Cells were treated with free taxol (˜50 nM/43 μg) or 0.01 mg of COOH-HBPE-NPs or PEG-HBPE-NPs that were loaded with ˜0.5-0.6 μg taxol. In cell uptake studies, COOH-HBPE-NPs and PEG-HBPE-NPs were loaded with ˜0.8-10 μg DiI.

DLS Analysis and IgG Detection

For size and morphology characterization, a Malvern Zetasizer ZS90 (Malvern Panalytical, Worcestershire, UK) DLS instrument was employed. 10 μL of NPs (10 mg/mL) were mixed with 0.8 mL of deionized water. The diameter and surface charge (zeta potential) of NPs was then determined after the NP solution was added to a folded capillary Zetacell (Malvern Panalytical) cuvette. For analyzing sera-coated NPs, 0.1 mg (20 μL) of NPs was incubated with 1 μL of sera or anti-IgG antibody (2 mg/mL). Sera or antibody immersion with NPs occurred for 15 min, with occasional rotation, prior to DLS analysis. All data measurements were performed in triplicate.

Cell Culture

MDA-MB-231, HUVEC, and THP-1 cells were grown in DMEM, F-12K, or RPMI-1640 culture media, respectively. Media were supplemented with 10% FBS, 1× penicillin-streptomycin, and 2 mM L-glutamine at 37° C. under 5% CO2. F-12K media was additionally enriched with heparin sodium salt (56 mg) and ECGS (15 mg) for HUVEC endothelial cell growth. RPMI-1640 media was enriched with 50 M 2-mercaptoethanol for THP-1 monocyte cell proliferation. Culture media for MDA-MB-231 cells did not require additional nutrients. GFP-expressing MDA-MB-231 and HUVEC cells used identical media to corresponding non-genetically modified cells. Cells were kept at a low passage number.

MTT Viability Assay

To evaluate toxic effects of NP treatment on cells, an MTT viability analysis was performed. Each well of 96-well culture plates featured 0.1 mL of media. All cell types were grown to a 60% density prior to treatment. 0.5×104 of MDA-MB-231, HUVEC, or THP-1 cells were grown at 37° C. under 5% CO2. Wells were treated with 1 or 10 μL of deionized water vehicle, PEG-HBPE-NPs, or HBPE-NPs. NPs were at an initial concentration of 10 mg/mL prior to treatment. Wells were incubated with 50 μg of MTT 24 h post-treatment. Well media was then substituted with 100 μL of DMSO, 4 h after MTT addition. Wells were then agitated at 800 rpm for 15 min. Subsequently, MTT absorbance at 570 nm was read using a Cytation 5 multi-mode reader. Data represent triplicate readings.

Cell Uptake Studies

To evaluate NP distribution in cells, 96-well culture plates (Corning) or 24-well glass bottom culture plates (Cellvis, Mountain View, CA, USA) were employed. 100 μL aliquots of 0.5×104 MDA-MB-231, HUVEC, or THP-1 cells were dispensed in each well. Once cells grew to a 60% density, they were treated with 0.1 mg of non-coated or sera-coated NPs. For sera coating, 20 μL of NP were mixed with 1 μL of sera for 15 min, and were lightly rotated. The 20:1 volumetric ratio of NP-to-sera and NP:sera incubation time of 15 min were found to be optimal conditions for forming the NP corona from previous observations. Cells grew in 5% CO2 atmosphere at 37° C. One day following treatment, well media was initially replaced with PBS. Then, PBS was substituted for 10 min with 10% neutral buffered formalin to fix cells. A final PBS wash followed. NP presence among cells at a mid-plane cellular level, designated by red DiI fluorescence, was imaged via a Zeiss LSM 710 confocal microscope (Carl Zeiss AG, Oberkochen, Germany). Total NP presence within an entire cell was determined through DiI fluorescence imaging by a Cytation 5 multi-mode reader and quantified using ZEN blue software (Carl Zeiss AG). Images and quantification data are representative of three fields of view. 24- and 96-well culture plates were used for confocal and Cytation 5 microscopy, respectively.

Chemotactic Transwell (CT) Protocol

For assessing NP interaction with endothelial cells in real time, GFP-expressing HUVEC (HUVEC-GFP) cells in 60 μL of media were dispensed in a ClearView 96-well chemotaxis plate (Essen BioScience). The chemotaxis plate featured an upper chamber (insert plate) and bottom chamber (reservoir plate) containing HUVEC-GFPs and 200 μL of media alone, respectively. A fibronectin layer was applied over the upper chamber's porous membrane to aid HUVEC-GFP adhesion. Once HUVEC-GFPs, initially seeded at 5×104 cells per well, grew to 80% confluency, they were incubated with 0.1 mg of non-coated or sera-coated NPs. Thereafter, bright-field and green or red fluorescence was imaged with an IncuCyte S3 Live-Cell Analysis System (Essen BioScience) for endothelial cell and NP presence, accordingly. A merged yellow fluorescence indicated green and red co-localization. Time course imaging directly above the upper chamber for 1 or 2 days, at 30 min or 60 min intervals, respectively, was conducted after NP dispension. Chemotaxis software (Essen BioScience) was utilized for fluorescence quantification and time-lapse video creation. Additionally, HUVEC-GFP's ability to migrate toward the reservoir plate was assessed after NP treatment, via tracking HUVEC-GFP above or below the upper chamber's porous membrane, to evaluate the pro-angiogenic potential of NPs. For this, cell culture and treatment methods were identical, as above. HUVEC-GFPs were visualized directly above and beneath the upper chamber's porous membrane, for 48 h at 30 min intervals. GFP signal was measured using the same chemotaxis software over time. All culture plates were incubated in 5% CO2 at room temperature.

Modified Transwell Assay

For single time point analysis of NP uptake behavior in a transwell system, a Millicell-24 cell culture insert plate (MilliporeSigma) was used. This featured an upper chamber seeded with HUVEC cells and a bottom chamber containing media alone. The bottom of the upper chamber comprised of 8 μm pores to permit cell or NP passage. Once the upper chamber, seeded with 3×104 HUVEC cells, became 80% confluent after 48 h, it was transferred atop a 24-well glass-bottom chamber (Cellvis). The bottom chamber was seeded with 5×104 MDA-MB-231 cells and grown to 60% confluency prior to transfer. Culture wells contained 500 μL of media in both chambers. HUVECs were then mixed with 0.1 mg of non-coated or sera-coated NPs. After one day of treatment, in 5% CO2 and 37° C. incubator conditions, MDA-MB-231 cells were PBS washed, formalin-fixed for 15 min, and washed again with PBS. A Zeiss LSM 710 confocal microscope was employed to visualize a single cellular plane of DiI fluorescence in fixed MDA-MB-231s of the bottom chamber, representative of NP transition from the top to bottom chamber. For total DiI fluorescence assessment, a standard 24-well plate (Corning) substituted the glass-bottom chamber for seeding MDA-MB-231s, to allow Cytation 5 imaging. All other culture and treatment conditions remained the same as above. DiI presence was quantified with ZEN blue software.

Mouse Studies

To examine NP biodistribution in vivo, Fox1-nu/nu (nude) female mice were implanted with TNBC cells expressing the luciferase gene (MDA-MB-231-Luc). 8×105 cells were seeded in the mammary fat pad orthotopically. Once the 2-month old female mice possessed tumor volumes of approximately 1000 mm3, NPs were intravenously injected in the tail vein. NPs (1 mg) were PEGylated and encapsulated a DiR near-infrared dye. Organ imaging for DiR localization was performed 7 h post-injection after euthanization. An IVIS Lumina S5 in vivo imaging system (PerkinElmer, Waltham, MA, USA) and Living Image Software (PerkinElmer) was employed for fluorescence visualization and quantification, accordingly. For sera harvesting experiments, 6-9 week old C57BL/6 female mice served as in vivo models. Mice were exposed intranasally to a 50 μL PBS solution containing the H3N2 A/Philippines/2/82/x-79 Influenza A virus at a 300 LD50 lethal dose. Control mice received PBS alone. Viral sera were collected 3, 4, 5, or 6 days post-infection. Terminal cardiac puncture was use for blood acquisition. 1.5 mL Eppendorf tubes (Eppendorf, Hamburg, Germany) were utilized to gather blood. Blood collection tubes were allowed to clot at 37° C. for 60 minutes. Subsequently, clotted blood was spun down for 5 min at 13, 400 rpm using an Eppendorf Minispin (Eppendorf) for sera isolation. Normal or IAV-infected sera was then obtained. Permission for mouse experiments was endorsed by and conducted according to the Institutional Animal Care and Use Committee (IACUC) policies of the University of Central Florida.

Gel Electrophoresis

In order to evaluate the distribution pattern of coronal proteins on NPs, SDS-PAGE gel electrophoresis was performed. NPs were dispensed in sera for 60 min at either 5:1 or 20:1 volumetric NP:sera ratios. Afterwards, to ensure isolation of NP-associated coronal proteins, samples were spun down at 17,000×g for 0.5 h using an Optima TLX ultracentrifuge (Beckman Coulter, Brea, CA, USA) was performed. Following this, cell pellets were washed once in PBS. Washed pellet samples were resuspended in 20 μl of loading buffer. 20 mM β-mercaptoethanol was then dispensed in the loading buffer and samples were heated at 90° C. for 5 minutes to ensure protein dissociation from HBPE-NPs. Samples were then momentarily centrifuged at 16,000×g with a Eppendorf 5415R centrifuge (Eppendorf). 15 μl of pellet samples were pipetted into wells of a Mini-Protean TGX polyacrylamide gel. Gel sample separation was conducted for 150 min at 80 V. A Precision Plus Protein Dual Color Standards protein ladder was selected for molecular weight identification. Samples of diluted mouse sera without NP were also run. Gels were then fixed in a solution of 30% water, 60% methanol, and 10% acetic acid for 1 h, stained with a Coomassie Brilliant Blue G-250 dye for another hour, and destained three times, for 1 h each, with a solution of 60% water, 30% methanol, and 10% acetic acid. Gels were then imaged using an Odyssey infrared imaging system (Li-Cor Biosciences, Lincoln, NE, USA) or ChemiDoc MP imaging system (Bio-Rad Laboratories). Histogram quantification of gel protein bands was created and quantified with ImageJ software.

Mass Spectrometry for Protein Identification

NPs were incubated with IAV day 3 (V3) or 5 (V5) sera for 1 h at a 20:1 volumetric NP:sera ratio. Then, to ensure proteins to be analyzed by mass spectrometry were bound to NPs, samples were centrifuged with a Optima TLX ultracentrifuge (Beckman Coulter, Brea, CA, USA) at 17,000×g for 30 min. Afterwards, cell pellets were washed with ammonium bicarbonate buffer one time. The supernatant of washed samples was removed until 20 μl of solution remained. Uncentrifuged or centrifuged V3 sera- and V5 sera-only controls were used for comparison. Ammonium bicarbonate buffer allowed for trypsin digestion of sample proteins. Samples were then sent to the proteomics division at the University of Florida for coronal protein identification.

Western Blot

To quantify protein presence on NPs a Western Blot was performed. Sera and NP preparation followed the SDS-PAGE methods section above, except for only a 20:1 volumetric NP:sera ratio was used. After SDS-PAGE was run, the gel was rinsed in deionized water and placed in transfer buffer for 10 minutes under gentle shaking. Transfer buffer was prepared as follows: 1.9 M glycine, 250 mM Tris (Tris(hydroxymethyl)aminomethane), 1 mM EDTA—diluted by 10 times in 20% methanol and deionized water. Then, a PVDF membrane was activated in methanol for 1 min and afterwards placed in transfer buffer for 10 minutes under gentle shaking. Following this, a gel “sandwich” was assembled consisting of filter paper, the activated PVDF membrane, the gel and filter paper in bottom-to-top order. The gel sandwich was assembled on a Bio-Rad Trans-Blot SD Semi-Dry transfer cell and the transfer was run for 60 min at 20 V using a Bio-Rad PowerPac HC. After transfer, the gel was discarded and the membrane was stained for 5 min with 5 mL of REVERT 700 total protein stain under gentle shaking. The membrane was then washed twice with 5 mL of REVERT wash solution for 30 s each time. REVERT wash solution was prepared as follows: 6.7% (v/v) glacial acetic acid and 30% (v/v) methanol in deionized water. Subsequently, the gel was rinsed in deionized water and total protein was imaged using a Li-Cor Odyssey imaging system at a 700 nm wavelength. REVERT stain was afterwards removed with 5 mL of REVERT reversal solution for 5 min. REVERT reversal solution was prepared as follows: 0.1 M sodium hydroxide and 30% (v/v) methanol in deionized water. The membrane was then incubated in 5 mL of UltraCruz Blocking Reagent blocking buffer for 60 min under gentle shaking. Following this, the membrane was incubated in 5 mL of blocking buffer and 5 mL of TSP-1 antibody overnight at 4° C. under gentle shaking. For antibody incubation, the antibody was diluted 1:2000 in HBST solution. HBST solution was prepared as follows: 100 mM HEPES (pH 7.5), 750 mM NaCl, and 1 mM EDTA—diluted by 5 times in deionized water and 0.05% Tween20. After overnight incubation, the membrane was rinsed 3 times, 5 min each, in 5 mL of HBST solution. Membranes were then visualized at a 800 nm wavelength for TSP-1 antibody presence using a Li-Cor Odyssey imaging system.

Statistical Analysis

Graphpad Prism 8 software was used to distinguish statistical significance. Analysis was done with Welch's correction of a T test applied to parametric, unpaired, and two-tailed conditions. This allowed statistical assessment between two experimental groups of data. P-values <0.05, representing a 95% confidence level are designated in figure legends and were deemed statistically significant.

TABLE 7

| | | | | V5 Sera Alone | V5 Sera_Nano- |
|---|---|---|---|---|---|
| | | | | (Control) | particles |
| | | | | Quantitative | Quantitative |
| | | | | Value | Value |
| | Accession | Molecular | | (Normalized | (Normalized |
| Identified Proteins (350) | Number | Weight | | Total Spectra) | Total Spectra) |
| Albumin OS = Mus musculus (Mouse) | ALBU_MOUSE | 69 | kDa | 612.5 | 1,009.60 |
| OX = 10090 GN = Alb PE = 1 SV = 3 | | | | | |
| Complement C3 OS = Mus musculus | CO3_MOUSE | 186 | kDa | 161.91 | 159.92 |
| (Mouse) OX = 10090 GN = C3 PE = 1 SV = 3 | | | | | |
| Pregnancy zone protein OS = Mus | PZP_MOUSE | 166 | kDa | 112.88 | 144.69 |
| musculus (Mouse) OX = 10090 GN = Pzp | | | | | |
| PE = 1 SV = 3 | | | | | |
| Apolipoprotein A-I OS = Mus musculus | APOA1_MOUSE | 31 | kDa | 112.88 | 102.26 |
| (Mouse) OX = 10090 GN = Apoa1 PE = 1 | | | | | |
| SV = 2 | | | | | |
| Cluster of GLOBIN domain-containing | A8DUK4_MOUSE | 16 | kDa | 111.95 | 100.09 |
| protein OS = Mus musculus (Mouse) | [2] | | | | |
| OX = 10090 GN = Hbb-bs PE = 1 SV = 1 | | | | | |
| (A8DUK4_MOUSE) | | | | | |
| GLOBIN domain-containing protein | A8DUK4_MOUSE | 16 | kDa | 111.95 | 99.001 |
| OS = Mus musculus (Mouse) OX = 10090 | | | | | |
| GN = Hbb-bs PE = 1 SV = 1 | | | | | |
| Serotransferrin OS = Mus musculus | TRFE_MOUSE | 77 | kDa | 136.93 | 96.825 |
| (Mouse) OX = 10090 GN = Tf PE = 1 SV = 1 | | | | | |
| Ceruloplasmin OS = Mus musculus | CERU_MOUSE | 121 | kDa | 45.336 | 87.034 |
| (Mouse) OX = 10090 GN = Cp PE = 1 SV = 2 | | | | | |
| Cluster of Murinoglobulin-1 OS = Mus | MUG1_MOUSE | 165 | kDa | 61.99 | 63.099 |
| musculus (Mouse) OX = 10090 GN = Mug1 | [2] | | | | |
| PE = 1 SV = 3 (MUG1_MOUSE) | | | | | |
| Murinoglobulin-1 OS = Mus musculus | MUG1_MOUSE | 165 | kDa | 61.99 | 63.099 |
| (Mouse) OX = 10090 GN = Mug1 PE =1 | | | | | |
| SV = 3 | | | | | |
| Hemopexin OS = Mus musculus (Mouse) | HEMO_MOUSE | 51 | kDa | 53.663 | 62.011 |
| OX = 10090 GN = Hpx PE = 1 SV = 2 | | | | | |
| Cluster of Serine protease inhibitor A3K | SPA3K_MOUSE | 47 | kDa | 60.14 | 62.011 |
| OS = Mus musculus (Mouse) OX = 10090 | [4] | | | | |
| GN = Serpina3k PE = 1 SV = 2 | | | | | |
| (SPA3K_MOUSE) | | | | | |
| Serine protease inhibitor A3K | SPA3K_MOUSE | 47 | kDa | 53.663 | 56.572 |
| OS = Mus musculus (Mouse) OX = 10090 | | | | | |
| GN = Serpina3k PE = 1 SV = 2 | | | | | |
| Kininogen-1 OS = Mus musculus (Mouse) | KNG1_MOUSE | 73 | kDa | 44.411 | 56.572 |
| OX = 10090 GN = Kng1 PE = 1 SV = 1 | | | | | |
| Hemoglobin subunit beta-2 OS = Mus | HBB2_MOUSE | 16 | kDa | 24.981 | 54.396 |
| musculus (Mouse) OX = 10090 GN = Hbb- | | | | | |
| b2 PE = 1 SV = 2 | | | | | |
| Plasminogen OS = Mus musculus (Mouse) | PLMN_MOUSE | 91 | kDa | 40.71 | 53.308 |
| OX = 10090 GN = Plg PE = 1 SV = 3 | | | | | |
| Haptoglobin OS = Mus musculus (Mouse) | HPT_MOUSE | 39 | kDa | 47.187 | 53.308 |
| OX = 10090 GN = Hp PE = 1 SV = 1 | | | | | |
| Cluster of Alpha-1-antitrypsin 1-4 | A1AT4_MOUSE | 46 | kDa | 78.644 | 51.132 |
| OS = Mus musculus (Mouse) OX = 10090 | [4] | | | | |
| GN = Serpina1d PE = 1 SV = 1 | | | | | |
| (A1AT4_MOUSE) | | | | | |
| Alpha-2-HS-glycoprotein OS = Mus | FETUA_MOUSE | 37 | kDa | 32.383 | 46.781 |
| musculus (Mouse) OX = 10090 GN = Ahsg | | | | | |
| PE = 1 SV = 1 | | | | | |
| Thrombospondin-1 OS = Mus musculus | TSP1_MOUSE | 130 | kDa | 24.981 | 44.605 |
| (Mouse) OX = 10090 GN = Thbs1 PE = 1 | | | | | |
| SV = 1 | | | | | |
| Hemoglobin subunit alpha OS = Mus | HBA_MOUSE | 15 | kDa | 28.682 | 41.341 |
| musculus (Mouse) OX = 10090 GN = Hba | | | | | |
| PE = 1 SV = 2 | | | | | |
| Alpha-1-antitrypsin 1-4 OS = Mus | A1AT4_MOUSE | 46 | kDa | 60.14 | 40.253 |
| musculus (Mouse) OX = 10090 | | | | | |
| GN = Serpina1d PE = 1 SV = 1 | | | | | |
| Murinoglobulin-2 OS = Mus musculus | MUG2_MOUSE | 162 | kDa | 29.607 | 39.165 |
| (Mouse) OX = 10090 GN = Mug2 PE = 1 | | | | | |
| SV = 2 | | | | | |
| Alpha-1-antitrypsin 1-1 OS = Mus | A1AT1_MOUSE | 46 | kDa | 55.514 | 35.901 |
| musculus (Mouse) OX = 10090 | | | | | |
| GN = Serpina1a PE = 1 SV = 4 | | | | | |
| Alpha-1-antitrypsin 1-2 OS = Mus | A1AT2_MOUSE | 46 | kDa | 58.289 | 35.901 |
| musculus (Mouse) OX = 10090 | | | | | |
| GN = Serpina1b PE = 1 SV = 2 | | | | | |
| Inter alpha-trypsin inhibitor, heavy | ITIH4_MOUSE | 105 | kDa | 70.317 | 35.901 |
| chain 4 OS = Mus musculus (Mouse) | | | | | |
| OX = 10090 GN = Itih4 PE = 1 SV = 2 | | | | | |
| Fibronectin OS = Mus musculus (Mouse) | FINC_MOUSE | 273 | kDa | 33.308 | 35.901 |
| OX = 10090 GN = Fn1 PE = 1 SV = 4 | | | | | |
| Vitamin D-binding protein OS = Mus | VTDB_MOUSE | 54 | kDa | 36.084 | 33.726 |
| musculus (Mouse) OX = 10090 GN = Gc | | | | | |
| PE = 1 SV = 2 | | | | | |
| Prothrombin OS = Mus musculus (Mouse) | THRB_MOUSE | 70 | kDa | 63.841 | 31.55 |
| OX = 10090 GN = F2 PE = 1 SV = 1 | | | | | |
| Spectrin beta chain, erythrocytic | SPTB1_MOUSE | 245 | kDa | 3.7009 | 31.55 |
| OS = Mus musculus (Mouse) OX = 10090 | | | | | |
| GN = Sptb PE = 1 SV = 4 | | | | | |
| Serine protease inhibitor A3M | SPA3M_MOUSE | 47 | kDa | 27.757 | 28.286 |
| OS = Mus musculus (Mouse) OX = 10090 | | | | | |
| GN = Serpina3m PE = 1 SV = 2 | | | | | |
| Cluster of Complement factor H | CFAH_MOUSE | 139 | kDa | 125.83 | 27.198 |
| OS = Mus musculus (Mouse) OX = 10090 | [2] | | | | |
| GN = Cfh PE = 1 SV = 2 (CFAH_MOUSE) | | | | | |
| Complement factor H OS = Mus | CFAH_MOUSE | 139 | kDa | 124.91 | 27.198 |
| musculus (Mouse) OX = 10090 GN = Cfh | | | | | |
| PE = 1 SV = 2 | | | | | |
| Apolipoprotein E OS = Mus musculus | APOE_MOUSE | 36 | kDa | 63.841 | 26.11 |
| (Mouse) OX = 10090 GN = Apoe PE = 1 | | | | | |
| SV = 2 | | | | | |
| Apolipoprotein A-IV OS = Mus musculus | APOA4_MOUSE | 45 | kDa | 57.364 | 25.022 |
| (Mouse) OX = 10090 GN = Apoa4 PE = 1 | | | | | |
| SV = 3 | | | | | |
| Cluster of Carboxylesterase 1C OS = Mus | EST1C_MOUSE | 61 | kDa | 30.533 | 25.022 |
| musculus (Mouse) OX = 10090 GN = Ces1c | [3] | | | | |
| PE = 1 SV = 4 (EST1C_MOUSE) | | | | | |
| Complement factor B OS = Mus musculus | CFAB_MOUSE | 85 | kDa | 22.205 | 25.022 |
| (Mouse) OX = 10090 GN = Cfb PE = 1 SV = 2 | | | | | |
| Beta-2-glycoprotein 1 OS = Mus musculus | APOH_MOUSE | 39 | kDa | 21.28 | 25.022 |
| (Mouse) OX = 10090 GN = Apoh PE = 1 | | | | | |
| SV = 1 | | | | | |
| Cluster of Ankyrin-1 OS = Mus musculus | ANK1_MOUSE | 204 | kDa | 2.7757 | 25.022 |
| (Mouse) OX = 10090 GN = Ank1 PE = 1 | [3] | | | | |
| SV = 2 (ANK1_MOUSE) | | | | | |
| Ankyrin-1 OS = Mus musculus (Mouse) | ANK1_MOUSE | 204 | kDa | 2.7757 | 25.022 |
| OX = 10090 GN = Ank1 PE = 1 SV = 2 | | | | | |
| Spectrin alpha chain, erythrocytic 1 | SPTA1_MOUSE | 280 | kDa | 2.7757 | 22.846 |
| OS = Mus musculus (Mouse) OX = 10090 | | | | | |
| GN = Spta1 PE = 1 SV = 3 | | | | | |
| Carboxylesterase 1C OS = Mus | EST1C_MOUSE | 61 | kDa | 28.682 | 21.758 |
| musculus (Mouse) OX = 10090 GN = Ces1c | | | | | |
| PE = 1 SV = 4 | | | | | |
| Apolipoprotein B-100 OS = Mus musculus | APOB_MOUSE | 509 | kDa | 19.43 | 21.758 |
| (Mouse) OX = 10090 GN = Apob PE = 1 | | | | | |
| SV = 1 | | | | | |
| Serine protease inhibitor A3N | SPA3N_MOUSE | 47 | kDa | 19.43 | 20.67 |
| OS = Mus musculus (Mouse) OX = 10090 | | | | | |
| GN = Serpina3n PE = 1 SV = 1 | | | | | |
| Inhibitor of carbonic anhydrase OS = Mus | ICA_MOUSE | 77 | kDa | 9.2523 | 20.67 |
| musculus (Mouse) OX = 10090 GN = Ica | | | | | |
| PE = 1 SV = 1 | | | | | |
| Complement factor I OS = Mus musculus | CFAI_MOUSE | 67 | kDa | 17.579 | 19.583 |
| (Mouse) OX = 10090 GN = Cfi PE = 1 SV = 3 | | | | | |
| Complement C4-B OS = Mus musculus | CO4B_MOUSE | 193 | kDa | 36.084 | 18.495 |
| (Mouse) OX = 10090 GN = C4b PE = 1 SV = 3 | | | | | |
| Histidine-rich glycoprotein OS = Mus | HRG_MOUSE | 59 | kDa | 14.804 | 18.495 |
| musculus (Mouse) OX = 10090 GN = Hrg | | | | | |
| PE = 1 SV = 2 | | | | | |
| Cluster of Actin, cytoplasmic 1 OS = Mus | ACTB_MOUSE | 42 | kDa | 12.953 | 18.495 |
| musculus (Mouse) OX = 10090 GN = Actb | [4] | | | | |
| PE = 1 SV = 1 (ACTB_MOUSE) | | | | | |
| Actin, cytoplasmic 1 OS = Mus | ACTB_MOUSE | 42 | kDa | 12.028 | 17.407 |
| musculus (Mouse) OX = 10090 GN = Actb | | | | | |
| PE = 1 SV = 1 | | | | | |
| Afamin OS = Mus musculus (Mouse) | AFAM_MOUSE | 69 | kDa | 12.028 | 17.407 |
| OX = 10090 GN = Afm PE = 1 SV = 2 | | | | | |
| Inter-alpha-trypsin inhibitor heavy chain | ITIH2_MOUSE | 106 | kDa | 14.804 | 17.407 |
| H2 OS = Mus musculus (Mouse) | | | | | |
| OX = 10090 GN = Itih2 PE = 1 SV = 1 | | | | | |
| Protein AMBP OS = Mus musculus | AMBP_MOUSE | 39 | kDa | 10.178 | 17.407 |
| (Mouse) OX = 10090 GN = Ambp PE = 1 | | | | | |
| SV = 2 | | | | | |
| Actin, alpha cardiac muscle 1 OS = Mus | ACTC_MOUSE | 42 | kDa | 8.327 | 16.319 |
| musculus (Mouse) OX = 10090 GN = Actc1 | (+1) | | | | |
| PE = 1 SV = 1 | | | | | |
| Inter-alpha-trypsin inhibitor heavy chain | ITIH3_MOUSE | 99 | kDa | 11.103 | 16.319 |
| H3 OS = Mus musculus (Mouse) | | | | | |
| OX = 10090 GN = Itih3 PE = 1 SV = 3 | | | | | |
| LRRCT domain-containing protein | Q91XL1_MOUSE | 37 | kDa | 9.2523 | 16.319 |
| OS = Mus musculus (Mouse) OX = 10090 | | | | | |
| GN = Lrg1 PE = 1 SV = 1 | | | | | |
| Vitronectin OS = Mus musculus (Mouse) | VTNC_MOUSE | 55 | kDa | 59.215 | 15.231 |
| OX = 10090 GN = Vtn PE = 1 SV = 2 | | | | | |
| Clusterin OS = Mus musculus (Mouse) | CLUS_MOUSE | 52 | kDa | 37.934 | 15.231 |
| OX = 10090 GN = Clu PE = 1 SV = 1 | | | | | |
| Phosphatidylinositol-glycan-specific | PHLD_MOUSE | 93 | kDa | 25.906 | 15.231 |
| phospholipase D OS = Mus musculus | | | | | |
| (Mouse) OX = 10090 GN = Gpld1 PE = 1 | | | | | |
| SV = 1 | | | | | |
| Plasma kallikrein OS = Mus musculus | KLKB1_MOUSE | 71 | kDa | 14.804 | 15.231 |
| (Mouse) OX = 10090 GN = Klkb1 PE = 1 | | | | | |
| SV = 2 | | | | | |
| Complement C5 OS = Mus musculus | CO5_MOUSE | 189 | kDa | 18.505 | 14.143 |
| (Mouse) OX = 10090 GN = C5 PE = 1 SV = 2 | | | | | |
| Cluster of Keratin, type II cytoskeletal 2 | K22E_MOUSE | 71 | kDa | 36.084 | 13.055 |
| epidermal OS = Mus musculus (Mouse) | [13] | | | | |
| OX = 10090 GN = Krt2 PE = 1 SV = 1 | | | | | |
| (K22E_MOUSE) | | | | | |
| Cluster of H-2 class I histocompatibility | HA10_MOUSE | 37 | kDa | 14.804 | 13.055 |
| antigen, Q10 alpha chain OS = Mus | [5] | | | | |
| musculus (Mouse) OX = 10090 GN = H2- | | | | | |
| Q10 PE = 1 SV = 3 (HA10_MOUSE) | | | | | |
| H-2 class I histocompatibility antigen, | HA10_MOUSE | 37 | kDa | 13.878 | 13.055 |
| Q10 alpha chain OS = Mus musculus | | | | | |
| (Mouse) OX = 10090 GN = H2-Q10 PE = 1 | | | | | |
| SV = 3 | | | | | |
| Band 3 anion transport protein OS = Mus | B3AT_MOUSE | 103 | kDa | 2.7757 | 13.055 |
| musculus (Mouse) OX = 10090 GN = Slc4a1 | | | | | |
| PE = 1 SV = 1 | | | | | |
| Carboxylic ester hydrolase OS = Mus | D3Z5G7_MOUSE | 62 | kDa | 12.028 | 11.967 |
| musculus (Mouse) OX = 10090 GN = Ces1b | | | | | |
| PE = 1 SV = 1 | | | | | |
| Immunoglobulin heavy constant mu | IGHM_MOUSE | 50 | kDa | 23.131 | 11.967 |
| OS = Mus musculus (Mouse) OX = 10090 | | | | | |
| GN = Ighm PE = 1 SV = 2 | | | | | |
| Inter-alpha-trypsin inhibitor heavy chain | ITIH1_MOUSE | 101 | kDa | 12.953 | 10.879 |
| H1 OS = Mus musculus (Mouse) | | | | | |
| OX = 10090 GN = Itih1 PE = 1 SV = 2 | | | | | |
| Serum amyloid P-component OS = Mus | SAMP_MOUSE | 26 | kDa | 4.6261 | 10.879 |
| musculus (Mouse) OX = 10090 GN = Apcs | | | | | |
| PE = 1 SV = 2 | | | | | |
| Apolipoprotein M OS = Mus musculus | APOM_MOUSE | 21 | kDa | 6.4766 | 10.879 |
| (Mouse) OX = 10090 GN = Apom PE = 1 | | | | | |
| SV = 1 | | | | | |
| Beta-actin-like protein 2 OS = Mus | ACTBL_MOUSE | 42 | kDa | 4.6261 | 9.7913 |
| musculus (Mouse) OX = 10090 GN = Actbl2 | | | | | |
| PE = 1 SV = 1 | | | | | |
| Gelsolin OS = Mus musculus (Mouse) | GELS_MOUSE | 86 | kDa | 16.654 | 9.7913 |
| OX = 10090 GN = Gsn PE = 1 SV = 3 | | | | | |
| Antithrombin-III OS = Mus musculus | ANT3_MOUSE | 52 | kDa | 11.103 | 9.7913 |
| (Mouse) OX = 10090 GN = Serpinc1 PE = 1 | | | | | |
| SV = 1 | | | | | |
| Alpha-1B-glycoprotein OS = Mus | A1BG_MOUSE | 57 | kDa | 6.4766 | 9.7913 |
| musculus (Mouse) OX = 10090 GN = A1bg | | | | | |
| PE = 1 SV = 1 | | | | | |
| CD5 antigen-like OS = Mus musculus | CD5L_MOUSE | 39 | kDa | 9.2523 | 9.7913 |
| (Mouse) OX = 10090 GN = Cd5l PE = 1 | | | | | |
| SV = 3 | | | | | |
| Sulfhydryl oxidase 1 OS = Mus musculus | QSOX1_MOUSE | 83 | kDa | 6.4766 | 9.7913 |
| (Mouse) OX = 10090 GN = Qsox1 PE = 1 | | | | | |
| SV = 1 | | | | | |
| HMW kininogen-II OS = Mus musculus | Q6S9I3_MOUSE | 71 | kDa | 9.2523 | 9.7913 |
| (Mouse) OX = 10090 GN = Kng2 PE = 1 | | | | | |
| SV = 1 | | | | | |
| Ig-like domain-containing protein | Q8HWB2_MOUSE | 40 | kDa | 6.4766 | 8.7034 |
| OS = Mus musculus (Mouse) OX = 10090 | | | | | |
| GN = H2-Q4 PE = 1 SV = 1 | | | | | |
| Fetuin-B OS = Mus musculus (Mouse) | FETUB_MOUSE | 43 | kDa | 10.178 | 8.7034 |
| OX = 10090 GN = Fetub PE = 1 SV = 1 | | | | | |
| Properdin OS = Mus musculus (Mouse) | PROP_MOUSE | 50 | kDa | 8.327 | 8.7034 |
| OX = 10090 GN = Cfp PE = 1 SV = 2 | | | | | |
| Ig gamma-2B chain C region OS = Mus | IGG2B_MOUSE | 44 | kDa | 10.178 | 8.7034 |
| musculus (Mouse) OX = 10090 GN = Igh-3 | | | | | |
| PE = 1 SV = 3 | | | | | |
| Complement component C8 alpha chain | CO8A_MOUSE | 66 | kDa | 3.7009 | 8.7034 |
| OS = Mus musculus (Mouse) OX = 10090 | | | | | |
| GN = C8a PE = 1 SV = 1 | | | | | |
| Cluster of Heat shock cognate 71 kDa | HSP7C_MOUSE | 71 | kDa | 2.7757 | 8.7034 |
| protein OS = Mus musculus (Mouse) | [2] | | | | |
| OX = 10090 GN = Hspa8 PE = 1 SV = 1 | | | | | |
| (HSP7C_MOUSE) | | | | | |
| Serum amyloid A-1 protein OS = Mus | SAA1_MOUSE | 14 | kDa | 24.056 | 7.6154 |
| musculus (Mouse) OX = 10090 GN = Saa1 | | | | | |
| PE = 1 SV = 2 | | | | | |
| Immunoglobulin kappa constant | IGKC_MOUSE | 12 | kDa | 11.103 | 7.6154 |
| OS = Mus musculus (Mouse) OX = 10090 | | | | | |
| GN = Igkc PE = 1 SV = 2 | | | | | |
| Glutathione peroxidase 3 OS = Mus | GPX3_MOUSE | 25 | kDa | 6.4766 | 7.6154 |
| musculus (Mouse) OX = 10090 GN = Gpx3 | | | | | |
| PE = 1 SV = 2 | | | | | |
| Heat shock cognate 71 kDa protein | HSP7C_MOUSE | 71 | kDa | 2.7757 | 7.6154 |
| OS = Mus musculus (Mouse) OX = 10090 | | | | | |
| GN = Hspa8 PE = 1 SV = 1 | | | | | |
| Alpha-1-antitrypsin 1-5 OS = Mus | A1AT5_MOUSE | 46 | kDa | 21.28 | 6.5275 |
| musculus (Mouse) OX = 10090 | | | | | |
| GN = Serpina1e PE = 1 SV = 1 | | | | | |
| Apolipoprotein A-II OS = Mus musculus | APOA2_MOUSE | 11 | kDa | 14.804 | 6.5275 |
| (Mouse) OX = 10090 GN = Apoa2 PE = 1 | | | | | |
| SV = 2 | | | | | |
| Talin-1 OS = Mus musculus (Mouse) | TLN1_MOUSE | 270 | kDa | 9.2523 | 6.5275 |
| OX = 10090 GN = Tln1 PE = 1 SV = 2 | | | | | |
| Coagulation factor V OS = Mus musculus | FA5_MOUSE | 247 | kDa | 4.6261 | 6.5275 |
| (Mouse) OX = 10090 GN = F5 PE = 1 SV = 1 | | | | | |
| Carboxypeptidase N subunit 2 OS = Mus | CPN2_MOUSE | 60 | kDa | 7.4018 | 6.5275 |
| musculus (Mouse) OX = 10090 GN = Cpn2 | | | | | |
| PE = 1 SV = 2 | | | | | |
| Fibrinogen alpha chain OS = Mus | FIBA_MOUSE | 87 | kDa | 5.5514 | 6.5275 |
| musculus (Mouse) OX = 10090 GN = Fga | | | | | |
| PE = 1 SV = 1 | | | | | |
| Ig-like domain-containing protein | A0A075B5M7_MOUSE | 10 | kDa | 8.327 | 6.5275 |
| OS = Mus musculus (Mouse) OX = 10090 | | | | | |
| GN = Igkv5-39 PE = 1 SV = 7 | | | | | |
| Coagulation factor X OS = Mus musculus | FA10_MOUSE | 54 | kDa | 4.6261 | 6.5275 |
| (Mouse) OX = 10090 GN = F10 PE = 1 SV = 1 | | | | | |
| Immunoglobulin heavy constant gamma | A0A1Y7VJN6_MOUSE | 44 | kDa | 2.7757 | 6.5275 |
| 3 OS = Mus musculus (Mouse) OX = 10090 | (+1) | | | | |
| GN = Ighg3 PE = 4 SV = 1 | | | | | |
| Carbonic anhydrase 1 OS = Mus musculus | CAH1_MOUSE | 28 | kDa | 0 | 6.5275 |
| (Mouse) OX = 10090 GN = Ca1 PE = 1 SV = 4 | | | | | |
| Keratin, type II cytoskeletal 5 OS = Mus | K2C5_MOUSE | 62 | kDa | 10.178 | 5.4396 |
| musculus (Mouse) OX = 10090 GN = Krt5 | | | | | |
| PE = 1 SV = 1 | | | | | |
| Apolipoprotein D OS = Mus musculus | APOD_MOUSE | 22 | kDa | 15.729 | 5.4396 |
| (Mouse) OX = 10090 GN = Apod PE = 1 | | | | | |
| SV = 1 | | | | | |
| Ig gamma-1 chain C region, membrane- | IGH1M_MOUSE | 43 | kDa | 16.654 | 5.4396 |
| bound form OS = Mus musculus (Mouse) | | | | | |
| OX = 10090 GN = Ighg1 PE = 1 SV = 2 | | | | | |
| Apolipoprotein C-IV OS = Mus musculus | APOC4_MOUSE | 14 | kDa | 12.028 | 5.4396 |
| (Mouse) OX = 10090 GN = Apoc4 PE = 1 | | | | | |
| SV = 1 | | | | | |
| Alpha-2-antiplasmin OS = Mus musculus | A2AP_MOUSE | 55 | kDa | 1.8505 | 5.4396 |
| (Mouse) OX = 10090 GN = Serpinf2 PE = 1 | | | | | |
| SV = 1 | | | | | |
| Beta-2-microglobulin OS = Mus musculus | B2MG_MOUSE | 14 | kDa | 5.5514 | 5.4396 |
| (Mouse) OX = 10090 GN = B2m PE = 1 | | | | | |
| SV = 2 | | | | | |
| Immunoglobulin heavy constant gamma | F6TQW2_MOUSE | 44 | kDa | 4.6261 | 5.4396 |
| 2C OS = Mus musculus (Mouse) | | | | | |
| OX = 10090 GN = Ighg2c PE = 1 SV = 2 | | | | | |
| Alpha-1-acid glycoprotein 1 OS = Mus | A1AG1_MOUSE | 24 | kDa | 3.7009 | 5.4396 |
| musculus (Mouse) OX = 10090 GN = Orm1 | | | | | |
| PE = 1 SV = 1 | | | | | |
| Complement component C8 beta chain | CO8B_MOUSE | 66 | kDa | 4.6261 | 5.4396 |
| OS = Mus musculus (Mouse) OX = 10090 | | | | | |
| GN = C8b PE = 1 SV = 1 | | | | | |
| N-acetylmuramoyl-L-alanine amidase | PGRP2_MOUSE | 58 | kDa | 0.92523 | 5.4396 |
| OS = Mus musculus (Mouse) OX = 10090 | | | | | |
| GN = Pglyrp2 PE = 1 SV = 1 | | | | | |
| Vitamin K-dependent protein Z OS = Mus | PROZ_MOUSE | 44 | kDa | 0.92523 | 5.4396 |
| musculus (Mouse) OX = 10090 GN = Proz | | | | | |
| PE = 1 SV = 1 | | | | | |
| Cluster of Filamin-A OS = Mus musculus | FLNA_MOUSE | 281 | kDa | 15.729 | 4.3517 |
| (Mouse) OX = 10090 GN = Flna PE = 1 SV = 5 | [3] | | | | |
| (FLNA_MOUSE) | | | | | |
| Filamin-A OS = Mus musculus (Mouse) | FLNA_MOUSE | 281 | kDa | 15.729 | 4.3517 |
| OX = 10090 GN = Flna PE = 1 SV = 5 | | | | | |
| BPI fold-containing family A member 2 | BPIA2_MOUSE | 25 | kDa | 26.832 | 4.3517 |
| OS = Mus musculus (Mouse) OX = 10090 | | | | | |
| GN = Bpifa2 PE = 1 SV = 1 | | | | | |
| Serum paraoxonase/arylesterase 1 | PON1_MOUSE | 40 | kDa | 15.729 | 4.3517 |
| OS = Mus musculus (Mouse) OX = 10090 | | | | | |
| GN = Pon1 PE = 1 SV = 2 | | | | | |
| Serum amyloid A-4 protein OS = Mus | SAA4_MOUSE | 15 | kDa | 10.178 | 4.3517 |
| musculus (Mouse) OX = 10090 GN = Saa4 | | | | | |
| PE = 1 SV = 2 | | | | | |
| Epidermal growth factor receptor | EGFR_MOUSE | 135 | kDa | 6.4766 | 4.3517 |
| OS = Mus musculus (Mouse) OX = 10090 | | | | | |
| GN = Egfr PE = 1 SV = 1 | | | | | |
| Zinc-alpha-2-glycoprotein OS = Mus | ZA2G_MOUSE | 35 | kDa | 3.7009 | 4.3517 |
| musculus (Mouse) OX = 10090 GN = Azgp1 | | | | | |
| PE = 1 SV = 2 | | | | | |
| Phosphatidylcholine-sterol | LCAT_MOUSE | 50 | kDa | 6.4766 | 4.3517 |
| acyltransferase OS = Mus musculus | | | | | |
| (Mouse) OX = 10090 GN = Lcat PE = 1 SV = 2 | | | | | |
| Mannose-binding protein C OS = Mus | MBL2_MOUSE | 26 | kDa | 5.5514 | 4.3517 |
| musculus (Mouse) OX = 10090 GN = Mbl2 | | | | | |
| PE = 1 SV = 2 | | | | | |
| Carboxypeptidase N catalytic chain | CBPN_MOUSE | 52 | kDa | 1.8505 | 4.3517 |
| OS = Mus musculus (Mouse) OX = 10090 | | | | | |
| GN = Cpn1 PE = 1 SV = 1 | | | | | |
| SCY domain-containing protein OS = Mus | Q9EQI5_MOUSE | 12 | kDa | 1.8505 | 4.3517 |
| musculus (Mouse) OX = 10090 GN = Ppbp | | | | | |
| PE = 1 SV = 1 | | | | | |
| Interleukin-1 receptor accessory protein | IL1AP_MOUSE | 66 | kDa | 0.92523 | 4.3517 |
| OS = Mus musculus (Mouse) OX = 10090 | | | | | |
| GN = Il1rap PE = 1 SV = 1 | | | | | |
| Immunoglobulin J chain OS = Mus | IGJ_MOUSE | 18 | kDa | 2.7757 | 4.3517 |
| musculus (Mouse) OX = 10090 GN = Jchain | | | | | |
| PE = 1 SV = 4 | | | | | |
| Complement C1q subcomponent subunit | C1QA_MOUSE | 26 | kDa | 0 | 4.3517 |
| A OS = Mus musculus (Mouse) OX = 10090 | | | | | |
| GN = C1qa PE = 1 SV = 2 | | | | | |
| Keratin, type II cytoskeletal 2 | K22E_MOUSE | 71 | kDa | 5.5514 | 3.2638 |
| epidermal OS = Mus musculus (Mouse) | | | | | |
| OX = 10090 GN = Krt2 PE = 1 SV = 1 | | | | | |
| Keratin, type II cytoskeletal 1 OS = Mus | K2C1_MOUSE | 66 | kDa | 12.953 | 3.2638 |
| musculus (Mouse) OX = 10090 GN = Krt1 | | | | | |
| PE = 1 SV = 4 | | | | | |
| IF rod domain-containing protein | E9Q1Z0_MOUSE | 58 | kDa | 4.6261 | 3.2638 |
| OS = Mus musculus (Mouse) OX = 10090 | | | | | |
| GN = Krt90 PE = 1 SV = 1 | | | | | |
| Keratin, type II cytoskeletal 1b | K2C1B_MOUSE | 61 | kDa | 7.4018 | 3.2638 |
| OS = Mus musculus (Mouse) OX = 10090 | | | | | |
| GN = Krt77 PE = 1 SV = 1 | | | | | |
| Keratin, type II cytoskeletal 75 | K2C75_MOUSE | 60 | kDa | 5.5514 | 3.2638 |
| OS = Mus musculus (Mouse) OX = 10090 | | | | | |
| GN = Krt75 PE = 1 SV = 1 | | | | | |
| Carboxylic ester hydrolase OS = Mus | E9PYP1_MOUSE | 62 | kDa | 0 | 3.2638 |
| musculus (Mouse) OX = 10090 GN = Ces1a | | | | | |
| PE = 1 SV = 1 | | | | | |
| Fibrinogen gamma chain OS = Mus | FIBG_MOUSE | 49 | kDa | 4.6261 | 3.2638 |
| musculus (Mouse) OX = 10090 GN = Fgg | | | | | |
| PE = 1 SV = 1 | | | | | |
| Cluster of Keratin, type I cytoskeletal 10 | K1C10_MOUSE | 58 | kDa | 7.4018 | 3.2638 |
| OS = Mus musculus (Mouse) OX = 10090 | [2] | | | | |
| GN = Krt10 PE = 1 SV = 3 | | | | | |
| (K1C10_MOUSE) | | | | | |
| Keratin, type I cytoskeletal 10 | K1C10_MOUSE | 58 | kDa | 7.4018 | 3.2638 |
| OS = Mus musculus (Mouse) OX = 10090 | | | | | |
| GN = Krt10 PE = 1 SV = 3 | | | | | |
| Serum amyloid A-2 protein OS = Mus | SAA2_MOUSE | 14 | kDa | 12.953 | 3.2638 |
| musculus (Mouse) OX = 10090 GN = Saa2 | | | | | |
| PE = 1 SV = 1 | | | | | |
| Transthyretin OS = Mus musculus | TTHY_MOUSE | 16 | kDa | 7.4018 | 3.2638 |
| (Mouse) OX = 10090 GN = Ttr PE = 1 SV = 1 | | | | | |
| Carbonic anhydrase 2 OS = Mus musculus | CAH2_MOUSE | 29 | kDa | 2.7757 | 3.2638 |
| (Mouse) OX = 10090 GN = Ca2 PE = 1 SV = 4 | | | | | |
| Immunoglobulin heavy constant alpha | A0A0A6YXW6_MOUSE | 42 | kDa | 2.7757 | 3.2638 |
| OS = Mus musculus (Mouse) OX = 10090 | | | | | |
| GN = Igha PE = 1 SV = 1 | | | | | |
| Plasma protease C1 inhibitor OS = Mus | IC1_MOUSE | 56 | kDa | 5.5514 | 3.2638 |
| musculus (Mouse) OX = 10090 | | | | | |
| GN-Serping1 PE = 1 SV = 3 | | | | | |
| Transitional endoplasmic reticulum | TERA_MOUSE | 89 | kDa | 0.92523 | 3.2638 |
| ATPase OS = Mus musculus (Mouse) | | | | | |
| OX = 10090 GN = Vcp PE = 1 SV = 4 | | | | | |
| Coagulation factor XII OS = Mus | FA12_MOUSE | 66 | kDa | 2.7757 | 3.2638 |
| musculus (Mouse) OX = 10090 GN = F12 | | | | | |
| PE = 1 SV = 2 | | | | | |
| Glyceraldehyde-3-phosphate | G3P_MOUSE | 36 | kDa | 2.7757 | 3.2638 |
| dehydrogenase OS = Mus musculus | | | | | |
| (Mouse) OX = 10090 GN = Gapdh PE = 1 | | | | | |
| SV = 2 | | | | | |
| Mannan-binding lectin serine protease 2 | MASP2_MOUSE | 76 | kDa | 0.92523 | 3.2638 |
| OS = Mus musculus (Mouse) OX = 10090 | | | | | |
| GN = Masp2 PE = 1 SV = 1 | | | | | |
| Alpha-1-acid glycoprotein 2 OS = Mus | A1AG2_MOUSE | 24 | kDa | 2.7757 | 3.2638 |
| musculus (Mouse) OX = 10090 GN = Orm2 | | | | | |
| PE = 1 SV = 1 | | | | | |
| sp\|Q8BXA7\|PHLP2_MOUSE-DECOY | sp\|Q8BXA7\|PHLP2_MOUSE- | ? | | 0 | 3.2638 |
| | DECOY | | | | |
| Keratin, type II cytoskeletal 73 | K2C73_MOUSE | 59 | kDa | 7.4018 | 2.1758 |
| OS = Mus musculus (Mouse) OX = 10090 | | | | | |
| GN = Krt73 PE = 1 SV = 1 | | | | | |
| IF rod domain-containing protein | A0A2R8VHP3_MOUSE | 58 | kDa | 5.5514 | 2.1758 |
| OS = Mus musculus (Mouse) OX = 10090 | | | | | |
| GN = Gm5478 PE = 1 SV = 1 | | | | | |
| Keratin, type II cytoskeletal 2 oral | K22O_MOUSE | 63 | kDa | 1.8505 | 2.1758 |
| OS = Mus musculus (Mouse) OX = 10090 | | | | | |
| GN = Krt76 PE = 1 SV = 1 | | | | | |
| Apolipoprotein C-III OS = Mus musculus | APOC3_MOUSE | 11 | kDa | 30.533 | 2.1758 |
| (Mouse) OX = 10090 GN = Apoc3 PE = 1 | | | | | |
| SV = 2 | | | | | |
| Predicted gene 4788 OS = Mus musculus | E9Q8B5_MOUSE | 99 | kDa | 21.28 | 2.1758 |
| (Mouse) OX = 10090 GN = Gm4788 PE = 1 | | | | | |
| SV = 1 | | | | | |
| Endoplasmic reticulum chaperone BiP | BIP_MOUSE | 72 | kDa | 0.92523 | 2.1758 |
| OS = Mus musculus (Mouse) OX = 10090 | | | | | |
| GN = Hspa5 PE = 1 SV = 3 | | | | | |
| IF rod domain-containing protein | E9Q0F0_MOUSE | 112 | kDa | 8.327 | 2.1758 |
| OS = Mus musculus (Mouse) OX = 10090 | | | | | |
| GN = Krt78 PE = 1 SV = 1 | | | | | |
| Selenoprotein P OS = Mus musculus | SEPP1_MOUSE | 43 | kDa | 2.7757 | 2.1758 |
| (Mouse) OX = 10090 GN = Selenop PE = 1 | | | | | |
| SV = 3 | | | | | |
| Platelet-activating factor acetylhydrolase | PAFA_MOUSE | 49 | kDa | 7.4018 | 2.1758 |
| OS = Mus musculus (Mouse) OX = 10090 | | | | | |
| GN = Pla2g7 PE = 1 SV = 2 | | | | | |
| Complement component C9 OS = Mus | CO9_MOUSE | 62 | kDa | 3.7009 | 2.1758 |
| musculus (Mouse) OX = 10090 GN = C9 | | | | | |
| PE = 1 SV = 2 | | | | | |
| sp\|P46656\|ADX_MOUSE-DECOY | sp\|P46656\|ADX_MOUSE- | ? | | 0 | 2.1758 |
| | DECOY | | | | |
| Corticosteroid-binding globulin OS = Mus | CBG_MOUSE | 45 | kDa | 1.8505 | 2.1758 |
| musculus (Mouse) OX = 10090 | | | | | |
| GN = Serpina6 PE = 1 SV = 1 | | | | | |
| Platelet factor 4 OS = Mus musculus | PLF4_MOUSE | 11 | kDa | 2.7757 | 2.1758 |
| (Mouse) OX = 10090 GN = Pf4 PE = 1 SV = 1 | | | | | |
| Cluster of Alpha-amylase 1 OS = Mus | AMY1_MOUSE | 58 | kDa | 0.92523 | 2.1758 |
| musculus (Mouse) OX = 10090 GN = Amy1 | [3] | | | | |
| PE = 1 SV = 2 (AMY1_MOUSE) | | | | | |
| Alpha-amylase 1 OS = Mus musculus | AMY1_MOUSE | 58 | kDa | 0.92523 | 2.1758 |
| (Mouse) OX = 10090 GN = Amy1 PE = 1 | | | | | |
| SV = 2 | | | | | |
| Heparin cofactor 2 OS = Mus musculus | HEP2_MOUSE | 54 | kDa | 1.8505 | 2.1758 |
| (Mouse) OX = 10090 GN = Serpind1 PE = 1 | | | | | |
| SV = 1 | | | | | |
| Ig-like domain-containing protein | A0A075B5M2_MOUSE | 10 | kDa | 3.7009 | 2.1758 |
| OS = Mus musculus (Mouse) OX = 10090 | | | | | |
| GN = Igkv4-61 PE = 4 SV = 7 | | | | | |
| Cluster of Tubulin alpha-4A chain | TBA4A_MOUSE | 50 | kDa | 6.4766 | 2.1758 |
| OS = Mus musculus (Mouse) OX = 10090 | [2] | | | | |
| GN = Tuba4a PE = 1 SV = 1 | | | | | |
| (TBA4A_MOUSE) | | | | | |
| Tubulin alpha-4A chain OS = Mus | TBA4A_MOUSE | 50 | kDa | 3.7009 | 2.1758 |
| musculus (Mouse) OX = 10090 | | | | | |
| GN = Tuba4a PE = 1 SV = 1 | | | | | |
| Complement factor D OS = Mus musculus | CFAD_MOUSE | 28 | kDa | 2.7757 | 2.1758 |
| (Mouse) OX = 10090 GN = Cfd PE = 1 SV = 1 | | | | | |
| Retinol-binding protein 4 OS = Mus | RET4_MOUSE | 23 | kDa | 2.7757 | 2.1758 |
| musculus (Mouse) OX = 10090 GN = Rbp4 | | | | | |
| PE = 1 SV = 2 | | | | | |
| 14-3-3 protein zeta/delta OS = Mus | 1433Z_MOUSE | 28 | kDa | 2.7757 | 2.1758 |
| musculus (Mouse) OX = 10090 | | | | | |
| GN = Ywhaz PE = 1 SV = 1 | | | | | |
| Secreted phosphoprotein 24 OS = Mus | SPP24_MOUSE | 23 | kDa | 1.8505 | 2.1758 |
| musculus (Mouse) OX = 10090 GN = Spp2 | | | | | |
| PE = 1 SV = 2 | | | | | |
| Macrophage colony-stimulating factor 1 | CSF1R_MOUSE | 109 | kDa | 1.8505 | 2.1758 |
| receptor OS = Mus musculus (Mouse) | | | | | |
| OX = 10090 GN = Csf1r PE = 1 SV = 3 | | | | | |
| Lumican OS = Mus musculus (Mouse) | LUM_MOUSE | 38 | kDa | 0.92523 | 2.1758 |
| OX = 10090 GN = Lum PE = 1 SV = 2 | | | | | |
| 55 kDa erythrocyte membrane protein | EM55_MOUSE | 52 | kDa | 0 | 2.1758 |
| OS = Mus musculus (Mouse) OX = 10090 | | | | | |
| GN = Mpp1 PE = 1 SV = 1 | | | | | |
| sp\|Q9WTU0\|PHF2_MOUSE-DECOY | sp\|Q9WTU0\|PHF2_MOUSE- | ? | | 0.92523 | 2.1758 |
| | DECOY | | | | |
| sp\|P59242\|CING_MOUSE-DECOY | sp\|P59242\|CING_MOUSE- | ? | | 0 | 2.1758 |
| | DECOY | | | | |
| C-reactive protein OS = Mus musculus | CRP_MOUSE | 25 | kDa | 1.8505 | 2.1758 |
| (Mouse) OX = 10090 GN = Crp PE = 1 SV = 2 | | | | | |
| Coagulation factor IX OS = Mus musculus | FA9_MOUSE | 53 | kDa | 0 | 2.1758 |
| (Mouse) OX = 10090 GN = F9 PE = 2 SV = 3 | | | | | |
| Peroxiredoxin-2 OS = Mus musculus | PRDX2_MOUSE | 22 | kDa | 0.92523 | 2.1758 |
| (Mouse) OX = 10090 GN = Prdx2 PE = 1 | | | | | |
| SV = 3 | | | | | |
| Vitamin K-dependent protein S OS = Mus | PROS_MOUSE | 75 | kDa | 0 | 2.1758 |
| musculus (Mouse) OX = 10090 GN = Pros1 | | | | | |
| PE = 2 SV = 1 | | | | | |
| sp\|Q571H0\|NPA1P_MOUSE-DECOY | sp\|Q571H0\|NPA1P_MOUSE- | ? | | 0 | 2.1758 |
| | DECOY | | | | |
| Ficolin-1 OS = Mus musculus (Mouse) | FCN1_MOUSE | 36 | kDa | 0 | 2.1758 |
| OX = 10090 GN = Fcn1 PE = 1 SV = 1 | | | | | |
| Ig lambda-2 chain C region OS = Mus | LAC2_MOUSE | 11 | kDa | 0 | 2.1758 |
| musculus (Mouse) OX = 10090 GN = Iglc2 | | | | | |
| PE = 1 SV = 1 | | | | | |
| Profilin-1 OS = Mus musculus (Mouse) | PROF1_MOUSE | 15 | kDa | 0 | 2.1758 |
| OX = 10090 GN = Pfn1 PE = 1 SV = 2 | | | | | |
| Extracellular superoxide dismutase | SODE_MOUSE | 27 | kDa | 0 | 2.1758 |
| [Cu—Zn] OS = Mus musculus (Mouse) | | | | | |
| OX = 10090 GN = Sod3 PE = 1 SV = 1 | | | | | |
| Complement C1q subcomponent subunit | C1QC_MOUSE | 26 | kDa | 0 | 2.1758 |
| C OS = Mus musculus (Mouse) OX = 10090 | | | | | |
| GN = C1qc PE = 1 SV = 2 | | | | | |
| Biotinidase OS = Mus musculus (Mouse) | BTD_MOUSE | 58 | kDa | 0 | 2.1758 |
| OX = 10090 GN = Btd PE = 1 SV = 2 | | | | | |
| Keratin, type II cytoskeletal 79 | K2C79_MOUSE | 58 | kDa | 2.7757 | 1.0879 |
| OS = Mus musculus (Mouse) OX = 10090 | | | | | |
| GN = Krt79 PE = 1 SV = 2 | | | | | |
| Keratin, type II cytoskeletal 8 OS = Mus | K2C8_MOUSE | 55 | kDa | 1.8505 | 1.0879 |
| musculus (Mouse) OX = 10090 GN = Krt8 | | | | | |
| PE = 1 SV = 4 | | | | | |
| Keratin, type II cytoskeletal 74 | K2C74_MOUSE | 55 | kDa | 0 | 1.0879 |
| OS = Mus musculus (Mouse) OX = 10090 | | | | | |
| GN = Krt74 PE = 3 SV = 1 | | | | | |
| Ankyrin-3 OS = Mus musculus (Mouse) | ANK3_MOUSE | 214 | kDa | 0 | 1.0879 |
| OX = 10090 GN = Ank3 PE = 1 SV = 1 | | | | | |
| Ankyrin-2 OS = Mus musculus (Mouse) | ANK2_MOUSE | 426 | kDa | 0 | 1.0879 |
| OX = 10090 GN = Ank2 PE = 1 SV = 2 | | | | | |
| C4b-binding protein OS = Mus musculus | C4BPA_MOUSE | 52 | kDa | 20.355 | 1.0879 |
| (Mouse) OX = 10090 GN = C4bpa PE = 1 | | | | | |
| SV = 3 | | | | | |
| Apolipoprotein C-I OS = Mus musculus | APOC1_MOUSE | 10 | kDa | 10.178 | 1.0879 |
| (Mouse) OX = 10090 GN = Apoc1 PE = 1 | | | | | |
| SV = 1 | | | | | |
| Keratin, type I cytoskeletal 16 | K1C16_MOUSE | 52 | kDa | 1.8505 | 1.0879 |
| OS = Mus musculus (Mouse) OX = 10090 | | | | | |
| GN = Krt16 PE = 1 SV = 3 | | | | | |
| Pigment epithelium-derived factor | PEDF_MOUSE | 46 | kDa | 6.4766 | 1.0879 |
| OS = Mus musculus (Mouse) OX = 10090 | | | | | |
| GN = Serpinf1 PE = 1 SV = 2 | | | | | |
| Endogenous retroviral sequence 3 | Q9DAX3_MOUSE | 23 | kDa | 0 | 1.0879 |
| OS = Mus musculus (Mouse) OX = 10090 | | | | | |
| GN = Erv3 PE = 1 SV = 1 | | | | | |
| Predicted gene 382 OS = Mus musculus | B1AXN3_MOUSE | 142 | kDa | 0 | 1.0879 |
| (Mouse) OX = 10090 GN = Gm382 PE = 4 | | | | | |
| SV = 1 | | | | | |
| Cluster of Myosin-9 OS = Mus musculus | MYH9_MOUSE | 226 | kDa | 4.6261 | 1.0879 |
| (Mouse) OX = 10090 GN = Myh9 PE = 1 | [2] | | | | |
| SV = 4 (MYH9_MOUSE) | | | | | |
| Myosin-9 OS = Mus musculus (Mouse) | MYH9_MOUSE | 226 | kDa | 4.6261 | 1.0879 |
| OX = 10090 GN = Myh9 PE = 1 SV = 4 | | | | | |
| Myosin-11 OS = Mus musculus (Mouse) | MYH11_MOUSE | 227 | kDa | 1.8505 | 1.0879 |
| OX = 10090 GN = Myh11 PE = 1 SV = 1 | | | | | |
| sp\|P98083\|SHC1_MOUSE-DECOY | sp\|P98083\|SHC1_MOUSE- | ? | | 0.92523 | 1.0879 |
| | DECOY | | | | |
| Hepatocyte growth factor activator | HGFA_MOUSE | 71 | kDa | 2.7757 | 1.0879 |
| OS = Mus musculus (Mouse) OX = 10090 | | | | | |
| GN = Hgfac PE = 1 SV = 1 | | | | | |
| Pancreatic alpha-amylase OS = Mus | AMYP_MOUSE | 57 | kDa | 0 | 1.0879 |
| musculus (Mouse) OX = 10090 GN = Amy2 | | | | | |
| PE = 1 SV = 2 | | | | | |
| Aamy domain-containing protein | E9PV85_MOUSE | 28 | kDa | 0 | 1.0879 |
| OS = Mus musculus (Mouse) OX = 10090 | | | | | |
| GN = Amy2a1 PE = 1 SV = 1 | | | | | |
| Ig-like domain-containing protein | A0A075B5T2_MOUSE | 13 | kDa | 0.92523 | 1.0879 |
| OS = Mus musculus (Mouse) OX = 10090 | | | | | |
| GN = Ighv6-3 PE = 4 SV = 7 | | | | | |
| Keratin, type I cytoskeletal 28 OS = Mus | K1C28_MOUSE | 50 | kDa | 5.5514 | 1.0879 |
| musculus (Mouse) OX = 10090 GN = Krt28 | | | | | |
| PE = 1 SV = 1 | | | | | |
| Spectrin alpha chain, non-erythrocytic 1 | SPTN1_MOUSE | 285 | kDa | 0.92523 | 1.0879 |
| OS = Mus musculus (Mouse) OX = 10090 | | | | | |
| GN = Sptan1 PE = 1 SV = 4 | | | | | |
| Extracellular matrix protein 1 OS = Mus | ECM1_MOUSE | 63 | kDa | 3.7009 | 1.0879 |
| musculus (Mouse) OX = 10090 GN = Ecm1 | | | | | |
| PE = 1 SV = 2 | | | | | |
| Fibrinogen beta chain OS = Mus musculus | FIBB_MOUSE | 55 | kDa | 2.7757 | 1.0879 |
| (Mouse) OX = 10090 GN = Fgb PE = 1 SV = 1 | | | | | |
| Mannose-binding protein A OS = Mus | MBL1_MOUSE | 25 | kDa | 3.7009 | 1.0879 |
| musculus (Mouse) OX = 10090 GN = Mbl1 | | | | | |
| PE = 1 SV = 1 | | | | | |
| Carboxypeptidase B2 OS = Mus musculus | CBPB2_MOUSE | 49 | kDa | 0.92523 | 1.0879 |
| (Mouse) OX = 10090 GN = Cpb2 PE = 1 | | | | | |
| SV = 1 | | | | | |
| Tubulin alpha-1B chain OS = Mus | TBA1B_MOUSE | 50 | kDa | 4.6261 | 1.0879 |
| musculus (Mouse) OX = 10090 | | | | | |
| GN = Tuba1b PE = 1 SV = 2 | | | | | |
| Complement component C8 gamma | CO8G_MOUSE | 23 | kDa | 2.7757 | 1.0879 |
| chain OS = Mus musculus (Mouse) | | | | | |
| OX = 10090 GN = C8g PE = 1 SV = 1 | | | | | |
| Keratin, type I cytoskeletal 42 OS = Mus | K1C42_MOUSE | 50 | kDa | 0.92523 | 1.0879 |
| musculus (Mouse) OX = 10090 GN = Krt42 | | | | | |
| PE = 1 SV = 1 | | | | | |
| Protein PRRC2B OS = Mus musculus | PRC2B_MOUSE | 161 | kDa | 0.92523 | 1.0879 |
| (Mouse) OX = 10090 GN = Prrc2b PE = 1 | | | | | |
| SV = 1 | | | | | |
| Lysosomal alpha-mannosidase OS = Mus | MA2B1_MOUSE | 115 | kDa | 0 | 1.0879 |
| musculus (Mouse) OX = 10090 | | | | | |
| GN = Man2b1 PE = 1 SV = 4 | | | | | |
| sp\|Q6RUT8\|CC154_MOUSE-DECOY | sp\|Q6RUT8\|CC154_MOUSE- | ? | | 0 | 1.0879 |
| | DECOY | | | | |
| sp\|Q8BMD6\|TM260_MOUSE-DECOY | sp\|Q8BMD6\|TM260_MOUSE- | ? | | 0.92523 | 1.0879 |
| | DECOY | | | | |
| Ras-related protein Rap-1b OS = Mus | RAP1B_MOUSE | 21 | kDa | 1.8505 | 1.0879 |
| musculus (Mouse) OX = 10090 GN = Rap1b | | | | | |
| PE = 1 SV = 2 | | | | | |
| Alpha-actinin-1 OS = Mus musculus | ACTN1_MOUSE | 103 | kDa | 0.92523 | 1.0879 |
| (Mouse) OX = 10090 GN = Actn1 PE = 1 | | | | | |
| SV = 1 | | | | | |
| Proteoglycan 4 OS = Mus musculus | PRG4_MOUSE | 116 | kDa | 0.92523 | 1.0879 |
| (Mouse) OX = 10090 GN = Prg4 PE = 1 | | | | | |
| SV = 2 | | | | | |
| Ig-like domain-containing protein | A0A0B4J1J7_MOUSE | 11 | kDa | 1.8505 | 1.0879 |
| OS = Mus musculus (Mouse) OX = 10090 | | | | | |
| GN = Ighv1-82 PE = 1 SV = 1 | | | | | |
| von Willebrand factor OS = Mus musculus | VWF_MOUSE | 309 | kDa | 0.92523 | 1.0879 |
| (Mouse) OX = 10090 GN = Vwf PE = 1 SV = 2 | | | | | |
| Serglycin OS = Mus musculus (Mouse) | SRGN_MOUSE | 17 | kDa | 1.8505 | 1.0879 |
| OX = 10090 GN = Srgn PE = 1 SV = 1 | | | | | |
| sp\|A2AAY5\|SPD2B_MOUSE-DECOY | sp\|A2AAY5\|SPD2B_MOUSE- | ? | | 0 | 1.0879 |
| | DECOY | | | | |
| sp\|Q9JLV2\|TP4AP_MOUSE-DECOY | sp\|Q9JLV2\|TP4AP_MOUSE- | ? | | 0 | 1.0879 |
| | DECOY | | | | |
| Hyaluronan-binding protein 2 OS = Mus | HABP2_MOUSE | 62 | kDa | 0 | 1.0879 |
| musculus (Mouse) OX = 10090 GN = Habp2 | | | | | |
| PE = 1 SV = 2 | | | | | |
| Disks large homolog 5 OS = Mus musculus | DLG5_MOUSE | 214 | kDa | 0 | 1.0879 |
| (Mouse) OX = 10090 GN = Dlg5 PE = 1 | | | | | |
| SV = 1 | | | | | |
| sp\|Q7TSJ2\|MAP6_MOUSE-DECOY | sp\|Q7TSJ2\|MAP6_MOUSE- | ? | | 0 | 1.0879 |
| | DECOY | | | | |
| Amyloid-beta A4 precursor protein- | APBB2_MOUSE | 83 | kDa | 0 | 1.0879 |
| binding family B member 2 OS = Mus | | | | | |
| musculus (Mouse) OX = 10090 GN = Apbb2 | | | | | |
| PE = 1 SV = 2 | | | | | |
| sp\|Q9Z1P8\|ANGL4_MOUSE-DECOY | sp\|Q9Z1P8\|ANGL4_MOUSE- | ? | | 0.92523 | 1.0879 |
| | DECOY | | | | |
| tr\|J3QNP2\|J3QNP2_MOUSE-DECOY | tr\|J3QNP2\|J3QNP2_MOUSE- | ? | | 0 | 1.0879 |
| | DECOY | | | | |
| Insulin-like growth factor-binding | ALS_MOUSE | 67 | kDa | 1.8505 | 1.0879 |
| protein complex acid labile subunit | | | | | |
| OS = Mus musculus (Mouse) OX = 10090 | | | | | |
| GN = Igfals PE = 1 SV = 1 | | | | | |
| Fermitin family homolog 3 OS = Mus | URP2_MOUSE | 76 | kDa | 1.8505 | 1.0879 |
| musculus (Mouse) OX = 10090 | | | | | |
| GN = Fermt3 PE = 1 SV = 1 | | | | | |
| Ig-like domain-containing protein | A0A0G2JE99_MOUSE | 12 | kDa | 1.8505 | 1.0879 |
| OS = Mus musculus (Mouse) OX = 10090 | (+1) | | | | |
| GN = Iglc1 PE = 4 SV = 1 | | | | | |
| Ig kappa chain V-II region 26-10 | KV2A7_MOUSE | 12 | kDa | 1.8505 | 1.0879 |
| OS = Mus musculus (Mouse) OX = 10090 | | | | | |
| GN = — PE = 1 SV = 1 | | | | | |
| Ig kappa chain V-V region MOPC 41 | KV5A7_MOUSE | 14 | kDa | 0.92523 | 1.0879 |
| OS = Mus musculus (Mouse) OX = 10090 | | | | | |
| GN = Gm5571 PE = 1 SV = 1 | | | | | |
| L-selectin OS = Mus musculus (Mouse) | LYAM1_MOUSE | 42 | kDa | 0 | 1.0879 |
| OX = 10090 GN = Sell PE = 1 SV = 1 | | | | | |
| Ig kappa chain V-VI region XRPC 44 | KV6A1_MOUSE | 12 | kDa | 0.92523 | 1.0879 |
| OS = Mus musculus (Mouse) OX = 10090 | (+4) | | | | |
| GN = — PE = 1 SV = 1 | | | | | |
| Leukemia inhibitory factor receptor | LIFR_MOUSE | 123 | kDa | 0.92523 | 1.0879 |
| OS = Mus musculus (Mouse) OX = 10090 | | | | | |
| GN = Lifr PE = 1 SV = 1 | | | | | |
| Platelet glycoprotein Ib alpha chain | GP1BA_MOUSE | 80 | kDa | 0 | 1.0879 |
| OS = Mus musculus (Mouse) OX = 10090 | | | | | |
| GN = Gp1ba PE = 1 SV = 2 | | | | | |
| Hepatocyte growth factor-like protein | HGFL_MOUSE | 81 | kDa | 0 | 1.0879 |
| OS = Mus musculus (Mouse) OX = 10090 | | | | | |
| GN = Mst1 PE = 2 SV = 2 | | | | | |
| Nuclear receptor subfamily 1 group D | NR1D2_MOUSE | 64 | kDa | 0 | 1.0879 |
| member 2 OS = Mus musculus (Mouse) | | | | | |
| OX = 10090 GN = Nr1d2 PE = 1 SV = 1 | | | | | |
| sp\|B2RQE8\|RHG42_MOUSE-DECOY | sp\|B2RQE8\|RHG42_MOUSE- | ? | | 0 | 1.0879 |
| | DECOY | | | | |
| Lysozyme C-2 OS = Mus musculus | LYZ2_MOUSE | 17 | kDa | 0 | 1.0879 |
| (Mouse) OX = 10090 GN = Lyz2 PE = 1 | | | | | |
| SV = 2 | | | | | |
| Tubulin alpha-8 chain OS = Mus | TBA8_MOUSE | 50 | kDa | 1.8505 | 1.0879 |
| musculus (Mouse) OX = 10090 GN = Tuba8 | | | | | |
| PE = 1 SV = 1 | | | | | |
| Vitamin K-dependent protein C OS = Mus | PROC_MOUSE | 52 | kDa | 0 | 1.0879 |
| musculus (Mouse) OX = 10090 GN = Proc | | | | | |
| PE = 1 SV = 2 | | | | | |
| RIKEN cDNA 9530053A07 gene | E9PVG8_MOUSE | 280 | kDa | 0 | 1.0879 |
| OS = Mus musculus (Mouse) OX = 10090 | | | | | |
| GN = 9530053A07Rik PE = 1 SV = 1 | | | | | |
| Ig-like domain-containing protein | A0A075B5S2_MOUSE | 14 | kDa | 0.92523 | 1.0879 |
| OS = Mus musculus (Mouse) OX = 10090 | | | | | |
| GN = Ighv7-1 PE = 1 SV = 2 | | | | | |
| Ig-like domain-containing protein | A0A075B5V8_MOUSE | 11 | kDa | 0.92523 | 1.0879 |
| OS = Mus musculus (Mouse) OX = 10090 | (+1) | | | | |
| GN = Ighv1-47 PE = 4 SV = 1 | | | | | |
| Superoxide dismutase [Cu—Zn] OS = Mus | SODC_MOUSE | 16 | kDa | 0.92523 | 1.0879 |
| musculus (Mouse) OX = 10090 GN = Sod1 | | | | | |
| PE = 1 SV = 2 | | | | | |
| Triosephosphate isomerase OS = Mus | TPIS_MOUSE | 32 | kDa | 0.92523 | 1.0879 |
| musculus (Mouse) OX = 10090 GN = Tpi1 | | | | | |
| PE = 1 SV = 4 | | | | | |
| Anaphylatoxin-like domain-containing | A2AS37_MOUSE | 40 | kDa | 0.92523 | 1.0879 |
| protein OS = Mus musculus (Mouse) | | | | | |
| OX = 10090 GN = AI182371 PE = 1 SV = 2 | | | | | |
| Hepcidin OS = Mus musculus (Mouse) | HEPC_MOUSE | 9 | kDa | 0.92523 | 1.0879 |
| OX = 10090 GN = Hamp PE = 2 SV = 1 | | | | | |
| Peptidyl-prolyl cis-trans isomerase A | PPIA_MOUSE | 18 | kDa | 0 | 1.0879 |
| OS = Mus musculus (Mouse) OX = 10090 | | | | | |
| GN = Ppia PE = 1 SV = 2 | | | | | |
| Metalloproteinase inhibitor 3 OS = Mus | TIMP3_MOUSE | 24 | kDa | 0 | 1.0879 |
| musculus (Mouse) OX = 10090 GN = Timp3 | | | | | |
| PE = 1 SV = 1 | | | | | |
| Ig-like domain-containing protein | A0A140T8M5_MOUSE | 13 | kDa | 0.92523 | 1.0879 |
| OS = Mus musculus (Mouse) OX = 10090 | | | | | |
| GN = Igkv6-15 PE = 1 SV = 2 | | | | | |
| Immunoglobulin heavy variable 1-15 | A0A0A6YXA5_MOUSE | 13 | kDa | 0 | 1.0879 |
| OS = Mus musculus (Mouse) OX = 10090 | | | | | |
| GN = Ighv1-15 PE = 4 SV = 1 | | | | | |
| Progranulin OS = Mus musculus (Mouse) | GRN_MOUSE | 63 | kDa | 0.92523 | 1.0879 |
| OX = 10090 GN = Grn PE = 1 SV = 2 | | | | | |
| Testisin OS = Mus musculus (Mouse) | TEST_MOUSE | 36 | kDa | 0 | 1.0879 |
| OX = 10090 GN = Prss21 PE = 1 SV = 2 | | | | | |
| tr\|Q91X36\|Q91X36_MOUSE-DECOY | tr\|Q91X36\|Q91X36_MOUSE- | ? | | 0 | 1.0879 |
| | DECOY | | | | |
| sp\|Q8VEE4\|RFA1_MOUSE-DECOY | sp\|Q8VEE4\|RFA1_MOUSE- | ? | | 0.92523 | 1.0879 |
| | DECOY | | | | |
| Complement C1r-A subcomponent | C1RA_MOUSE | 80 | kDa | 0 | 1.0879 |
| OS = Mus musculus (Mouse) OX = 10090 | (+1) | | | | |
| GN = C1ra PE = 1 SV = 1 | | | | | |
| Carboxypeptidase Q OS = Mus musculus | CBPQ_MOUSE | 52 | kDa | 0 | 1.0879 |
| (Mouse) OX = 10090 GN = Cpq PE = 1 SV = 1 | | | | | |
| Dematin OS = Mus musculus (Mouse) | DEMA_MOUSE | 45 | kDa | 0 | 1.0879 |
| OX = 10090 GN = Dmtn PE = 1 SV = 1 | | | | | |
| Ig heavy chain V region MOPC 47A | HVM17_MOUSE | 13 | kDa | 0 | 1.0879 |
| OS = Mus musculus (Mouse) OX = 10090 | | | | | |
| GN = — PE = 1 SV = 1 | | | | | |
| T-lymphoma invasion and metastasis- | TIAM1_MOUSE | 178 | kDa | 0 | 1.0879 |
| inducing protein 1 OS = Mus musculus | | | | | |
| (Mouse) OX = 10090 GN = Tiam1 PE = 1 | | | | | |
| SV = 1 | | | | | |
| Ras-related protein Rab-19 OS = Mus | RAB19_MOUSE | 24 | kDa | 0 | 1.0879 |
| musculus (Mouse) OX = 10090 GN = Rab19 | | | | | |
| PE = 1 SV = 3 | | | | | |
| Protein disulfide-isomerase A3 OS = Mus | PDIA3_MOUSE | 57 | kDa | 0 | 1.0879 |
| musculus (Mouse) OX = 10090 GN = Pdia3 | | | | | |
| PE = 1 SV = 2 | | | | | |
| Proto-oncogene tyrosine-protein kinase | RET_MOUSE | 124 | kDa | 0 | 1.0879 |
| receptor Ret OS = Mus musculus (Mouse) | | | | | |
| OX = 10090 GN = Ret PE = 1 SV = 2 | | | | | |
| Ig-like domain-containing protein | A0A075B5J9_MOUSE | 12 | kDa | 0 | 1.0879 |
| OS = Mus musculus (Mouse) OX = 10090 | (+1) | | | | |
| GN = Igkv17-127 PE = 4 SV = 7 | | | | | |
| Monocarboxylate transporter 1 OS = Mus | MOT1_MOUSE | 53 | kDa | 0 | 1.0879 |
| musculus (Mouse) OX = 10090 | | | | | |
| GN = Slc16a1 PE = 1 SV = 1 | | | | | |
| sp\|P67984\|RL22_MOUSE-DECOY | sp\|P67984\|RL22_MOUSE- | ? | | 0 | 1.0879 |
| | DECOY | | | | |
| Phosphatidylinositide phosphatase SAC2 | SAC2_MOUSE | 128 | kDa | 0 | 1.0879 |
| OS = Mus musculus (Mouse) OX = 10090 | | | | | |
| GN = Inpp5f PE = 1 SV = 1 | | | | | |
| Complement factor H-related 2 | A0A668KLU9_MOUSE | 38 | kDa | 16.654 | 0 |
| OS = Mus musculus (Mouse) OX = 10090 | | | | | |
| GN = Cfhr2 PE = 4 SV = 1 | | | | | |
| Serine protease inhibitor A3G | SPA3G_MOUSE | 49 | kDa | 18.505 | 0 |
| OS = Mus musculus (Mouse) OX = 10090 | | | | | |
| GN = Serpina3g PE = 1 SV = 2 | | | | | |
| Neurofilament heavy polypeptide | NFH_MOUSE | 117 | kDa | 0.92523 | 0 |
| OS = Mus musculus (Mouse) OX = 10090 | | | | | |
| GN = Nefh PE = 1 SV = 3 | | | | | |
| H-2 class I histocompatibility antigen, | HA1Z_MOUSE | 20 | kDa | 0.92523 | 0 |
| alpha chain OS = Mus musculus (Mouse) | | | | | |
| OX = 10090 GN = — PE = 2 SV = 1 | | | | | |
| H-2 class I histocompatibility antigen, | HA18_MOUSE | 37 | kDa | 1.8505 | 0 |
| Q8 alpha chain OS = Mus musculus | | | | | |
| (Mouse) OX = 10090 GN = H2-Q8 PE = 3 | | | | | |
| SV = 1 | | | | | |
| Ig-like domain-containing protein | G3UXE9_MOUSE | 45 | kDa | 5.5514 | 0 |
| OS = Mus musculus (Mouse) OX = 10090 | | | | | |
| GN = Gm8909 PE = 1 SV = 1 | | | | | |
| Filamin-B OS = Mus musculus (Mouse) | FLNB_MOUSE | 278 | kDa | 1.8505 | 0 |
| OX = 10090 GN = Flnb PE = 1 SV = 3 | | | | | |
| Filamin-C OS = Mus musculus (Mouse) | FLNC_MOUSE | 291 | kDa | 2.7757 | 0 |
| OX = 10090 GN = Flnc PE = 1 SV = 3 | | | | | |
| sp\|A2ASS6\|TITIN_MOUSE-DECOY | sp\|A2ASS6\|TITIN_MOUSE- | ? | | 0.92523 | 0 |
| | DECOY | | | | |
| Phospholipid transfer protein OS = Mus | PLTP_MOUSE | 54 | kDa | 9.2523 | 0 |
| musculus (Mouse) OX = 10090 GN = Pltp | | | | | |
| PE = 1 SV = 1 | | | | | |
| sp\|Q6ZWQ0\|SYNE2_MOUSE-DECOY | sp\|Q6ZWQ0\|SYNE2_MOUSE- | ? | | 0.92523 | 0 |
| | DECOY | | | | |
| Desmoplakin OS = Mus musculus (Mouse) | DESP_MOUSE | 333 | kDa | 0.92523 | 0 |
| OX = 10090 GN = Dsp PE = 1 SV = 1 | | | | | |
| Pericentrin OS = Mus musculus (Mouse) | PCNT_MOUSE | 329 | kDa | 0.92523 | 0 |
| OX = 10090 GN = Pont PE = 1 SV = 2 | | | | | |
| sp\|Q80SU7\|GVIN1_MOUSE-DECOY | sp\|Q80SU7\|GVIN1_MOUSE- | ? | | 1.8505 | 0 |
| | DECOY | | | | |
| Complement component 6 OS = Mus | E9Q6D8_MOUSE | 105 | kDa | 5.5514 | 0 |
| musculus (Mouse) OX = 10090 GN = C6 | | | | | |
| PE = 1 SV = 2 | | | | | |
| Transmembrane protein KIAA1109 | K1109_MOUSE | 555 | kDa | 0.92523 | 0 |
| OS = Mus musculus (Mouse) OX = 10090 | | | | | |
| GN = Kiaa1109 PE = 1 SV = 4 | | | | | |
| sp\|Q3UHF7\|ZEP2_MOUSE-DECOY | sp\|Q3UHF7\|ZEP2_MOUSE- | ? | | 0.92523 | 0 |
| | DECOY | | | | |
| sp\|A2BH40\|ARI1A_MOUSE-DECOY | sp\|A2BH40\|ARI1A_MOUSE- | ? | | 0.92523 | 0 |
| | DECOY | | | | |
| E3 ubiquitin-protein ligase MIB1 | MIB1_MOUSE | 110 | kDa | 0.92523 | 0 |
| OS = Mus musculus (Mouse) OX = 10090 | | | | | |
| GN = Mib1 PE = 1 SV = 1 | | | | | |
| tr\|B1AR51\|B1AR51_MOUSE-DECOY | tr\|B1AR51\|B1AR51_MOUSE- | ? | | 0.92523 | 0 |
| | DECOY | | | | |
| Cluster of Tubulin beta-4B chain | TBB4B_MOUSE | 50 | kDa | 2.7757 | 0 |
| OS = Mus musculus (Mouse) OX = 10090 | [4] | | | | |
| GN = Tubb4b PE = 1 SV = 1 | | | | | |
| (TBB4B_MOUSE) | | | | | |
| Tubulin beta-4B chain OS = Mus | TBB4B_MOUSE | 50 | kDa | 2.7757 | 0 |
| musculus (Mouse) OX = 10090 | (+1) | | | | |
| GN = Tubb4b PE = 1 SV = 1 | | | | | |
| Tubulin beta-3 chain OS = Mus | TBB3_MOUSE | 50 | kDa | 1.8505 | 0 |
| musculus (Mouse) OX = 10090 GN = Tubb3 | | | | | |
| PE = 1 SV = 1 | | | | | |
| Tubulin beta-2A chain OS = Mus | TBB2A_MOUSE | 50 | kDa | 1.8505 | 0 |
| musculus (Mouse) OX = 10090 | | | | | |
| GN = Tubb2a PE = 1 SV = 1 | | | | | |
| sp\|Q8R3B7\|BRD8_MOUSE-DECOY | sp\|Q8R3B7\|BRD8_MOUSE- | ? | | 0.92523 | 0 |
| | DECOY | | | | |
| sp\|Q6A078\|CE290_MOUSE-DECOY | sp\|Q6A078\|CE290_MOUSE- | ? | | 0.92523 | 0 |
| | DECOY | | | | |
| sp\|Q66JQ7\|KNL1_MOUSE-DECOY | sp\|Q66JQ7\|KNL1_MOUSE- | ? | | 0.92523 | 0 |
| | DECOY | | | | |
| sp\|A2ARZ3\|FSIP2_MOUSE-DECOY | sp\|A2ARZ3\|FSIP2_MOUSE- | ? | | 0.92523 | 0 |
| | DECOY | | | | |
| Myosin light polypeptide 6 OS = Mus | MYL6_MOUSE | 17 | kDa | 2.7757 | 0 |
| musculus (Mouse) OX = 10090 GN = Myl6 | | | | | |
| PE = 1 SV = 3 | | | | | |
| Cathelicidin antimicrobial peptide | CAMP_MOUSE | 19 | kDa | 3.7009 | 0 |
| OS = Mus musculus (Mouse) OX = 10090 | | | | | |
| GN = Camp PE = 1 SV = 2 | | | | | |
| Tubulin beta-1 chain OS = Mus musculus | TBB1_MOUSE | 50 | kDa | 0.92523 | 0 |
| (Mouse) OX = 10090 GN = Tubb1 PE = 1 | | | | | |
| SV = 1 | | | | | |
| Keratin, type I cytoskeletal 13 OS = Mus | K1C13_MOUSE | 48 | kDa | 0.92523 | 0 |
| musculus (Mouse) OX = 10090 GN = Krt13 | | | | | |
| PE = 1 SV = 2 | | | | | |
| sp\|Q9R269\|PEPL_MOUSE-DECOY | sp\|Q9R269\|PEPL_MOUSE- | ? | | 1.8505 | 0 |
| | DECOY | | | | |
| PDZ domain-containing protein OS = Mus | E9Q616_MOUSE | 604 | kDa | 0.92523 | 0 |
| musculus (Mouse) OX = 10090 GN = Ahnak | | | | | |
| PE = 1 SV = 1 | | | | | |
| sp\|Q61285\|ABCD2_MOUSE-DECOY | sp\|Q61285\|ABCD2_MOUSE- | ? | | 1.8505 | 0 |
| | DECOY | | | | |
| RIKEN cDNA 4930407110 gene OS = Mus | D3Z5T8_MOUSE | 168 | kDa | 0.92523 | 0 |
| musculus (Mouse) OX = 10090 | | | | | |
| GN = 4930407I10Rik PE = 1 SV = 2 | | | | | |
| Cluster of sp\|A2A432\|CUL4B_MOUSE- | sp\|A2A432\|CUL4B_MOUSE- | ? | | 0.92523 | 0 |
| DECOY | DECOY [2] | | | | |
| sp\|A2A432\|CUL4B_MOUSE-DECOY | sp\|A2A432\|CUL4B_MOUSE- | ? | | 0.92523 | 0 |
| | DECOY | | | | |
| sp\|Q3TCH7\|CUL4A_MOUSE-DECOY | sp\|Q3TCH7\|CUL4A_MOUSE- | ? | | 0.92523 | 0 |
| | DECOY | | | | |
| Cystatin domain-containing protein | Q9D1B1_MOUSE | 17 | kDa | 3.7009 | 0 |
| OS = Mus musculus (Mouse) OX = 10090 | | | | | |
| GN = Cst6 PE = 1 SV = 1 | | | | | |
| VWFA domain-containing protein | E9Q7P1_MOUSE | 160 | kDa | 1.8505 | 0 |
| OS = Mus musculus (Mouse) OX = 10090 | | | | | |
| GN = Col22a1 PE = 1 SV = 1 | | | | | |
| sp\|Q60988\|STIL_MOUSE-DECOY | sp\|Q60988\|STIL_MOUSE- | ? | | 0.92523 | 0 |
| | DECOY | | | | |
| sp\|Q8WTY4\|CPIN1_MOUSE-DECOY | sp\|Q8WTY4\|CPIN1_MOUSE- | ? | | 0.92523 | 0 |
| | DECOY | | | | |
| Klotho OS = Mus musculus (Mouse) | KLOT_MOUSE | 116 | kDa | 0.92523 | 0 |
| OX = 10090 GN = Kl PE = 1 SV = 2 | | | | | |
| sp\|Q9R1L5\|MAST1_MOUSE-DECOY | sp\|Q9R1L5\|MAST1_MOUSE- | ? | | 1.8505 | 0 |
| | DECOY | | | | |
| Vasculin-like protein 1 OS = Mus | GPBL1_MOUSE | 52 | kDa | 0.92523 | 0 |
| musculus (Mouse) OX = 10090 | | | | | |
| GN = Gpbp1l1 PE = 1 SV = 1 | | | | | |
| Cluster of Ig kappa chain V-V region HP | KV5AB_MOUSE | 12 | kDa | 1.8505 | 0 |
| R16.7 OS = Mus musculus (Mouse) | [3] | | | | |
| OX = 10090 GN = — PE = 1 SV = 1 | | | | | |
| (KV5AB_MOUSE) | | | | | |
| Ig kappa chain V-V region HP R16.7 | KV5AB_MOUSE | 12 | kDa | 1.8505 | 0 |
| OS = Mus musculus (Mouse) OX = 10090 | (+1) | | | | |
| GN = — PE = 1 SV = 1 | | | | | |
| Ig kappa chain V-V region HP 123E6 | KV5AD_MOUSE | 12 | kDa | 0.92523 | 0 |
| OS = Mus musculus (Mouse) OX = 10090 | | | | | |
| GN = — PE = 1 SV = 1 | | | | | |
| Pyruvate kinase PKM OS = Mus musculus | KPYM_MOUSE | 58 | kDa | 2.7757 | 0 |
| (Mouse) OX = 10090 GN = Pkm PE = 1 | | | | | |
| SV = 4 | | | | | |
| Bridging integrator 2 OS = Mus musculus | BIN2_MOUSE | 53 | kDa | 1.8505 | 0 |
| (Mouse) OX = 10090 GN = Bin2 PE = 1 | | | | | |
| SV = 1 | | | | | |
| Angiotensinogen OS = Mus musculus | ANGT_MOUSE | 52 | kDa | 0.92523 | 0 |
| (Mouse) OX = 10090 GN = Agt PE = 1 SV = 1 | | | | | |
| Peptidase S1 domain-containing protein | Q792Z1_MOUSE | 26 | kDa | 0.92523 | 0 |
| OS = Mus musculus (Mouse) OX = 10090 | | | | | |
| GN = Try10 PE = 1 SV = 1 | | | | | |
| Apolipoprotein N OS = Mus musculus | G3X9D6_MOUSE | 28 | kDa | 0.92523 | 0 |
| (Mouse) OX = 10090 GN = Apon PE = 1 | | | | | |
| SV = 1 | | | | | |
| tr\|B2RPU8\|B2RPU8_MOUSE-DECOY | tr\|B2RPU8\|B2RPU8_MOUSE- | ? | | 0.92523 | 0 |
| | DECOY | | | | |
| Histone-lysine N-methyltransferase 2D | KMT2D_MOUSE | 600 | kDa | 0.92523 | 0 |
| OS = Mus musculus (Mouse) OX = 10090 | | | | | |
| GN = Kmt2d PE = 1 SV = 2 | | | | | |
| DNA topoisomerase 3-alpha OS = Mus | TOP3A_MOUSE | 112 | kDa | 0.92523 | 0 |
| musculus (Mouse) OX = 10090 GN = Top3a | | | | | |
| PE = 2 SV = 1 | | | | | |
| Cysteine and glycine-rich protein 1 | CSRP1_MOUSE | 21 | kDa | 1.8505 | 0 |
| OS = Mus musculus (Mouse) OX = 10090 | | | | | |
| GN = Csrp1 PE = 1 SV = 3 | | | | | |
| Vascular cell adhesion protein 1 OS = Mus | VCAM1_MOUSE | 81 | kDa | 0.92523 | 0 |
| musculus (Mouse) OX = 10090 | | | | | |
| GN = Vcam1 PE = 1 SV = 1 | | | | | |
| Apolipoprotein A-V OS = Mus musculus | APOA5_MOUSE | 41 | kDa | 0.92523 | 0 |
| (Mouse) OX = 10090 GN = Apoa5 PE = 1 | | | | | |
| SV = 1 | | | | | |
| Flavin reductase (NADPH) OS = Mus | BLVRB_MOUSE | 22 | kDa | 0.92523 | 0 |
| musculus (Mouse) OX = 10090 GN = Blvrb | | | | | |
| PE = 1 SV = 3 | | | | | |
| Dual specificity protein phosphatase 3 | DUS3_MOUSE | 20 | kDa | 0.92523 | 0 |
| OS = Mus musculus (Mouse) OX = 10090 | | | | | |
| GN = Dusp3 PE = 1 SV = 1 | | | | | |
| Peptidase S1 domain-containing protein | Q9CPN9_MOUSE | 26 | kDa | 0.92523 | 0 |
| OS = Mus musculus (Mouse) OX = 10090 | | | | | |
| GN = 2210010C04Rik PE = 1 SV = 1 | | | | | |
| Ras-related protein Rab-8B OS = Mus | RAB8B_MOUSE | 24 | kDa | 0.92523 | 0 |
| musculus (Mouse) OX = 10090 GN = Rab8b | | | | | |
| PE = 1 SV = 1 | | | | | |
| Nucleolar and coiled-body | NOLC1_MOUSE | 74 | kDa | 0.92523 | 0 |
| phosphoprotein 1 OS = Mus musculus | | | | | |
| (Mouse) OX = 10090 GN = Nolc1 PE = 1 | | | | | |
| SV = 1 | | | | | |
| sp\|B2RPV6\|MMRN1_MOUSE-DECOY | sp\|B2RPV6\|MMRN1_MOUSE- | ? | | 0.92523 | 0 |
| | DECOY | | | | |
| Proprotein convertase subtilisin/kexin | PCSK9_MOUSE | 75 | kDa | 0.92523 | 0 |
| type 9 OS = Mus musculus (Mouse) | | | | | |
| OX = 10090 GN = Pcsk9 PE = 1 SV = 2 | | | | | |
| sp\|P97350\|PKP1_MOUSE-DECOY | sp\|P97350\|PKP1_MOUSE- | ? | | 0.92523 | 0 |
| | DECOY | | | | |
| Isoleucine--tRNA ligase, mitochondrial | SYIM_MOUSE | 113 | kDa | 0.92523 | 0 |
| OS = Mus musculus (Mouse) OX = 10090 | | | | | |
| GN = Iars2 PE = 1 SV = 1 | | | | | |
| sp\|Q7TQC5\|APTX_MOUSE-DECOY | sp\|Q7TQC5\|APTX_MOUSE- | ? | | 0.92523 | 0 |
| | DECOY | | | | |
| sp\|O88329\|MYO1A_MOUSE-DECOY | sp\|O88329\|MYO1A_MOUSE- | ? | | 0.92523 | 0 |
| | DECOY | | | | |
| sp\|Q9D920\|BORC5_MOUSE-DECOY | sp\|Q9D920\|BORC5_MOUSE- | ? | | 0.92523 | 0 |
| | DECOY | | | | |
| Cilia- and flagella-associated protein 221 | PCDP1_MOUSE | 97 | kDa | 0.92523 | 0 |
| OS = Mus musculus (Mouse) OX = 10090 | | | | | |
| GN = Cfap221 PE = 1 SV = 1 | | | | | |
| Ig-like domain-containing protein | A0A075B5N9_MOUSE | 11 | kDa | 2.7757 | 0 |
| OS = Mus musculus (Mouse) OX = 10090 | | | | | |
| GN = Igkv3-7 PE = 4 SV = 7 | | | | | |
| Ig kappa chain V-III region PC | KV3A8_MOUSE | 12 | kDa | 2.7757 | 0 |
| 3741/TEPC 111 OS = Mus musculus | | | | | |
| (Mouse) OX = 10090 GN = — PE = 1 SV = 1 | | | | | |
| Ig-like domain-containing protein | A0A075B5K0_MOUSE | 13 | kDa | 0.92523 | 0 |
| OS = Mus musculus (Mouse) OX = 10090 | | | | | |
| GN = Igkv14-126 PE = 4 SV = 7 | | | | | |
| Ig-like domain-containing protein | A0A075B5K2_MOUSE | 10 | kDa | 0.92523 | 0 |
| OS = Mus musculus (Mouse) OX = 10090 | | | | | |
| GN = Igkv9-124 PE = 1 SV = 7 | | | | | |
| Ig-like domain-containing protein | A0A075B5R5_MOUSE | 13 | kDa | 0.92523 | 0 |
| OS = Mus musculus (Mouse) OX = 10090 | | | | | |
| GN = Ighv4-1 PE = 1 SV = 7 | | | | | |
| Ig-like domain-containing protein | A0A075B674_MOUSE | 11 | kDa | 0.92523 | 0 |
| OS = Mus musculus (Mouse) OX = 10090 | | | | | |
| GN = Ighv1-78 PE = 1 SV = 1 | | | | | |
| Ig-like domain-containing protein | A0A0A6YYE7_MOUSE | 13 | kDa | 0.92523 | 0 |
| OS = Mus musculus (Mouse) OX = 10090 | (+1) | | | | |
| GN = Igkv4-57 PE = 4 SV = 2 | | | | | |
| Angiopoietin-1 OS = Mus musculus | ANGP1_MOUSE | 58 | kDa | 0.92523 | 0 |
| (Mouse) OX = 10090 GN = Angpt1 PE = 1 | | | | | |
| SV = 2 | | | | | |
| Cofilin-1 OS = Mus musculus (Mouse) | COF1_MOUSE | 19 | kDa | 0.92523 | 0 |
| OX = 10090 GN = Cfl1 PE = 1 SV = 3 | | | | | |
| EH domain-containing protein 4 | EHD4_MOUSE | 61 | kDa | 0.92523 | 0 |
| OS = Mus musculus (Mouse) OX = 10090 | | | | | |
| GN = Ehd4 PE = 1 SV = 1 | | | | | |
| Ras-related C3 botulinum toxin substrate | RAC1_MOUSE | 21 | kDa | 0.92523 | 0 |
| 1 OS = Mus musculus (Mouse) OX = 10090 | (+2) | | | | |
| GN = Rac1 PE = 1 SV = 1 | | | | | |
| Basigin OS = Mus musculus (Mouse) | BASI_MOUSE | 42 | kDa | 0.92523 | 0 |
| OX = 10090 GN = Bsg PE = 1 SV = 2 | | | | | |
| SPARC-like protein 1 OS = Mus musculus | SPRL1_MOUSE | 72 | kDa | 0.92523 | 0 |
| (Mouse) OX = 10090 GN = Sparcl1 PE = 1 | | | | | |
| SV = 3 | | | | | |
| sp\|Q8K370\|ACD10_MOUSE-DECOY | sp\|Q8K370\|ACD10_MOUSE- | ? | | 0.92523 | 0 |
| | DECOY | | | | |
| sp\|Q99N80\|SYTL1_MOUSE-DECOY | sp\|Q99N80\|SYTL1_MOUSE- | ? | | 0.92523 | 0 |
| | DECOY | | | | |
| Mannan-binding lectin serine protease 1 | MASP1_MOUSE | 80 | kDa | 0.92523 | 0 |
| OS = Mus musculus (Mouse) OX = 10090 | | | | | |
| GN = Masp1 PE = 1 SV = 2 | | | | | |
| BPI fold-containing family A member 1 | BPIA1_MOUSE | 29 | kDa | 0.92523 | 0 |
| OS = Mus musculus (Mouse) OX = 10090 | | | | | |
| GN = Bpifa1 PE = 1 SV = 3 | | | | | |
| Cardiotrophin-like cytokine factor 1 | CLCF1_MOUSE | 25 | kDa | 0.92523 | 0 |
| OS = Mus musculus (Mouse) OX = 10090 | | | | | |
| GN = Clcf1 PE = 1 SV = 1 | | | | | |
| A disintegrin and metallopeptidase | Q6IMH7_MOUSE | 85 | kDa | 0.92523 | 0 |
| domain 6B OS = Mus musculus (Mouse) | | | | | |
| OX = 10090 GN = Adam6b PE = 4 SV = 1 | | | | | |
| Polypeptide N- | GLT13_MOUSE | 64 | kDa | 0.92523 | 0 |
| acetylgalactosaminyltransferase 13 | | | | | |
| OS = Mus musculus (Mouse) OX = 10090 | | | | | |
| GN = Galnt13 PE = 2 SV = 1 | | | | | |
| Coagulation factor XIII B chain OS = Mus | F13B_MOUSE | 76 | kDa | 0.92523 | 0 |
| musculus (Mouse) OX = 10090 GN = F13b | | | | | |
| PE = 1 SV = 2 | | | | | |
| Antileukoproteinase OS = Mus musculus | SLPI_MOUSE | 14 | kDa | 0.92523 | 0 |
| (Mouse) OX = 10090 GN = Slpi PE = 1 SV = 1 | | | | | |
| tr\|E9Q6R1\|E9Q6R1_MOUSE-DECOY | tr\|E9Q6R1\|E9Q6R1_MOUSE- | ? | | 0.92523 | 0 |
| | DECOY | | | | |
| Proteasome subunit alpha type-2 | PSA2_MOUSE | 26 | kDa | 0.92523 | 0 |
| OS = Mus musculus (Mouse) OX = 10090 | | | | | |
| GN = Psma2 PE = 1 SV = 3 | | | | | |
| sp\|Q8CFS6\|KCNV2_MOUSE-DECOY | sp\|Q8CFS6\|KCNV2_MOUSE- | ? | | 0.92523 | 0 |
| | DECOY | | | | |
| sp\|O08550\|KMT2B_MOUSE-DECOY | sp\|O08550\|KMT2B_MOUSE- | ? | | 0.92523 | 0 |
| | DECOY | | | | |
| Discoidin domain-containing receptor 2 | DDR2_MOUSE | 96 | kDa | 0.92523 | 0 |
| OS = Mus musculus (Mouse) OX = 10090 | | | | | |
| GN = Ddr2 PE = 1 SV = 2 | | | | | |

### Example 3. Conclusion

Pre-coated sera HBPE-NPs can be utilized for effective delivery to TNBC cells over non pre-coated PEG-HBPE-NPs. We've applied a novel HBPE polymer material for studying the effects of normal and IAV-infected mouse sera on NP cellular delivery. A corona's effects on potential endothelial migration, immune cell evasion, and in vivo distribution were also assessed. Enhancing coronal-mediated tumor delivery may translate to enhanced cancer drug delivery as well. When HBPE-NPs were pre-coated with normal or IAV sera, corona formation was confirmed by observing increases in NP diameter after anti-IgG antibody incubation. This verified IgG presence on NPs. Coronal protein patterns were visualized with gel electrophoresis. Distinct differences in protein abundance were seen among IAV sera (V3-V6). This indicates each viral serum created a unique biological identity on HBPE-NPs. Pre-coated normal and IAV sera HBPE-NPs displayed higher MDA-MB-231 uptake over PEG-HBPE-NPs. IAV sera (V4-V6) additionally reduced HBPE-NP delivery to THP-1 monocytes. This observance indicates certain IAV sera provide dysopsonin-like properties to HBPE-NPs. To simulate an in vivo environment, transwell cell culture systems were employed. These entailed a HUVEC-seeded upper chamber and MDA-MB-231-seeded bottom chamber. Normal or IAV-sera (V3-V6) exhibited higher localization in MDA-MB-231s over PEG-HBPE-NPs. HBPE-NPs (V5-V6) showed the greatest cancer cell uptake. This supports coronas can influence the transendothelial migration of NPs. Enhanced migration might correlate with higher endothelial trafficking in vivo. In mice, IAV (V5)-coated PEG-HBPE-NPs and IAV (V5)-coated HBPE-NPs demonstrated higher tumor delivery over non-pre-coated controls. HBPE-NPs (V5) had comparable tumor uptake to PEG-HBPE-NPs. HBPE-NPs (V5) further showed reduced spleen, kidney, and liver accumulation over PEG-HBPE-NPs. Elevated tumor transport might be attributed to IAV coronas enhancing NP circulation time, through improved immune evasion. Alternatively, IAV coronas may increase a NP's affinity to endothelial or tumor membranes. These results signify that non-PEG nanoconstructs can be employed for in vivo treatments. Moreover, coronas can affect HBPE-NP's biological behavior. Taken together, HBPE-NPs' (V5) increased delivery to cancer cells can be a result of their enrichment with cancer-honing proteins over PEG-HBPE-NPs.

In summary, pre-coating NPs with normal or IAV-infected sera can create a unique set of proteins that provide NPs with a distinct biological identity. This identity differs when NPs are first exposed to proteins during cell culture treatment or in vivo administration, such as PEG-HBPE-NPs. Different biological identities can impact HBPE-NP cellular behavior, including tumor trafficking. The majority of NP studies examine how intrinsic NP properties affect corona composition and cellular behavior using a healthy/normal protein source. Few studies have explored NP uptake in cells involving infection-related protein sources. For instance, complement C3b-coated NPs have exhibited enhanced lung delivery in pneumonic mice. These results showed that an inflammation-associated corona source can influence in vivo NP behavior. This is the first to study the impact of virus-infected sera on NP distribution to cells. This study is the first to explore the impact of innate (V3+V4) and adaptive (V5+V6) immune response-derived sera on NP cellular delivery. Furthermore, novel applications of viral sera coronas to transwell assays and mice delivery were performed. Using immune response-derived sera allows NPs to be enriched with proteins that cannot be as abundant in normal sera. Thus, new proteins with cancer-honing capabilities can be discovered through use of unique protein sources. Additionally, this study is the first to demonstrate that virus-infected sera can alter the cellular behavior of our exclusive polymer, HBPE. Using a unique NP material and a unique coronal protein source can lead to discovery of new potential targets for cancer cell receptors. This knowledge can lead to creation of innovative cancer therapeutics and treatment strategies.

These impacts on the nanotechnology field of enhancing cancer drug delivery efficiency. Cancer drugs can be chemical modified with peptides derived from HBPE's enriched proteins of interest that target cancer receptors. Cancer drugs can also be conjugated to these particular proteins. NPs can additionally be coated solely with groups of enriched tumor-targeting proteins. Designing NP materials with greater affinity for cancer-favoring proteins may be beneficial as well. Exploring NP coatings in other immune-related conditions, such as sepsis or COVID-19, may additionally provide unique protein combinations that aid cancer interaction. These research options can enhance tumor delivery and might consequently reduce non-specific uptake in healthy tissues.

In conclusion, IAV sera-derived coronas are a new and exciting means for improving a NP's cancer uptake and tumor trafficking. Designing optimal protein coronas enriched with cancer-honing properties can enhance drug delivery efficiency and in turn treatment outcomes.

1. A coronal protein-coated nanoparticle comprising one or more proteins, wherein the nanoparticle is a hyperbranched polyester (HBPE) nanoparticle, and wherein the one or more proteins are selected from the group consisting of Albumin, Complement C3, Pregnancy zone protein, Apolipoprotein A-I, Cluster of GLOBIN domain-containing protein, GLOBIN domain-containing protein, Serotransferrin, Ceruloplasmin, Cluster of Murinoglobulin-1, Murinoglobulin-1, Hemopexin, Cluster of Serine protease inhibitor A3K, Serine protease inhibitor A3K, Kininogen-1, Hemoglobin subunit beta-2, Plasminogen, Haptoglobin, Cluster of Alpha-1-antitrypsin 1-4, Alpha-2-HS-glycoprotein, Thrombospondin-1, Hemoglobin subunit alpha, Alpha-1-antitrypsin 1-4, Murinoglobulin-2, Alpha-1-antitrypsin 1-1, Alpha-1-antitrypsin 1-2, Inter alpha-trypsin inhibitor, heavy chain 4, Fibronectin, Vitamin D-binding protein, Prothrombin, Spectrin beta chain, erythrocytic, Serine protease inhibitor A3M, Cluster of Complement factor H, Complement factor H, Apolipoprotein E, Apolipoprotein A-IV, Cluster of Carboxylesterase 1C, Complement factor B, Beta-2-glycoprotein 1, Cluster of Ankyrin-1, Ankyrin-1, Spectrin alpha chain, erythrocytic 1, Carboxylesterase 1C, Apolipoprotein B-100, Serine protease inhibitor A3N, Inhibitor of carbonic anhydrase, Complement factor I, Complement C4-B, Histidine-rich glycoprotein, Cluster of Actin, cytoplasmic 1, Actin, cytoplasmic 1, Afamin, Inter-alpha-trypsin inhibitor heavy chain H2, Protein AMBP, Actin, alpha cardiac muscle 1, Inter-alpha-trypsin inhibitor heavy chain H3, LRRCT domain-containing protein, Vitronectin, Clusterin, Phosphatidylinositol-glycan-specific phospholipase D, Plasma kallikrein, Complement C5, Cluster of Keratin, type II cytoskeletal 2 epidermal, Cluster of H-2 class I histocompatibility antigen, Q10 alpha chain, H-2 class I histocompatibility antigen, Q10 alpha chain, Band 3 anion transport protein, Carboxylic ester hydrolase, Immunoglobulin heavy constant mu, Inter-alpha-trypsin inhibitor heavy chain H1, Serum amyloid P-component, Apolipoprotein M, Beta-actin-like protein 2, Gelsolin, Antithrombin-III, Alpha-1B-glycoprotein, CD5 antigen-like, Sulfhydryl oxidase 1, HMW kininogen-II, Ig-like domain-containing protein, Fetuin-B, Properdin, Ig gamma-2B chain C region, Complement component C8 alpha chain, Cluster of Heat shock cognate 71 kDa protein, Serum amyloid A-1 protein, Immunoglobulin kappa constant, Glutathione peroxidase 3, Heat shock cognate 71 kDa protein, Alpha-1-antitrypsin 1-5, Apolipoprotein A-II, Talin-1, Coagulation factor V, Carboxypeptidase N subunit 2, Fibrinogen alpha chain, Ig-like domain-containing protein, Coagulation factor X, Immunoglobulin heavy constant gamma 3, Carbonic anhydrase 1, Keratin, type II cytoskeletal 5, Apolipoprotein D, Ig gamma-1 chain C region, membrane-bound form, Apolipoprotein C-IV, Alpha-2-antiplasmin, Beta-2-microglobulin, Immunoglobulin heavy constant gamma 2C, Alpha-1-acid glycoprotein 1, Complement component C8 beta chain, N-acetylmuramoyl-L-alanine amidase, Vitamin K-dependent protein Z, Cluster of Filamin-A, Filamin-A, BPI fold-containing family A member 2, Serum paraoxonase/arylesterase 1, Serum amyloid A-4 protein, Epidermal growth factor receptor, Zinc-alpha-2-glycoprotein, Phosphatidylcholine-sterol acyltransferase, Mannose-binding protein C, Carboxypeptidase N catalytic chain, SCY domain-containing protein, Interleukin-1 receptor accessory protein, Immunoglobulin J chain, Complement C1q subcomponent subunit A, Keratin, type II cytoskeletal 2 epidermal, Keratin, type II cytoskeletal 1, IF rod domain-containing protein, Keratin, type II cytoskeletal 1b, Keratin, type II cytoskeletal 75, Carboxylic ester hydrolase, Fibrinogen gamma chain, Cluster of Keratin, type I cytoskeletal 10, Keratin, type I cytoskeletal 10, Serum amyloid A-2 protein, Transthyretin, Carbonic anhydrase 2, Immunoglobulin heavy constant alpha, Plasma protease C1 inhibitor, Transitional endoplasmic reticulum ATPase, Coagulation factor XII, Glyceraldehyde-3-phosphate dehydrogenase, Mannan-binding lectin serine protease 2, Alpha-1-acid glycoprotein 2, sp|Q8BXA7|PHLP2_DECOY, Keratin, type II cytoskeletal 73, IF rod domain-containing protein, Keratin, type II cytoskeletal 2 oral, Apolipoprotein C-III, Predicted gene 4788, Endoplasmic reticulum chaperone BiP, IF rod domain-containing protein, Selenoprotein P, Platelet-activating factor acetylhydrolase, Complement component C9, sp|P46656|ADX_DECOY, Corticosteroid-binding globulin, Platelet factor 4, Cluster of Alpha-amylase 1, Alpha-amylase 1, Heparin cofactor 2, Ig-like domain-containing protein, Cluster of Tubulin alpha-4A chain, Tubulin alpha-4A chain, Complement factor D, Retinol-binding protein 4, 14-3-3 protein zeta/delta, Secreted phosphoprotein 24, Macrophage colony-stimulating factor 1 receptor, Lumican, 55 kDa erythrocyte membrane protein, sp|Q9WTU0|PHF2_DECOY, sp|P59242|CING_DECOY, C-reactive protein, Coagulation factor IX, Peroxiredoxin-2, Vitamin K-dependent protein S, sp|Q571H0|NPA1P_DECOY, Ficolin-1, Ig lambda-2 chain C region, Profilin-1, Extracellular superoxide dismutase [Cu—Zn], Complement C1q subcomponent subunit C, Biotinidase, Keratin, type II cytoskeletal 79, Keratin, type II cytoskeletal 8, Keratin, type II cytoskeletal 74, Ankyrin-3, Ankyrin-2, C4b-binding protein, Apolipoprotein C-I, Keratin, type I cytoskeletal 16, Pigment epithelium-derived factor, Endogenous retroviral sequence 3, Predicted gene 382, Cluster of Myosin-9, Myosin-9, Myosin-11, sp|P98083|SHC1_DECOY, Hepatocyte growth factor activator, Pancreatic alpha-amylase, Aamy domain-containing protein, Ig-like domain-containing protein, Keratin, type I cytoskeletal 28, Spectrin alpha chain, non-erythrocytic 1, Extracellular matrix protein 1, Fibrinogen beta chain, Mannose-binding protein A, Carboxypeptidase B2, Tubulin alpha-1B chain, Complement component C8 gamma chain, Keratin, type I cytoskeletal 42, Protein PRRC2B, Lysosomal alpha-mann, OSidase, sp|Q6RUT8|CC154_DECOY, sp|Q8BMD6|TM260_DECOY, Ras-related protein Rap-1b, Alpha-actinin-1, Proteoglycan 4, Ig-like domain-containing protein, von Willebrand factor, Serglycin, sp|A2AAY5|SPD2B_DECOY, sp|Q9JLV2|TP4AP_DECOY, Hyaluronan-binding protein 2, Disks large homolog 5, sp|Q7TSJ2|MAP6_DECOY, Amyloid-beta A4 precursor protein-binding family B member 2, sp|Q9Z1P8|ANGL4_DECOY, tr|J3QNP2|J3QNP2_DECOY, Insulin-like growth factor-binding protein complex acid labile subunit, Fermitin family homolog 3, Ig-like domain-containing protein, Ig kappa chain V-II region 26-10, Ig kappa chain V-V region MOPC 41, L-selectin, Ig kappa chain V-VI region XRPC 44, Leukemia inhibitory factor receptor, Platelet glycoprotein Ib alpha chain, Hepatocyte growth factor-like protein, Nuclear receptor subfamily 1 group D member 2, sp|B2RQE8|RHG42_DECOY, Lysozyme C-2, Tubulin alpha-8 chain, Vitamin K-dependent protein C, RIKEN cDNA 9530053A07 gene, Ig-like domain-containing protein, Ig-like domain-containing protein, Superoxide dismutase [Cu—Zn], Triosephosphate isomerase, Anaphylatoxin-like domain-containing protein, Hepcidin, Peptidyl-prolyl cis-trans isomerase A, Metalloproteinase inhibitor 3, Ig-like domain-containing protein, Immunoglobulin heavy variable 1-15, Progranulin, Testisin, tr|Q91X36|Q91X36_DECOY, sp|Q8VEE4|RFA1_DECOY, Complement C1r-A subcomponent, Carboxypeptidase Q, Dematin, Ig heavy chain V region MOPC 47A, T-lymphoma invasion and metastasis-inducing protein 1, Ras-related protein Rab-19, Protein disulfide-isomerase A3, Proto-oncogene tyrosine-protein kinase receptor Ret, Ig-like domain-containing protein, Monocarboxylate transporter 1, sp|P67984|RL22_DECOY, Phosphatidylinositide phosphatase SAC2, Complement factor H-related 2, Serine protease inhibitor A3G, Neurofilament heavy polypeptide, H-2 class I histocompatibility antigen, alpha chain, H-2 class I histocompatibility antigen, Q8 alpha chain, Ig-like domain-containing protein, Filamin-B, Filamin-C, sp|A2ASS6|TITIN_DECOY, phospholipid transfer protein, sp|Q6ZWQ0|SYNE2_DECOY, Desmoplakin, Pericentrin, sp|Q80SU7|GVIN1_DECOY, Complement component 6, Transmembrane protein KIAA1109, sp|Q3UHF7|ZEP2_DECOY, sp|A2BH40|ARI1A_DECOY, E3 ubiquitin-protein ligase MIB1, tr|B1AR51|B1AR51_DECOY, Cluster of Tubulin beta-4B chain, Tubulin beta-4B chain, Tubulin beta-3 chain, Tubulin beta-2A chain, sp|Q8R3B7|BRD8_DECOY, sp|Q6A078|CE290_DECOY, sp|Q66JQ7|KNL1_DECOY, sp|A2ARZ3|FSIP2_DECOY, Myosin light polypeptide 6, Cathelicidin antimicrobial peptide, Tubulin beta-1 chain, Keratin, type I cytoskeletal 13, sp|Q9R269|PEPL_DECOY, PDZ domain-containing protein, sp|Q61285|ABCD2_DECOY, RIKEN cDNA 4930407110 gene, Cluster of sp|A2A432|CUL4B_DECOY, sp|A2A432|CUL4B_DECOY, sp|Q3TCH7|CUL4A_DECOY, Cystatin domain-containing protein, VWFA domain-containing protein, sp|Q60988|STIL_DECOY, sp|Q8WTY4|CPIN1_DECOY, Klotho, sp|Q9R1L5|MAST1_DECOY, Vasculin-like protein 1, Cluster of Ig kappa chain V-V region HP R16.7, Ig kappa chain V-V region HP R16.7, Ig kappa chain V-V region HP 123E6, Pyruvate kinase PKM, Bridging integrator 2, Angiotensinogen, Peptidase Si domain-containing protein, Apolipoprotein N, tr|B2RPU8|B2RPU8_DECOY, Histone-lysine N-methyltransferase 2D, DNA topoisomerase 3-alpha, Cysteine and glycine-rich protein 1, Vascular cell adhesion protein 1, Apolipoprotein A-V, Flavin reductase (NADPH), Dual specificity protein phosphatase 3, Peptidase Si domain-containing protein, Ras-related protein Rab-8B, Nucleolar and coiled-body phosphoprotein 1, sp|B2RPV6|MMRN1_DECOY, Proprotein convertase subtilisin/kexin type 9, sp|P97350|PKP1_DECOY, Isoleucine-tRNA ligase, mitochondrial, sp|Q7TQC5|APTX_DECOY, sp|O88329|MYO1A_DECOY, sp|Q9D920|BORC5_DECOY, Cilia- and flagella-associated protein 221, Ig-like domain-containing protein, Ig kappa chain V-III region PC 3741/TEPC 111, Ig-like domain-containing protein, Ig-like domain-containing protein, Ig-like domain-containing protein, Ig-like domain-containing protein, Ig-like domain-containing protein, Angiopoietin-1, Cofilin-1, EH domain-containing protein 4, Ras-related C3 botulinum toxin substrate 1, Basigin, SPARC-like protein 1, sp|Q8K370|ACD10_DECOY, sp|Q99N80|SYTL1_DECOY, Mannan-binding lectin serine protease 1, BPI fold-containing family A member 1, Cardiotrophin-like cytokine factor 1, A disintegrin and metallopeptidase domain 6B, Polypeptide N-acetylgalactosaminyltransferase 13, Coagulation factor XIII B chain, Antileukoproteinase, tr|E9Q6R1|E9Q6R1_DECOY, Proteasome subunit alpha type-2, sp|Q8CFS6|KCNV2_DECOY, sp|O08550|KMT2B_DECOY, and Discoidin domain-containing receptor 2, a fragment thereof or a variant thereof.

2. The coronal protein-coated nanoparticle of claim 1, wherein the one or more proteins are selected from the group consisting of complement C3, alpha-2-HS-glycoprotein, complement factor B, vitronectin, clusterin, inhibitor of carbonic anhydrase, H-2 class I histocompatibility antigen, Q10 alpha chain, complement C5, carboxypeptidase N subunit 2, plasma protease C1 inhibitor, alpha-1-acid glycoprotein 1, alpha-2-antiplasmin, complement component C8 alpha chain, complement component C9, serum amyloid A-1 protein, complement factor D, serum amyloid A-2 protein, Ig-like domain-containing protein, complement C1s-A subcomponent, N-acetylmuramoyl-L-alanine amidase, carboxypeptidase N catalytic chain, complement C2, complement component 7, mannan-binding lectin serine protease 2, ficolin-1, complement C1r-A subcomponent, vitamin K-dependent protein S, mannan-binding lectin serine protease 1, glyceraldehyde-3-phosphate dehydrogenase, vitamin K-dependent protein C, interleukin-1 receptor accessory protein, fibronectin, apolipoprotein B-100, complement factor H, haptoglobin, immunoglobulin heavy constant mu, complement component C8 beta chain, Ig gamma-2B chain C region, protein AMBP, Ig gamma-1 chain C region, membrane-bound form, complement component C8 gamma chain, alpha-1-acid glycoprotein 2, immunoglobulin kappa constant, mannose-binding protein C, beta-2-microglobulin, serum amyloid P-component, complement C1s-B subcomponent, transthyretin, inter alpha-trypsin inhibitor, heavy chain 4, Inter-alpha-trypsin inhibitor heavy chain H2, histidine-rich glycoprotein, afamin, apolipoprotein A-II, corticosteroid-binding globulin, flavin reductase (NADPH), pregnancy zone protein, beta-2-glycoprotein 1, ceruloplasmin, serum paraoxonase/arylesterase 1, glutathione peroxidase 3, insulin-like growth factor-binding protein complex acid labile subunit, apolipoprotein C-III, albumin, apolipoprotein A-I, apolipoprotein A-IV, apolipoprotein E, complement factor I, hemopexin, plasminogen, and thrombospondin-1, or a fragment thereof.

3. The coronal protein-coated nanoparticle of claim 1, wherein the one or more proteins are selected from the group consisting of Inter alpha-trypsin inhibitor, heavy chain 4, alpha-2-HS-glycoprotein, inter-alpha-trypsin inhibitor heavy chain H2, clusterin, histidine-rich glycoprotein, afamin, carboxypeptidase N subunit 2, apolipoprotein A-II, corticosteroid-binding globulin, and flavin reductase (NADPH), or a fragment thereof.

4. The coronal protein-coated nanoparticle of claim 1, wherein the one or more proteins are selected from the group consisting of pregnancy zone protein, apolipoprotein B-100, beta-2-glycoprotein 1, ceruloplasmin, serum paraoxonase/arylesterase 1, glutathione peroxidase 3, insulin-like growth factor-binding protein complex acid labile subunit, apolipoprotein C-III, beta-2-microglobulin, and mannose-binding protein C, or a fragment thereof.

5. The coronal protein-coated nanoparticle of claim 1, wherein the one or more proteins are selected from the group consisting of albumin, alpha-2-HS-glycoprotein, apolipoprotein A-I, apolipoprotein A-IV, apolipoprotein B-100, apolipoprotein E, beta-2-glycoprotein 1, ceruloplasmin, clusterin, complement factor I, hemopexin, histidine-rich glycoprotein, inter alpha-trypsin inhibitor, heavy chain 4, plasminogen, pregnancy zone protein, and thrombospondin-1, or a fragment thereof.

6. (canceled)

7. The coronal protein-coated nanoparticle of claim 1, wherein the one or more proteins are directly attached to the HBPE nanoparticle or are attached to the HBPE nanoparticle through a sequence that is attached directly to the HBPE nanoparticle.

8. (canceled)

9. The coronal protein-coated nanoparticle of claim 1, wherein the one or more proteins form a homogenous shell around the HBPE nanoparticle.

10. The coronal protein-coated nanoparticle of claim 1, w herein the coronal protein-coated nanoparticle is from about 100 nm to about 200 nm in size.

11. The coronal protein-coated nanoparticle of claim 1, further comprising an anti-cancer agent or an imaging compound.

12. The coronal protein-coated nanoparticle of claim 11, wherein the anti-cancer agent comprises taxol, paclitaxel, docetaxel, or cabazitaxel.

13. The coronal protein-coated nanoparticle of claim 11, wherein the anti-cancer agent is hydrophobic.

14. The coronal protein-coated nanoparticle of claim 1, wherein the anti-cancer agent is encapsulated in the interior of the nanoparticle.

15. The coronal protein-coated nanoparticle of claim 1, wherein the nanoparticle further comprises a chelating ligand.

16. The coronal protein-coated nanoparticle of claim 15, wherein the chelating ligand is desferrioxamine (DFO).

17. (canceled)

18. A method of treating cancer in a subject in need thereof, comprising administering to the subject a therapeutically effective amount of the coronal protein-coated nanoparticle of claim 1.

19. The method of claim 18, wherein the cancer is a breast cancer.

20. The method of claim 19, wherein the breast cancer is triple negative breast cancer.

21. A method of generating one or more coronal protein-coated nanoparticles, comprising contacting one or more nanoparticles with a serum sample obtained from a subject, wherein the serum samples is obtained from a subject infected by influenza A virus, wherein the nanoparticle is a hyperbranched polyester (HBPE) nanoparticle.

22. The method of claim 21, wherein the serum sample is obtained from the subject on day 3, day 4, day 5, day 6, day 7, or day 8 post infection of influenza A virus.

23. (canceled)

24. (canceled)

25. (canceled)

26. (canceled)

27. The method of claim 21, wherein the nanoparticles are in contact with the serum sample at a ratio of 20:1 (nanoparticles:sera) by volume.
